(19) United States
(12) Patent Application Publication
(10) Pub. No.: US 2022/0338454 A1
(43) Pub. Date: Oct. 27, 2022

(54) SYSTEMS AND METHODS FOR GENERATING GENETIC INCOMPATIBILITY

(71) Applicant: REGENTS OF THE UNIVERSITY OF MINNESOTA, Minneapolis, MN (US)

(72) Inventors: Michael Joseph SMANSKI, Falcon Heights, MN (US); Siba DAS, Falcon Heights, MN (US); Maciej MASELKO, Sydney (AU); Nathan Robert FELTMAN, Lauderdale, MN (US); Ambuj UPADHYAY, Falcon Heights, MN (US)

(21) Appl. No.: 17/763,870

(22) PCT Filed: Oct. 30, 2020
(86) PCT No.: PCT/US2020/058301

§ 371 Date: Mar. 25, 2022

Related U.S. Application Data
(60) Provisional application No. 62/928,612, filed on Oct. 31, 2019.

(51) Int. Cl. A01K 67/033; C12N 9/22; C12N 15/11; C12N 15/85; C12N 15/113

(52) U.S. Cl. CPC A01K 67/0339; C12N 9/22; C12N 15/11; C12N 15/8509; C12N 15/113; C12N 2830/001; C12N 2310/20; C12N 2800/80; C12N 2800/105; A01K 2227/706; A01K 2217/05

(57) ABSTRACT

An engineered genetic incompatibility (EGI) strain of a wild-type organism is designed to include a haplosufficient lethal allele and a haploinsufficient resistance allele. In another aspect, a biocontainment system generally includes a polynucleotide that encodes a coding region whose expression causes infertility or death, a transcription regulatory region operably linked upstream of the coding region and containing a silent mutation, and a polynucleotide that encodes a programmable transcription activator. The programmable transcription activator is engineered to bind to the transcription regulatory region in the absence of the silent mutation, thereby expressing the coding region in the absence of the silent mutation, but does not initiate expression of the coding region when the transcription regulatory region comprises the silent mutation.

### CROSS-REFERENCE TO RELATED APPLICATION

This application claims the benefit of U.S. Provisional Patent Application No. 62/928,612, filed Oct. 31, 2019, which is incorporated herein by reference in its entirety.

### GOVERNMENT FUNDING

This invention was made with government support under Grant No. HR0011836772 awarded by the Department of Defense/Defense Advanced Research Projects Agency (DARPA). The government has certain rights in the invention.

### SEQUENCE LISTING

This application contains a Sequence Listing electronically submitted to the United States Patent and Trademark Office via EFS-Web as an ASCII text file entitled “Seq_List-0110-000634WO01_ST25.txt” having a size of 67 kilobytes and created on Oct. 29, 2020. Due to the electronic filing of the Sequence Listing, the electronically submitted Sequence Listing serves as both the paper copy required by 37 CFR § 1.821(c) and the CRF required by § 1.821(e). The information contained in the Sequence Listing is incorporated by reference herein.

### SUMMARY

This disclosure describes, in one aspect, a biocontainment system. Generally, the biocontainment system includes a polynucleotide that encodes a coding region whose expression causes infertility or death, a transcription regulatory region operably linked upstream of the coding region and containing a silent mutation, and a polynucleotide that encodes a programmable transcription activator. The programmable transcription activator is engineered to bind to the transcription regulatory region in the absence of the silent mutation, thereby expressing the coding region in the absence of the silent mutation, but does not initiate expression of the coding region when the transcription regulatory region comprises the silent mutation.

In some embodiments, the programmable transcription activator includes dCas9 fused to an activation domain.

In some embodiments, the coding region encodes a cytoskeletal polypeptide, an ER-Golgi vesicle polypeptide, an mRNA processing polypeptide, an electron transport polypeptide, a nuclear trafficking polypeptide, a chromosome segregation polypeptide, a spindle pole duplication polypeptide, an oxidative stress polypeptide, or a polypeptide controlling development.

In another aspect, this disclosure describes a multicellular organism having germ cells homozygous for any embodiment of the biocontainment system summarized above.

In another aspect, this disclosure describes a method of limiting hybridization of a genetically-modified organism with a genetically dissimilar variant. Generally, the method includes providing an organism genetically modified to include any embodiment of the biocontainment system summarized above. A cross between the genetically-modified organism and the genetically dissimilar variant organism results in progeny that exhibit a phenotype that is distinct from the genetically-modified organism.

In some embodiments, the genetically dissimilar variant can be a wild-type organism.

In some embodiments, the genetically dissimilar variant can be engineered to have a different genetic modification compared to the genetically-modified organism having the biocontainment system.

In some embodiments, the phenotype exhibited by the progeny can be lethality or infertility.

In another aspect, this disclosure describes an engineered genetic incompatibility (EGI) strain of a multicellular organism. Generally, the EGI strain possesses a haplosufficient lethal allele and a haploinsufficient resistance allele. The haplosufficient lethal allele and a haploinsufficient resistance allele can be components of the biocontainment system summarized above.

In another aspect, this disclosure describes a method of suppressing a population of a wild-type organisms. Generally, the method includes providing an engineered genetic incompatibility (EGI) strain of the wild-type organism and mating members of the EGI strain of one sex with fertile adults of the opposite sex in the population of wild-type organisms. The EGI strain is engineered to include a haplosufficient lethal allele and a haploinsufficient resistance allele so that wild-type×EGI crosses produce at least 50% lethality. In some embodiments, the method can include additional matings between members of the EGI strain of the one sex with fertile adults of the opposite sex in the wild-type population.

In another aspect, this disclosure describes a method of replacing a population of wild-type organisms. Generally, the method includes providing an engineered genetic incompatibility (EGI) strain of the wild-type organism and mating the EGI strain with fertile adults in the population of wild-type organisms. The EGI strain is engineered to include a haplosufficient lethal allele and a haploinsufficient resistance allele so that wild-type×EGI crosses produce at least 50% lethality and EGI×EGI crosses produce at least 75% viability.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Design of Engineered Genetic Incompatibility (EGI). (A) Schematic diagram of genotypes used to generate for EGI. L, dominant lethal gene; 1, wild-type allele (null); S, dominant susceptible allele; s, recessive resistant allele. (B) X-ray crystal structure of S. pyogenes Cas9 (PDB ID: 6o0z, left) and diagram of dominant lethal gene product, dCas9-VPR. (C) Interaction of dCas9-VPR with resistant (top) or susceptible (bottom) alleles. Blue square represents a mutation that abrogates dCas9 binding. RNAP, RNA polymerase.

FIG. 2. Empirical determination of targets for lethal overexpression or ectopic expression. Results showing the number of progeny surviving to pupal stage (dark circles) or adult life-stage (light circles) for crosses between a paternal fly homozygous for a dCas9-VPR expression cassette (rows) and a maternal fly homozygous for sgRNA expression cassette (columns). Individual experiments are shaded according to phenotype categories according to the key below. n=2 biologically independent replicates.

FIG. 3. Genotype and hybrid incompatibility of select EGI strains. (A) Proximity of sgRNA binding sites to transcription start site (TSS) for EGI strains. Sequences of both sgRNA binding sites are shown below promoter illustration, with protospacers in red and protospacer adjacent motifs in blue. Sequences of the mutated promoters at the sgRNA binding loci are shown below with differences highlighted in grey shadow. (B) Chromosomal locations of genome alterations. Target genes, PTA-constructs, sgRNA constructs, and joint PTA-sgRNA constructs are labeled.

FIG. 4 Genotype and hybrid incompatibility of select EGI strains. (A) Hybrid incompatibility data showing number of progeny surviving to adulthood. Genotype of parental strains for each cross are given on the x-axis. n=3 biologically independent experiments. (B) Immunohistochemical staining of wild-type (left) or hybrid (right) larva showing overexpression or ectopic-expression of targeted signaling pathways. Antibody binding targets are labelled in the bottom left corner of each image. For each panel, the wg-targeting, pyr-targeting, and hh-targeting EGI genotypes are shown from top to bottom. 200 μm scale bar. Images are representative of at least six independent biological samples for each strain.

FIG. 5. Engineering multiple orthogonal EGI strains. Mating compatibility between wild-type and 12 EGI genotypes, reported as the number of adult offspring 15 days after mating. Female (maternal) genotype is listed on the left axis with the naming convention [target.PTApromoter.construction-method], and male (paternal) genotypes are presented in the same order along the top axis. Predicted compatible strains are indicated with black-outline boxes across the diagonal. Grey boxes indicate crosses that were not measured for lack of virgin females for hh.Pfoxo.injection and pyr.Ptub.injection strains. Superscript B denotes that the strain was later found to have floating Balancer chromosomes. Smaller grid at right highlights four mutually-compatible strains.

FIG. 6. Pwg promoter mutations. Pwg promoter mutant sequencing trace and alignment to wild-type promoter. Targeted protospacers indicated in red; Protospacer adjacent motifs (PAMs) indicated in blue.

FIG. 7. Ppyr promoter mutations. Ppyr promoter mutant sequencing trace and alignment to wild-type promoter. Targeted protospacers indicated in red; PAMs indicated in blue.

FIG. 8. Phh promoter mutations. Phh promoter mutant sequencing trace and alignment to wild-type promoter. Targeted protospacers indicated in red; PAMs indicated in blue.

FIG. 9. Crossing strategy to produce hh-EGI flies. (A) Genotypes and sex of flies involved in crosses to assemble EGI components. Crosses are indexed with numbered white circles. ‘X’ designates a recombination event required in the female parent of cross #9. The female from cross #18 resulted from cross #7. Embryos from cross #13 were injected with promoter::dCas9::VPR constructs and ΦC31 integrase. Question mark denotes a chromosome genotype that was not verified. (B) Illustrated and color-coded genotypes of key intermediates. BDSC #54591, BDSC #67560, and BDSC #9744 were purchased from the Bloomington Drosophila Stock Center. Star ST, SGSB, and C26b are balancer strains.

FIG. 10. Crossing strategy to produce wg-EGI flies. (A) Genotypes and sex of flies involved in crosses used to assemble EGI components. Crosses are indexed with numbered white circles. ‘X’ designates a recombination event required in the female parent of cross #7. Embryos from cross #1 were injected with a sgRNA-wg construct and ΦC31 integrase. Question mark denotes a chromosome genotype that was not verified. The males in cross #7, cross #8, and cross #11 and the female in cross #4 are offspring from FIG. 13, cross #4. The female in cross #11 is offspring from FIG. 9, cross #16. (B) Illustrated and color-coded genotypes of key intermediates. BDSC #9748 was purchased from the Bloomington Drosophila Stock Center. Star ST is a balancer strain.

FIG. 11. Reinjection strategy to produce hh-EGI flies. (A) Genotypes and sex of flies involved in crosses used to assemble EGI components. Crosses are indexed with numbered white circles. Embryos from cross #8 were injected with promoter::dCas9::VPR+sgRNA-hh constructs and ΦC31 integrase. Question mark denotes a chromosome genotype that was not verified. (B) Illustrated and color-coded genotypes of key intermediates. BDSC #54591, BDSC #67560, and BDSC #9752 were purchased from the Bloomington Drosophila Stock Center. Star ST, SGSB, and C26b are balancer strains.

FIG. 12. Reinjection strategy to produce pyr-EGI flies. (A) Genotypes and sex of flies involved in crosses used to assemble EGI components. Crosses are indexed with numbered white circles. Embryos from cross #8 were injected with promoter::dCas9::VPR+sgRNA-pyr constructs and ΦC31 integrase. Question mark denotes a chromosome genotype that was not verified. (B) Illustrated and color-coded genotypes of key intermediates. BDSC #54591, BDSC #67537, and BDSC #9748 were purchased from the Bloomington Drosophila Stock Center. Star ST, SGSB, and C26b are balancer strains.

FIG. 13. Reinjection strategy to produce wg-EGI flies. (A) Genotypes and sex of flies involved in crosses used to assemble EGI components. Crosses are indexed with numbered white circles. Embryos from cross #1 were injected with the sgRNA-wg expression construct. Embryos from cross #7 were injected with promoter::dCas9::VPR+sgRNA-wg constructs and ΦC31 integrase. Question mark denotes a chromosome genotype that was not verified. The male in cross #5 is offspring from FIG. 12, cross #7. (B) Illustrated and color-coded genotypes of key intermediates. BDSC #51324 was purchased from the Bloomington Drosophila Stock Center. yGlac and SGSB are balancer strains.

FIG. 14. Chromosomal maps of all EGI strains reported in this work.

FIG. 15. Characterization of EGI strains. (A) Chromosomal locations of genome alterations for EGI strains whose hybrid offspring were analyzed by immunohistochemistry. EGI strains illustrated here correspond to the ones used in FIG. 3 and FIG. 4. (B) Immunofluorescence staining of third instar larval brains from wild-type (left) or hybrid (right) showing overexpression or ectopic expression of targeted signaling pathways. Grayscale images show antibody staining for proteins encoded by lethal overexpression target (wingless, top) or downstream signaling pathway components (p-ERK1/2, middle and patched, bottom). Corresponding brightfield images of the brains to the right. Scale bar=200 μm.

FIG. 16. Release scheme for negatively correlating cross-resistance. Purple denotes wild-type pests, green and yellow denote mutually-incompatible EGI strains, for which only males would be released. Orange denotes resistant ‘escapees’, which inherit half of their genome from the previously released biocontrol EGI strain.

FIG. 17. Average number of offspring for intraspecific matings of each wildtype and EGI fly line.

FIG. 18. Mating phenotypes, as in FIG. 5, of two strains from FIG. 5 that were found to contain balancer chromosomes in the population but were later purified to true-breeding genotypes.

FIG. 19. Threshold dependent gene drive results. EGI and wild-type flies were co-housed in a single enclosure and carried forward generationally. At each generation, the frequency of EGI flies in the total population was quantified. Traces above are marked by starting population composition (20% EGI, unmarked line; 30%, open triangles; 40% closed squares; 50% open pentagons; 60%, closed hexagons; 70% open heptagons; 80% closed octagons). The threshold for population replacement based on this data is ˜75%.

FIG. 20. Genotype and performance of Self-sorting Incompatible Male System (SSIMS). (A) Genetic markers of Female Lethality (GFP) and EGI (red eyes) are combined in SSIMS line. (B) Percentage of male and female offspring in crosses from parents labeled on the x-axis, with or without tetracycline (Tet).

FIG. 21. Mating results from crosses with different numbers of wild-type males, SSIMS males, and five wild-type females. For each mating left bar is number of adults, middle bar is number of pupae, and right bar is number of eggs. Egg counts are the average per female, whereas the other numbers are combined.

FIG. 22. Mating competition results for EGI and wild-type males. Bars show average number of surviving adults progeny. Error bars show one standard deviation from at least three independent replicates.

### DETAILED DESCRIPTION OF ILLUSTRATIVE EMBODIMENTS

Speciation constrains the flow of genetic information between populations of sexually reproducing organisms. Gaining control over mechanisms of speciation enables new strategies to manage wild populations of biological organisms including, but not limited to, disease vectors, agricultural pests, and/or invasive species. Additionally, control over mechanisms of speciation can provide safe biocontainment of transgenes and gene drives.

Speciation in nature can be driven by pre-zygotic barriers that prevent maternal and paternal gametes from meeting or by post-zygotic incompatibilities that render the hybrid progeny inviable or sterile. This disclosure describes a general approach to create engineered genetic incompatibilities (EGIs) that direct speciation. In its most basic form, the system described herein couples a dominant lethal transgene with a recessive resistance allele. EGI strains that are homozygous for both elements are fertile and fecund when they mate with similarly engineered strains, but completely incompatible with wild-type.

This disclosure also shows that EGI genotypes can be tuned to cause hybrid lethality at different developmental life-stages. Further, this disclosure demonstrates that multiple orthogonal EGI strains of the model organism D. melanogaster can be engineered to be mutually incompatible with wild-type and with each other. The approach to create EGI organisms described herein is simple, robust, and functional in multiple sexually reproducing organisms.

In genetics, underdominance occurs when a heterozygous genotype (Aa) is less fit than either homozygous genotype (AA and aa) from which it was produced. Engineered underdominance can be leveraged for the control of wild populations such as, for example, the local suppression or replacement of a target population of disease vectors, agricultural pests, or invasive species. Several strategies for engineering underdominance are known, including one-locus or two-locus toxin-antitoxin systems, chromosomal translocations, and using RNAi to cause negative genetic interactions.

In ‘extreme underdominance,’ the heterozygote is inviable while each homozygote has equal fitness. Extreme underdominance can be leveraged as threshold-dependent, spatially contained gene drive. Relatively modest release rates—e.g., below 5% of the total per generation—can be sufficient to replace Aedes aegypti populations. Such a gene drive may be more socially acceptable than threshold-independent gene drives to suppress vector competence since they do not have the potential for uncontrolled spread. Alternatively, only males could be released for a genetic biocontrol approach that mimics sterile insect technique. Despite its theoretical utility in population control, extreme underdominance has been difficult to engineer.

Extreme underdominance amounts to a speciation event, as it prevents successful reproduction and therefore genetic exchange between the two homozygous populations. In nature, speciation events are driven by prezygotic and postzygotic incompatibilities. Prezygotic incompatibilities prevent fertilization from taking place. These can include geographic separation or behavioral/anatomical differences between individuals in two populations that prevent sperm and egg from meeting. Postzygotic incompatibilities occur when genetic or cellular differences between the maternal and paternal gametes render the fertilized egg inviable or infertile. The Dobzhansky-Muller Incompatibility (DMI) model asserts that postzygotic incompatibilities can arise via mutations that create a two-locus underdominance effect. DMIs are considered as a major driving force underlying natural speciation events. Understanding the molecular mechanisms resulting in hybrid incompatibilities between species is a central question for evolutionary biology and ecology.

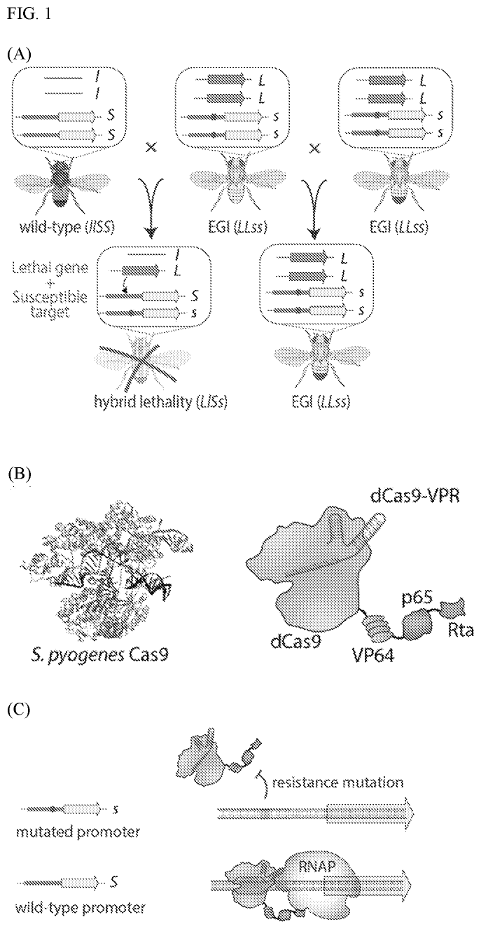

This disclosure describes a versatile and effective method for engineering DMIs in the lab to direct what amount to synthetic speciation events, referred to herein as engineered genetic incompatibility (EGI). In its most basic form, an EGI strain is made homozygous for a lethal effector gene and corresponding resistance allele. What separates EGI from described toxin/antitoxin systems is that the lethal effector allele is dominant, while the resistance allele is recessive. In other words, the EGI strain includes a haplosufficient lethal allele and a haploinsufficient resistance allele. Any outcrossing of the EGI strain with wild-type generates inviable hybrids, as the resulting heterozygotes contain the dominant lethal effector gene but only one copy of the recessive resistance allele (FIG. 1A). Unlike single locus, bi-allelic toxin-antitoxin systems, the EGI genotype in principle incurs no fitness penalty, as 100% of the offspring between similarly engineered EGI parents remain viable. The EGI approach leverages sequence-programmable transcription activators (PTAs) to drive lethal overexpression or ectopic expression of endogenous genes (FIG. 1B, 1C).

Thus is some embodiments, a cross between members of a wild-type population and an EGI strain can result in at least 50% lethality such as, for example, at least 80% lethality, at least 90% lethality, at least 95% lethality, at least 96% lethality, at least 97% lethality, at least 98% lethality, at least 99% lethality, at least 99.5% lethality, at least 99.9% lethality, at least 99.99% lethality, or at least 99.999% lethality. As used herein, the term “lethality” refers to the percentage of progeny that fail to develop to reproductive maturity, regardless of whether any individual progeny may survive.

In some embodiments, a cross between members of the EGI strain and other member of the same EGI strain can produce viable offspring. In some of these embodiments, a cross between two members of the same EGI strain can produce progeny with a viability of at least 75% such as, for example, at least 80%, at least 85%, at least 90%, at least 95%, at least 96%, at least 97%, at least 98%, or at least 99%. As used herein, the term “viability” refers to the percentage of progeny that survive to reproductive maturity.

In one exemplary application, the EGI approach was used to engineer extremely underdominant, ‘synthetic species’ of the model insect, Drosophila melanogaster. In this exemplary application, the strength and timing of hybrid lethality can be tuned based on genetic design. Further, multiple mutually-incompatible EGI genotypes can be created for the same target organism, allowing for the design of genetic biocontrol strategies that are robust in the face of genetic resistance.

### Lethal Overexpression of Endogenous Genes

The first goal was to empirically identify genes for which lethal overexpression or ectopic expression could be driven by a programmable transcription factor (PTA). To achieve this, a panel of engineered flies was created that were homozygous for the protein component of dCas9-based PTA. The engineered flies were mated to a second strain of flies that are engineered to be homozygous for sgRNA constructs. Lethal overexpression or ectopic expression were observed in the resulting hybrid progeny by tracking survival through developmental stages.

dCas9-VPR, composed of a catalytically inactive Cas9 fused to three transcriptional activation domains (VP64, p65, and Rta), was used as the transactivator. This construct has been reported to cause lethal gene activation in D. melanogaster heterozygotes. However, efficient lethal gene activation has not been previously shown using strains homozygous for dCas9-VPR. dCas9-VPR expression was constrained by replacing the promoter driving dCas9-VPR with a promoter from one of various developmental morphogens (pWg, pFoxo, pBam) or a truncated tubulin promoter (pTub). The constrained dCas9-VPR expression allows one to generate homozygous fly strains. Homozygous fly strains also were produced by expressing the evolved dXCas9-VPR transactivator from the truncated tubulin promoter.

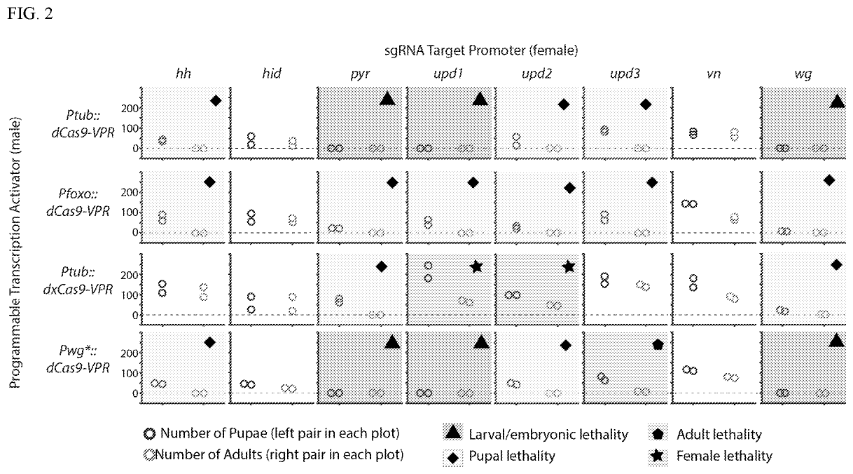

Homozygous dCas9-VPR strains were mated to strains homozygous for sgRNAs targeting several developmental morphogen genes (Hh, Hid, Pyr, Upd1, Upd2, Upd3, Wg, Vn). The parental flies were removed from mating vials after five days and the number of offspring surviving to pupal and adult life-stages were counted after 15 days (FIG. 2). Several crosses produced no surviving adult offspring in replicate experiments. Also, several hybrid incompatibility phenotypes were observed that depended on the combination of PTA and sgRNA used to drive overexpression or ectopic expression. Six crosses (FIG. 2, triangles) yielded little or no pupae, suggesting embryonic or larval lethality. The strongest early lethality was seen when pTub:dCas9-VPR or pWg:dCas9-VPR drove expression of the developmental morphogens Pyramus and Unpaired-1. Thirteen crosses (FIG. 2, diamonds) produced a strong pupal-lethal phenotype, with normal numbers of pupae forming, but no flies emerging as adults. One cross involving pWg:dCas9-VPR (FIG. 2, pentagons) produced a small number of surviving adults that were visibly deformed and died before they could reproduce. Finally, two crosses were observed with the pTub:dXCas9-VPR parent (FIG. 2, stars) that showed strong sex-ratio biasing, with predominantly (95%, upd1) or exclusively (100%, upd2) male survivors. These data were used to select a sub-set of putative target genes for constructing EGI flies, focusing on pyr, upd1, wg, and hh moving forward.

### Constructing EGI Strains

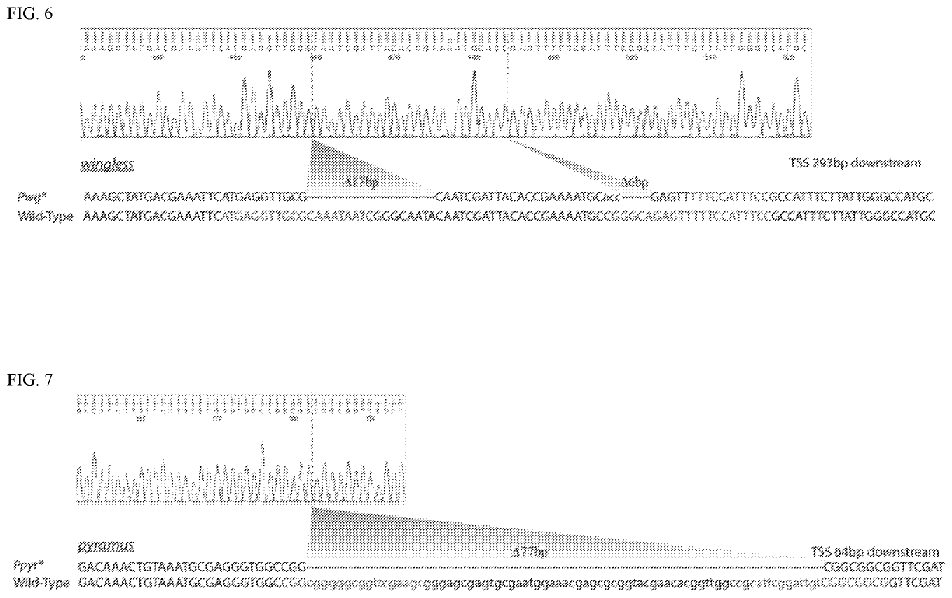

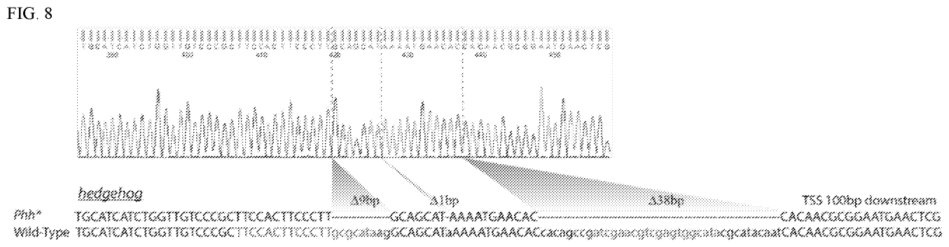

Recessive resistant alleles contain mutations to the sgRNA-binding sequences of target promoters to prevent lethal overexpression or ectopic expression (FIG. 1C). To generate viable promoter mutations, homozygous sgRNA-expressing strains were crossed to flies expressing germline Cas9. Offspring were crossed to balancers and F2 flies were screened for the presence of mutations via Sanger sequencing. Mutations were isolated that were homozygous viable and without any readily apparent phenotype for each of the target sites. Evidence for homozygous promoter mutations is shown in FIGS. 6-9.

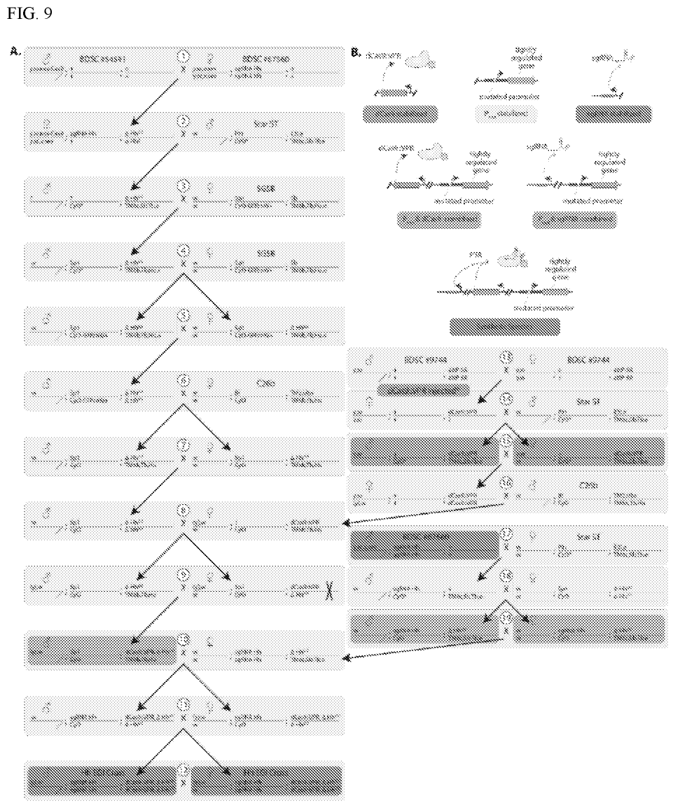

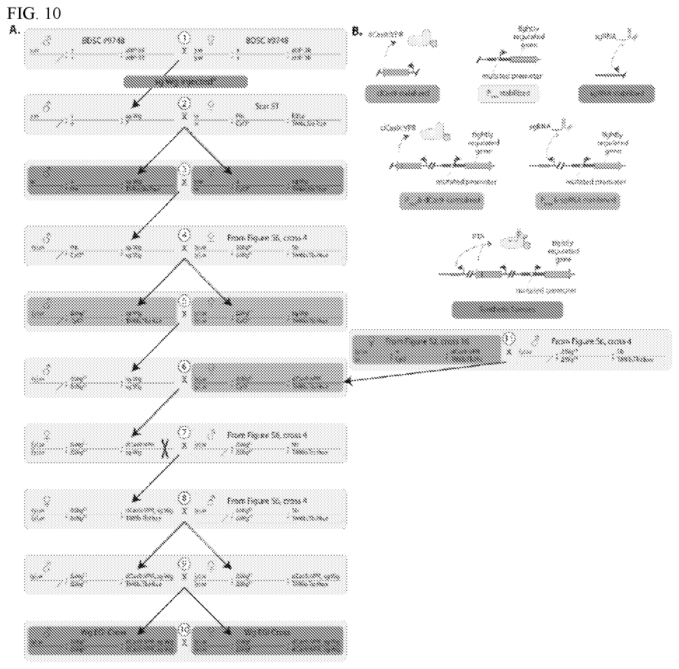

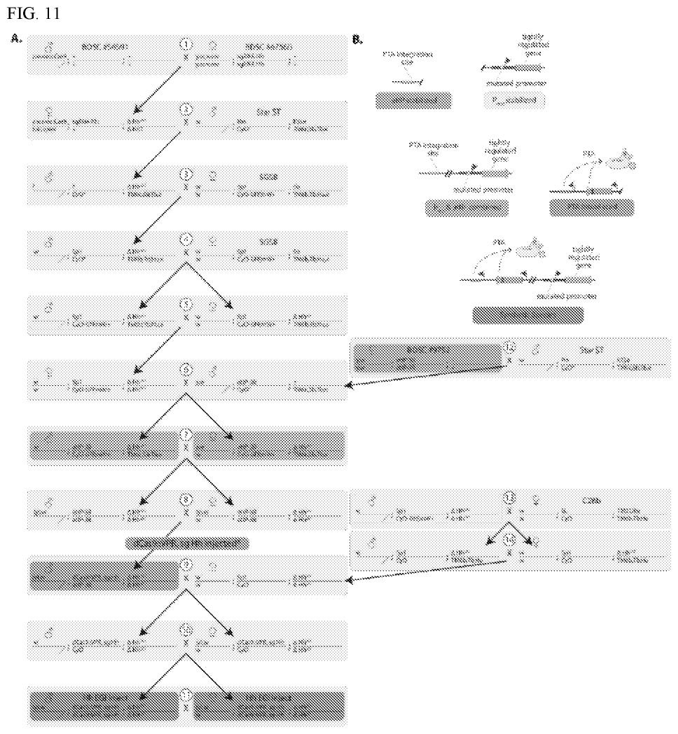

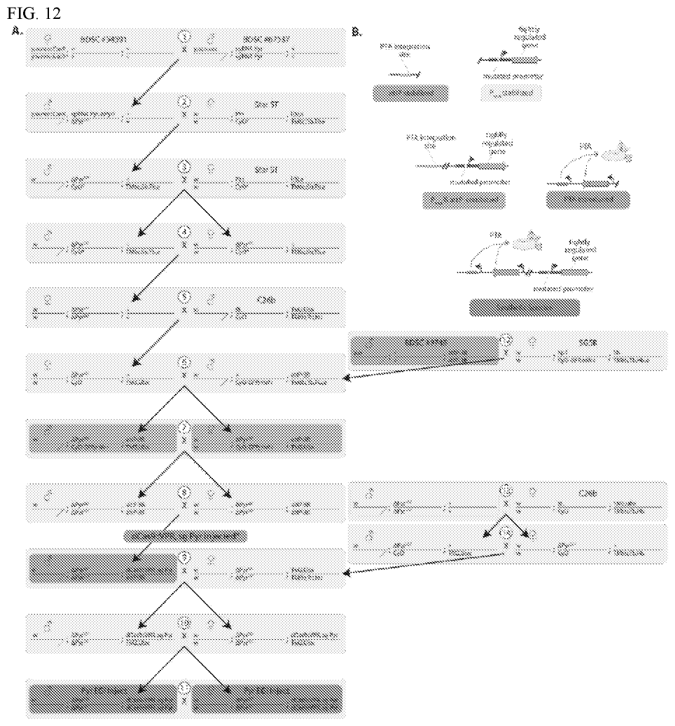

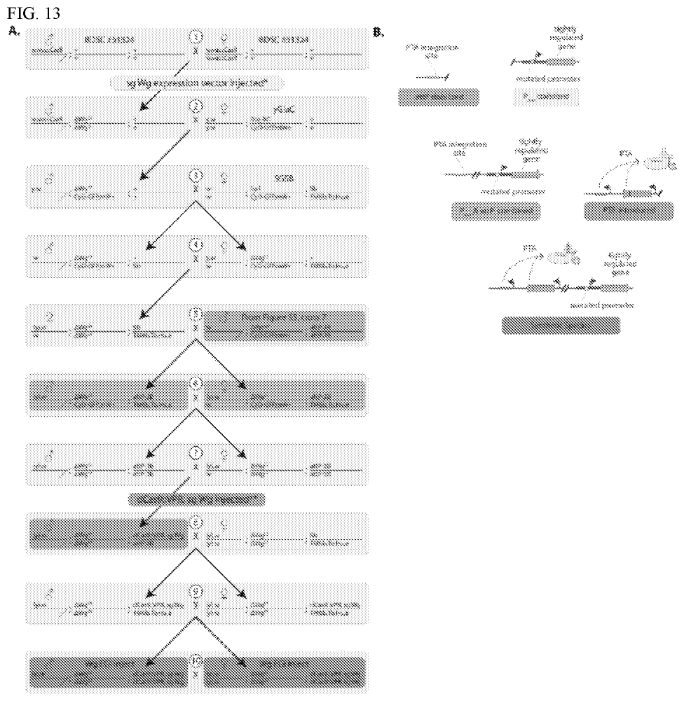

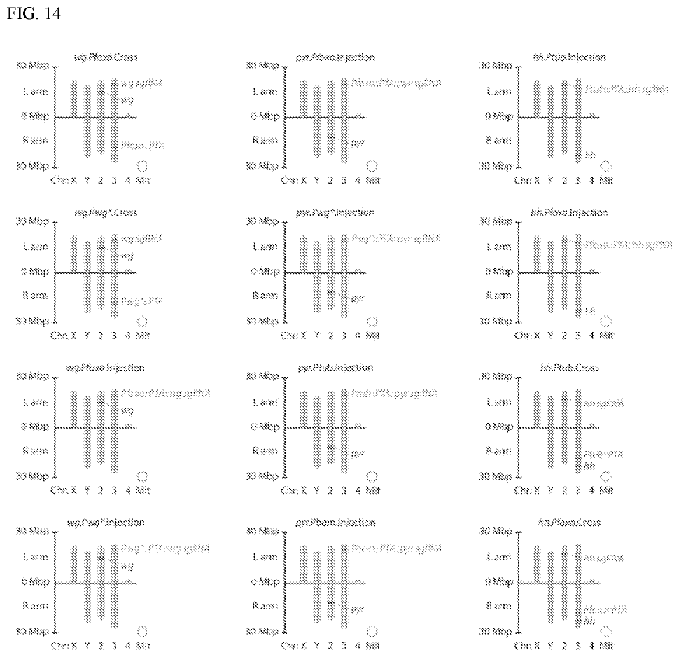

Both components were combined to create a full EGI genotype via one of two approaches. Both methods avoided passing through intermediate genotypes that contained an active PTA and a wild-type promoter sequence, as this would be lethal. The first method involved a series of crosses between flies containing PTA or sgRNA expression constructs that had already been characterized in FIG. 2. The second method involved re-injecting embryos from homozygous promoter mutant strain with a single plasmid containing expression constructs for both the dCas9-VPR and the sgRNA. The latter approach was more direct, requiring approximately half the number of crosses, but resulted in different chromosomal location for PTA expression compared to what was previously characterized. Using these two methods, a total of 15 unique EGI genotypes were produced. FIGS. 9-13 depict exemplary complete mating strategies used to assemble EGI components using each method. The specific number and order of matings varied slightly depending on chromosomal linkage of required components. Final chromosomal maps are shown in FIG. 14.

### Assessing Hybrid Incompatibility

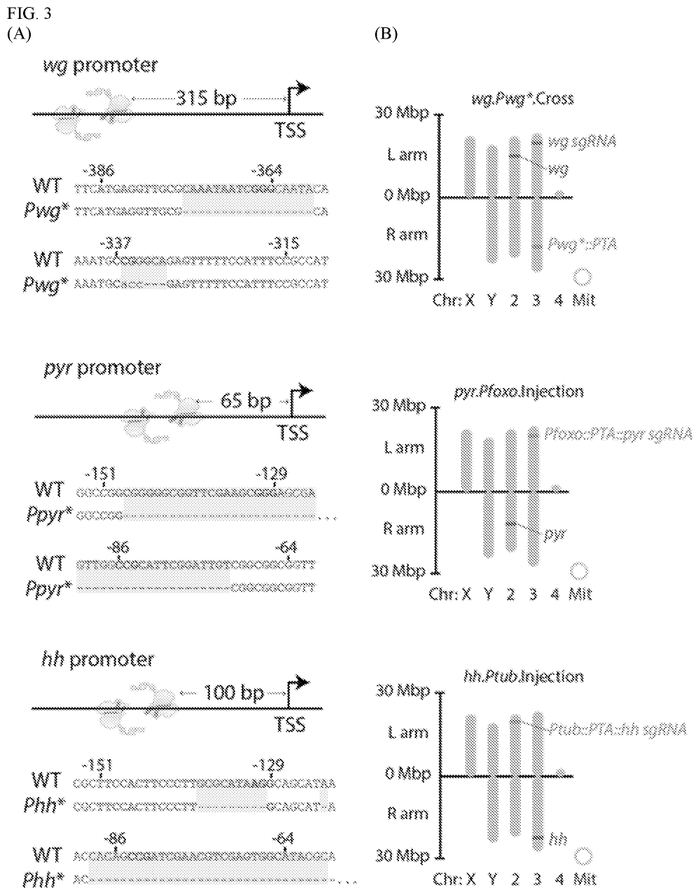

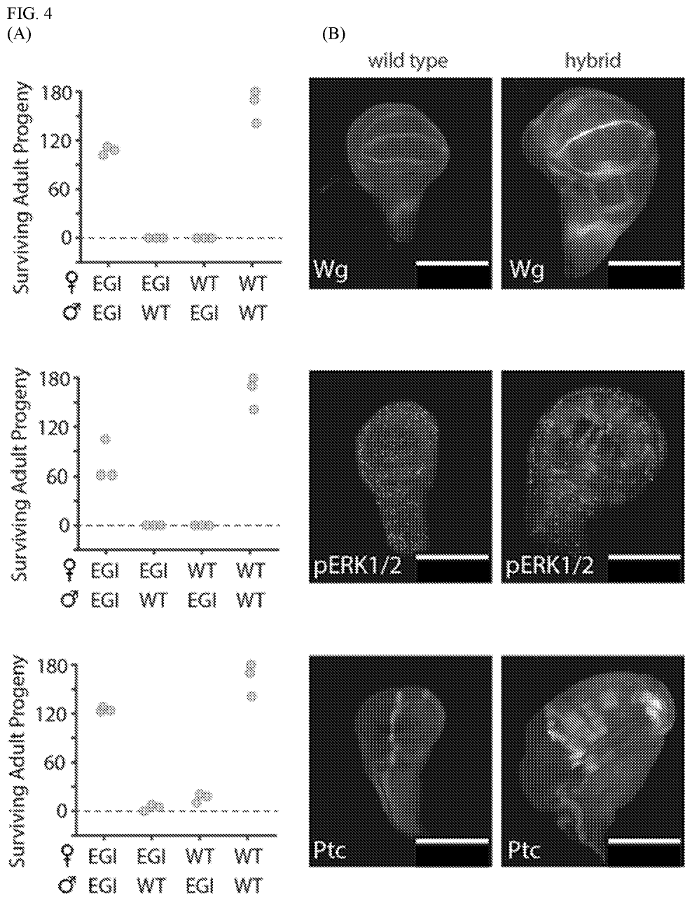

Candidate EGI strains were crossed to wild-type (Oregon R and w1118) to assess mating compatibility. While w1118 was the ‘wild-type’ starting point for our EGI engineering efforts, male w1118 flies have a previously reported mating phenotype. Oregon R males lack this mating phenotype and reproduce more efficiently. Intra-specific matings (male and female from the same EGI genotype) and EGI×wild-type matings were performed by combining three virgin females of one genotype with two males of another genotype. The number of pupal and adult progeny were counted after 15 days just as for the hybrid lethality screen described above. EGI strains that drove overexpression or ectopic expression of wingless or pyramus both showed full incompatibility, with no hybrids surviving to adulthood (FIG. 3 and FIG. 4). EGI strains with PTAs targeting the hedgehog promoter showed a marked underdominant phenotype, but not extreme underdominance. Approximately 10-13% of hybrid offspring from these crosses survived to adulthood. This is not surprising, as hedgehog was weaker than pyramus and wingless in the PTA×sgRNA crosses, yielding pupal lethality instead of larval lethality. The poor performance of the hedgehog EGI strains compared to the data in FIG. 2 may be the result of having only one sensitive (wild-type) promoter from which to drive lethal expression in the EGI×wild-type hybrids.

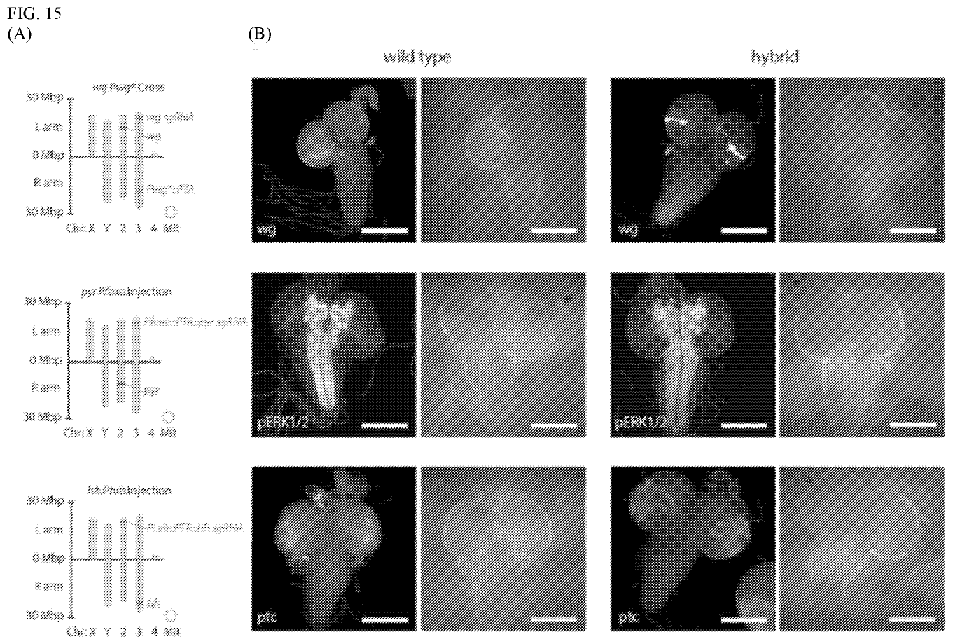

In order to confirm the mechanism of hybrid lethality, immunohistochemistry was performed on hybrid larva, staining for target gene overexpression or known signaling proteins that are down stream of the target genes. Clear evidence of ectopic expression was observed in hybrid larva but not larva from wild-type×wild-type or EGI×EGI crosses (FIG. 4B and FIG. 15).

Mutual Incompatibility Between EGI Strains with Distinct Genotypes

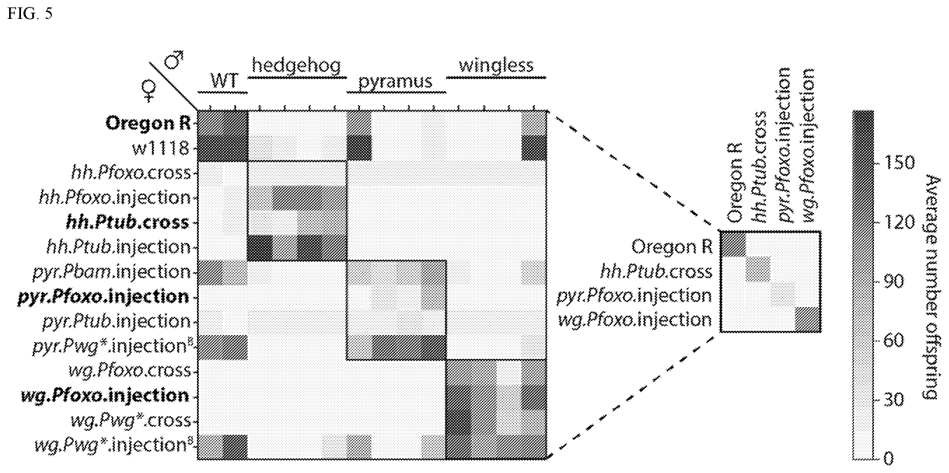

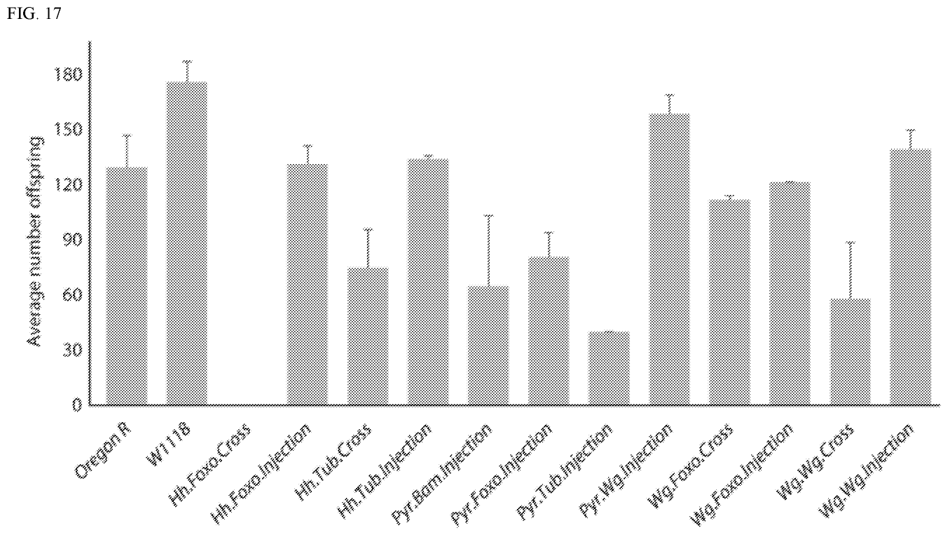

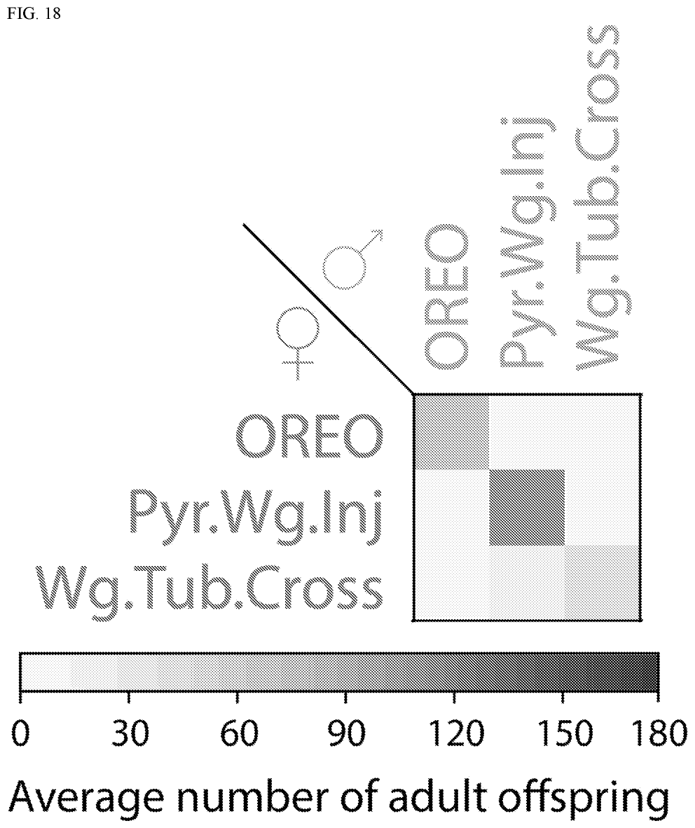

The method of generating species-like barriers to sexual reproduction described herein allows one to engineer not just one, but many EGI genotypes that are all incompatible with wild-type and/or with each other. To test this, a large cross-compatibility experiment was performed between 15 EGI genotypes. Each cross was performed bi-directionally (female of strain A to male of strain B and vice versa). The orthogonality plot in FIG. 5 shows expected compatibility results. Note that not all EGI×EGI′ genotypes were expected to be incompatible, as some differed only in the promoter driving the PTA or the chromosomal location of transgene constructs. The number of offspring obtained from intraspecific matings (i.e., like-kind matings) is represented on the diagonal of FIG. 5 and more explicitly as a bar graph in FIG. 17. Some of the EGI lines generated and tested in FIG. 5 were later found to contain some amount of balancer chromosomes in the population (marked with a subscript B on the vertical axis). The presence of a balancer chromosome explains the lake of incompatibility with wild-type, as these flies were essentially heterozygous for the EGI genotype. These two lines with balancer chromosomes were subsequently rebuilt (FIG. 18) and show 100% incompatibility with wildtype.

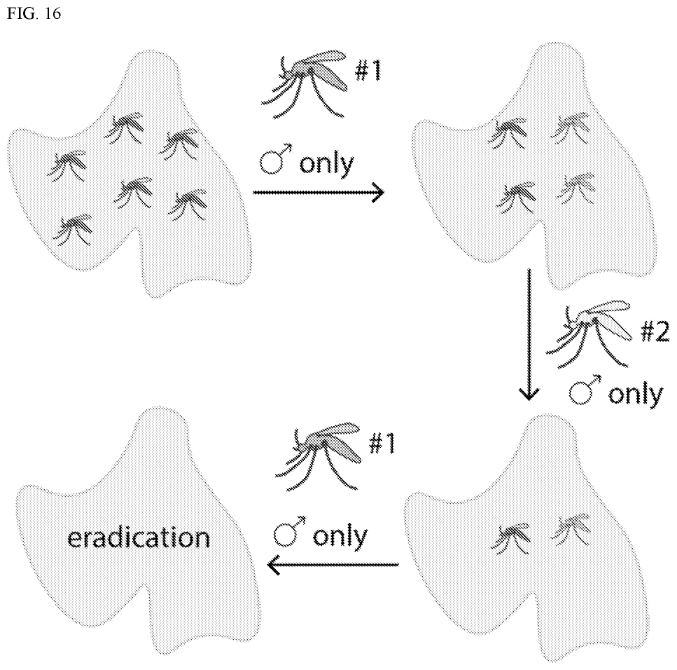

The ability to create mutually incompatible lines of EGI flies enables an iterative release paradigm for biocontrol applications that would mitigate the emergence of genetic escape mutants (FIG. 16). With two mutually incompatible EGI lines (i.e., incompatible with wildtype and also with each other), a release of a first population (illustrated as population #1 in FIG. 16) would initially suppress the wild-type population. Any surviving offspring that are resistant to the first population would persist and would inherit half of their DNA from the first population. This inherited genetic material would include alleles susceptible to the second release strain (illustrated as population #2 in FIG. 16). It would ensure that flies resistant to the first population are targetable by the next release of the second population. Any flies resistant to the second population would similarly inherit a susceptible allele for the first population so that this iterating release schedule could be repeated to avoid complications emerging from genetic resistance.

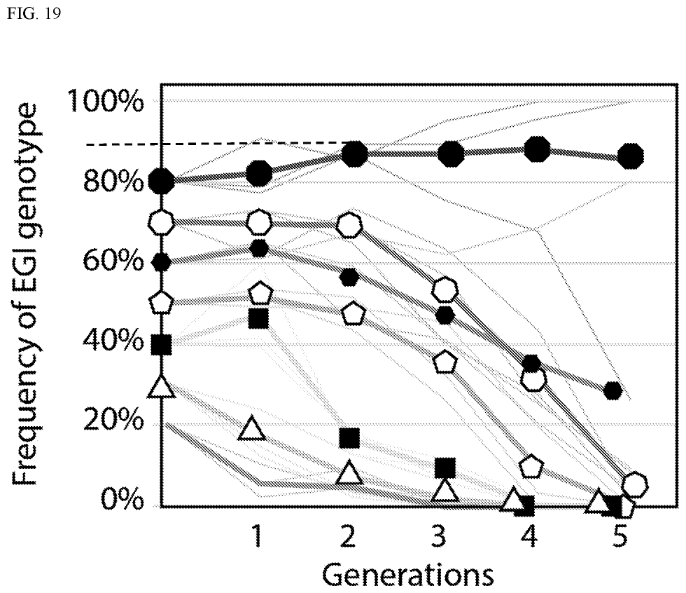

The ability of EGI to function as a threshold-dependent gene drive was tested (FIG. 19). EGI and wild-type flies (both males and females) were co-housed together in a single enclosure at different initial population compositions (from 20% EGI/80% wild-type to 80% EGI/20% wild-type). Threshold-dependent gene drives are bi-stable systems in which one genotype will go to fixation (100%) and one genotype will go to extinction (0%). With equal fitness, fecundity, and mating competitiveness, the expected threshold level was 50%. Our empirically measured threshold is ˜75%. This result is significant in that it demonstrates that EGI is capable of population replacement as a threshold-dependent gene drive, although this first generation of EGI gene drives has a higher than expected threshold.

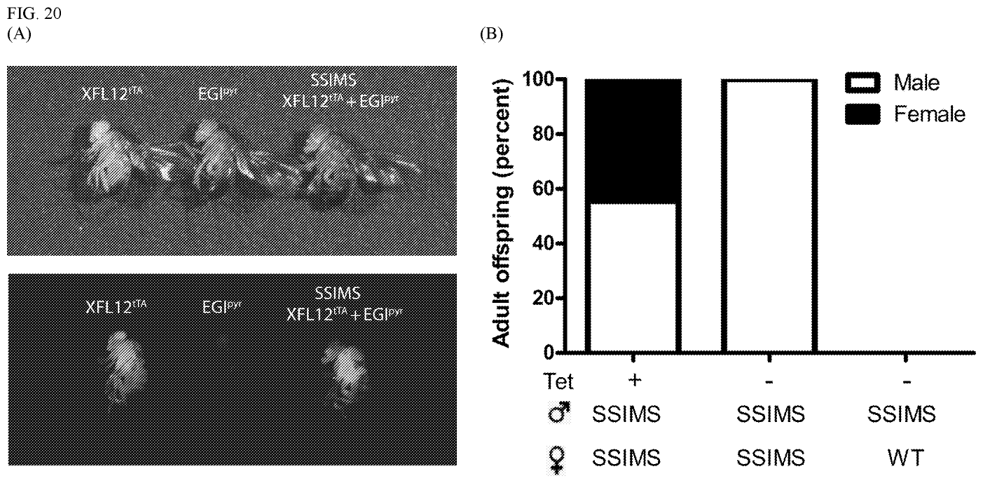

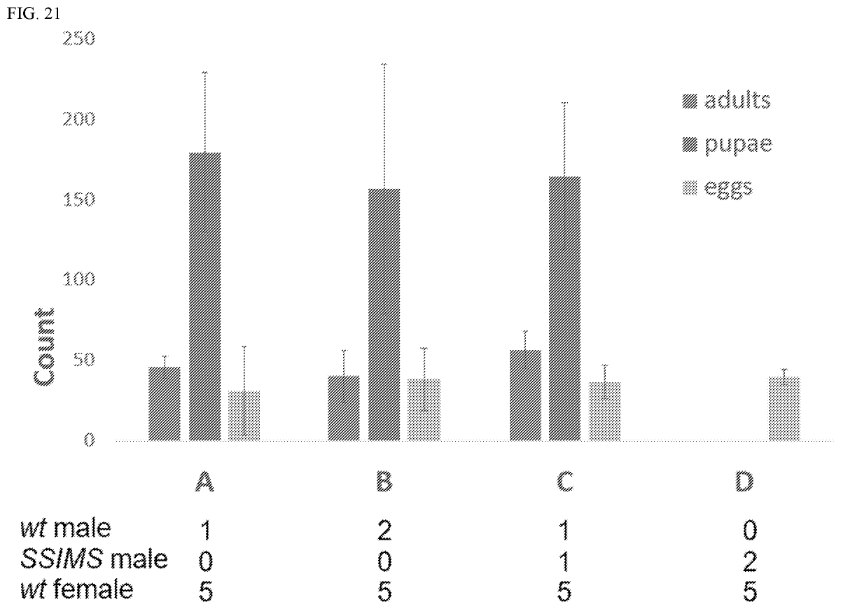

Next, the ability of EGI to work in scenarios similar to Sterile Insect Technique with an automated release was tested. To do this, the EGI genotype was combined with an automated sex-sorting construct in which females die in the absence of tetracycline. The combined EGI+Female Lethal genotype is called Self-Sorting Male Incompatibility System (SSIMS). The SSIMS flies could be created as stable lines (FIG. 20, FIG. 21). When cultured in the absence of tetracycline, only males survived (FIG. 20). When these males were crossed with wild-type females, none of the offspring were viable (FIG. 20, right panel). This incompatibility is also shown in the rightmost mating in FIG. 21, which produced no pupae or adults. When SSIMS males were mixed with wild-type males, the wild-type males outcompeted the SSIMS males (FIG. 21).

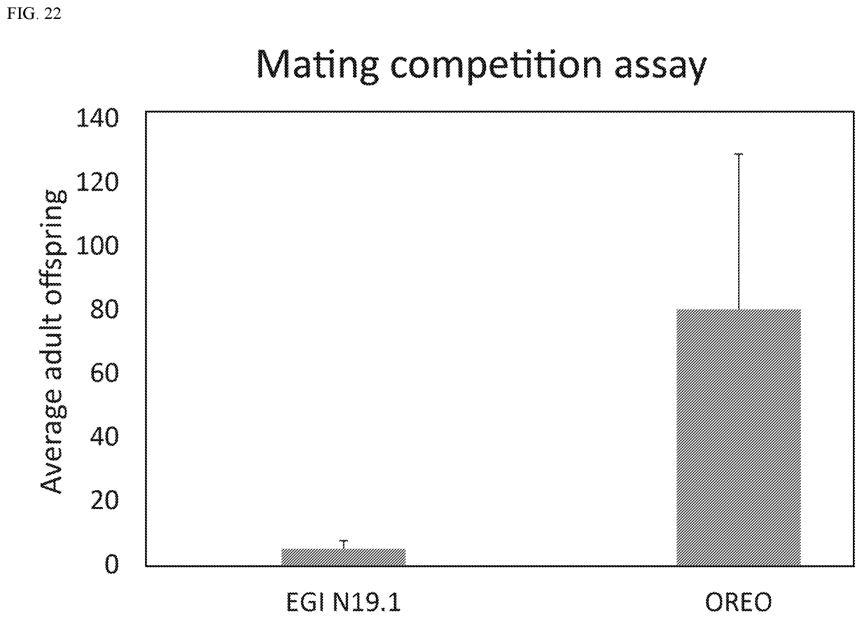

Finally, the ability of EGI males to compete with wildtype males for available mates was tested (FIG. 22) There may be some deficiency in the EGI males' ability to compete for mates or in the EGI females' fecundity. When Hh.Tub.Inj EGI flies were mated with themselves in the all by all cross, they produce a similar number of offspring as Oregon R flies mated to themselves. This variation in offspring count could also be caused by differences in media surface area as these tests were performed in bottles, which have approximately five times the surface area as vials. This added surface area results in higher carrying capacities of the container as there is less competition between larvae. The male mating competition phenotype explains why the threshold for a replacement drive (˜75%) is greater than the 50% expected if both strains mated equally well. This mating competition phenotype is not likely to be predictive of how each applied EGI strain will perform, as more find-tuned adjustment of dCas9 expression is likely to resolve the issue.

Thus, this disclosure describes a biocontainment system for multicellular organisms— i.e., species-like barriers to sexual reproduction in multicellular organisms. Generally, the biocontainment system produces an engineered genetic incompatibility (EGI) strain of a multicellular organism, in which the EGI strain has a haplosufficient lethal allele and a haploinsufficient resistance allele.

The successful implementation in a model multicellular organism (Drosophila melanogaster) confirms that this is a broadly applicable strategy for engineering reproductive barriers. Synthetic speciation has been previously described in D. melanogaster in which a non-essential transcription factor, glass, was knocked out and a glass-dependent lethal gene construct was introduced. While this approach uses a similar topology to the EGI approach (dominant lethal coupled to recessive resistance) described herein, the resulting flies were blind in the absence of glass, thus generating a noticeable phenotype that can deleteriously affect fitness. The use of programmable transcription activators in the EGI approach described herein to drive lethal overexpression or ectopic expression allows one to generate multiple EGI strains with no noticeable phenotypes aside from their hybrid incompatibility.

While described herein in the context of an exemplary embodiment in which the biocontainment system is introduced into D. melanogaster, the biocontainment system can be introduced into any multicellular organism. Exemplary plants into which the biocontainment system may be introduced can include, for example, a field crop (e.g., tobacco, corn, soybean, rice, etc.), a tree (e.g., poplar, rubber tree, etc.), or turfgrass (e.g. creeping bentgrass). Exemplary animals into which the biocontainment/biocontrol system may be introduced can include, for example, an insect (e.g., mosquito, tsetse fly, spotted-wing drosophila, olive fly, gypsy moth, codling moth, deer tick, etc.), a fish (e.g., salmon, carp, sea lamprey, etc.), a mammal (e.g., swine, a mouse, a rat, etc.), an amphibian (e.g., a cane toad, a bullfrog, etc.), a reptile (e.g., brown tree snake, etc.), a mollusk (e.g. zebra mussels), or a crustacean (e.g., rusty crayfish, etc.).

Generally, the biocontainment system includes a genetically-modified cell that includes a coding region whose expression results in death or infertility of the organism, a transcription regulatory region operably linked upstream of the coding region and having a silent mutation, and a polynucleotide that encodes a programmable transcription activator. The programmable transcription activator can be engineered to bind to the transcription regulatory region in the absence of the silent mutation, thereby initiating expression of the coding region in the absence of the silent mutation. Thus, in the absence of the silent mutation—i.e., if the organism is crossed with a wild type organism—the transcription activator initiates expression of the coding region and induces death or infertility of the organism. In the presence of the silent mutation—i.e., when the organism is crossed with another organism having the same biocontainment system— the transcription activator does not initiate expression of the coding region and the progeny organisms remain viable.

The biocontainment system can be designed so that expression of the coding region is overexpression or ectopic expression. As used herein, the term “overexpression” refers to a level of transcription of the coding region that is greater than that of a suitable wild-type control. Alternatively, or additionally, overexpression can refer to dysregulated expression, where the dynamic expression levels over time are perturbed such as, for example, a coding region that oscillates between an on-state and an off-state in wild-type that is constitutively in the on-state in the mutant. As used herein, “ectopic expression” refers to expression of the coding region in a tissue where it is normally silent. Expression of the coding region results in death or infertility of the organism in which the coding region is expressed.

Thus, the result of cross between an organism having the biocontainment system—i.e., are homozygous for the biocontainment system—and a wild-type organism results in progeny that are heterozygous for the biocontainment system, resulting in hybrid lethality/infertility.

As used herein, a “silent mutation” is a mutation in the DNA of the organism that does not significantly alter the phenotype of the organism outside of its effects within the context of the biocontainment system.

As used herein, the term “programmable transcription activator” refers to a transcription activator whose DNA binding specificity can be programmed. In the context of the biocontainment system described herein, the transcriptional activator is programmed to survey the genome of a cell for the wild-type transcription regulatory sequence that controls transcription of the target coding region, but does not bind to a variant of the transcription regulatory sequence that includes the silent mutation. While described herein in the context of an exemplary embodiment in which the programmable transcription activator is dCas9 fused to the activator domain VP64 and co-expressed with dCas9-VP64, other programmable transcription activators may be used in the biocontainment system. Exemplary alternative programmable transcription activators include, for example, fusions of dCas9, Cas9 (if combined with a short guide RNA), nuclease inactive CPF1, and TALEs to VP64, VP16, VPR, p65, Rta, EDLL, Ga14, TAD, SunTag or any combination thereof. In the case of RNA guided transcriptional regulators (e.g., dCas9-VP64), activation may be boosted by including aptamers in the RNA sequence which allow for the recruitment of aptamer binding protein such as, for example, transcription factor-fusions such as MS2/MCP, PCP, or COM fused to VP64, VP16, VPR, p65, Rta, and EDLL, Ga14, TAD or any combination thereof.

The coding region that is the target for expression can be any coding region whose expression causes death or infertility in a hybrid organism produced by a cross between an organism having the biocontainment system and an organism lacking the biocontainment system (e.g., a comparable wild-type organism or an organism having a different biocontainment system). In some cases, expression of the coding region can result in hybrid lethality—e.g., the progeny of the cross do not grow or are otherwise non-viable. In other cases, expression of the coding region can result in hybrid infertility—e.g., the progeny of the cross survive, but cannot produce progeny of their own.

In some cases, the coding region encodes a cytoskeletal polypeptide, an ER-Golgi vesicle polypeptide, an mRNA processing polypeptide, an electron transport polypeptide, a nuclear trafficking polypeptide, a chromosome segregation polypeptide, a spindle pole duplication polypeptide, an oxidative stress polypeptide, a cell-signaling polypeptide, a pro-apoptotic polypeptide, or a polypeptide controlling development (e.g., a developmental morphogen polypeptide).

In some cases, an organism may be engineered to include a second biocontainment system involving the programmed overexpression of a second coding region in the absence of a second silent mutation in the transcriptional regulatory region of the second coding region. The second biocontainment system can include a second programmable transcription activator. The second programmable transcription activator may be the same as the first programmable transcription activator in all respects other than the transcription regulatory sequence it is programmed to survey. In other cases, the second transcription activator may include different components that the programmable transcription activator of the first biocontainment system

Organisms possessing the biocontainment system—e.g., engineered genetic incompatibility (EGI organisms)—can be used in methods to suppress or replace a population of wild-type organisms such as, for example, pest organisms. As used herein, “suppression” of a wild-type population refers to reducing numbers of the target wild-type organism. For example, suppressing a wild-type population can include releasing EGI males repeatedly to compete with wild-type males to mate with wild-type females. The wild-type females that mate with EGI males will not have offspring and the next generation will be smaller. This can repeated each generation, and the population of wild-type organisms will continue to decline as the matings between wild-type females and wild-type males decline due to mating competition between the wild-type males and the EGI males. Eventually, the population will either be eradicated, or will be so small that only a modest release of EGI males will keep it suppressed to low levels.

As used herein, “replacement” of a wild-type population refers to changing from a wild-type population to a population of EGI organisms, with no decrease in total numbers. Replacement may be desirable in circumstances where one does not want to leave an unoccupied ecological niche. Population replacement can be used, for instance, to replace a population of mosquitos with an EGI version of the same species that has extra mutations that prevent it from spreading disease. To replace a population, one would release male and female EGI organisms. Wild-type organisms that mate with EGI organisms will not have offspring, so the wild-type population will be reduced. But EGI organisms that mate with other similar EGI organisms will produce offspring. Over generations, the EGI population can increase even without subsequent release of additional EGI organisms, but the EGI population can be augmented with additional releases of EGI organisms. As the percentage of EGI organisms in the population increases, wild-type organisms have more difficulty finding wild-type mates and, therefore, subsequent generations produce fewer and fewer wild-type organisms until, eventually, the wild-type population is replaced by a EGI population.

Thus, in another aspect, this disclosure describes a method of suppressing a population of a wild-type organisms. The method includes providing an engineered genetic incompatibility (EGI) strain of the wild-type organism and then mating members of the EGI strain of one sex with fertile adults of the opposite sex in the population of wild-type organisms. The EGI strain is engineered to possess a haplosufficient lethal allele and a haploinsufficient resistance allele so that progeny of wild-type×EGI crosses produce at least 50% lethality. As used in this context, “mating” members of the EGI strain and the wild-type population refers to any action that allows members of the EGI strain to mate. Thus, the term can include releasing members of the EGI strain into a natural environment in which a wild-type population of the organisms is known or suspected of inhabiting. The term also can include collecting members of a wild-type population and then combining members of the EGI strain and collected members of the wild-type population in a non-natural environment such as, for example, a vessel or enclosure of any kind.

The method of suppressing a population of the wild-type organisms can include multiple mating steps. That is, for example, the method can include multiple releases of members of the EGI strain into a natural environment. The timing and duration of multiple releases can be aligned with natural periods of mating behavior in the wild-type organism. The number of additional mating steps can be predetermined or can be continued until the wild-type population is suppressed to a desired degree. A degree to which the wild-type population is suppressed can depend, at least in part, on the particular wild-type organism whose population is being suppressed, the environmental effects of the wild-type organism, and/or the desired environmental effects of suppressing the population of the wild-type organism, although other factors can influence the degree to which the wild-type population is suppressed. Such factors are known to those of ordinary skill in the art.

In another aspect, this disclosure describes a method of replacing a population of wild-type organisms. The method includes providing an engineered genetic incompatibility (EGI) strain of the wild-type organism and mating the EGI strain with fertile adults in the population of wild-type organisms. The EGI strain is engineered to possess a haplosufficient lethal allele and a haploinsufficient resistance allele so that progeny of wild-type×EGI crosses produce at least 50% lethality and progeny of EGI×EGI crosses produce at least 75% viability. Here again, “mating” members of the EGI strain and the wild-type population refers to any action that allows members of the EGI strain to mate. Thus, the term can include releasing members of the EGI strain into a natural environment in which a wild-type population of the organisms is known or suspected of inhabiting. The term also can include collecting members of a wild-type population and then combining members of the EGI strain and collected members of the wild-type population in a non-natural environment such as, for example, a vessel or enclosure of any kind.

The method of replacing a population of the wild-type organisms with the EGI strain can include multiple mating steps. That is, for example, the method can include multiple releases of members of the EGI strain into a natural environment. The timing and duration of multiple releases can once again be aligned with natural periods of mating behavior in the wild-type organism. The number of additional mating steps can continue until the wild-type population is replaced by the EGI strain.

One difference between the method of suppressing a wild-type population and the method of replacing a wild-type population is in the members of the EGI strain that are mated with the members of the wild-type population. In the method of suppressing the wild-type population, only one sex of the EGI strain is mated with the wild-type strain. Matings between EGI organisms and wild-type organisms produce a certain degree of lethality—i.e., inviable progeny—and thereby decrease population count in the next generation. With multiple generations of matings involving EGI organisms and wild-type organisms, the overall population of the wild-type organisms decrease.

In the method to replace a wild-type population with an EGI population, both sexes of EGI organisms are mated with the wild-type organisms. Once again, matings between EGI organisms and wild-type organisms will produce a certain degree of lethality. Matings between EGI organisms and other EGI organisms of the same strain will be viable, however, and remain in the new heterogenous population. Each generation will include wild-type×EGI crosses that will decrease numbers of wild-type progeny in subsequent generations of the population, while EGI×EGI crosses will produce more EGI individuals, thereby providing more opportunity for EGI×wild-type crosses in the next generation. Eventually, the EGI strain numbers in the population will increase and wild-type numbers in the population will decrease so that the EGI strain wholly replaces the wild-type strain.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### Examples

### Plasmids

Plasmids expressing dCas9-VPR were constructed by Gibson assembly combining NotI linearized pMBO2744 attP vector backbone with dCas9-VPR PCR amplified from pAct:dCas9-VPR (Addgene #78898) and SV40 terminator for pH-Stinger (Bloomington Drosophila Stock Center, Bloomington, Ind.) to generate pMM7-6-1 (SEQ ID NO:1). Gibson assembly was used to clone 5′UTR and approximately 1.5 kb of promoter sequence into NotI linearized pMM7-6-1. Plasmids expressing dXCas9-VPR were constructed by introducing mutations into the dCas9 region predicted to improve activity to generate pMM7-9-3 (SEQ ID NO:6), which also has a NotI linearization site used for cloning promoter and 5′UTR sequences.

Plasmids expressing sgRNAs were generated by cloning annealed oligos into p{CFD4-3×P3::DsRed} (Addgene #86864).

Plasmids expressing both sgRNAs and dCas9-VPR were generated by assembling amplified sgRNA cassettes targeting pyr (Bloomington Drosophila Stock Center, Bloomington, Ind.; stock #67537), hh (Bloomington Drosophila Stock Center, Bloomington, Ind.; stock #67560) or wg (Bloomington Drosophila Stock Center, Bloomington, Ind.; stock #67545) genes into KpnI linearized plasmids pMM7-6-2 (SEQ ID NO:2), which includes the foxO1 promoter; pMM7-6-3 (SEQ ID NO:3), which includes the short tubulin promoter; pMM7-6-4 (SEQ ID NO:4), which includes the wingless (wg) promoter; or pMM7-6-5 (SEQ ID NO:5). The 12 different plasmid constructs are summarized in Table 1.

TABLE 1

| Plasmid | Construct | PTA promoter | PTA | sgRNA target |
|---|---|---|---|---|
| pAH1 | PTA-sgRNA | foxO1 | dCAS9-VPR | pyr |
| pAH2 | PTA-sgRNA | tubulin | dCAS9-VPR | pyr |
| pAH3 | PTA-sgRNA | wingless (wg) | dCAS9-VPR | pyr |
| pAH4 | PTA-sgRNA | barn | dCAS9-VPR | pyr |
| pAH5 | PTA-sgRNA | foxO1 | dCAS9-VPR | hh |
| pAH6 | PTA-sgRNA | tubulin | dCAS9-VPR | hh |
| pAH7 | PTA-sgRNA | wingless (wg) | dCAS9-VPR | hh |
| pAH8 | PTA-sgRNA | barn | dCAS9-VPR | hh |
| pAH9 | PTA-sgRNA | foxO1 | dCAS9-VPR | wg |
| pAH10 | PTA-sgRNA | tubulin | dCAS9-VPR | wg |
| pAH11 | PTA-sgRNA | wingless (wg) | dCAS9-VPR | wg |
| pAH12 | PTA-sgRNA | barn | dCAS9-VPR | wg |

Drosophila Stocks

Drosophila were maintained on standard cornmeal agar (NUTRI-FLY, Genesee Scientific Corp., El Cajon, Calif.). Experimental crosses were performed at 25° C. and 12 hour days. Existing Cas9 and sgRNA strains were obtained from the Bloomington Drosophila Stock Center (Bloomington, Ind.). All transgenic flies were generated via ΦC31 mediated integration targeted to attP landing sites. Embryo microinjections were performed by BestGene Inc. (Chino Hills, Ca).

### Mating Compatibility Tests

Genetic compatibility was assayed between parental stock homozygous for the PTA or sgRNA expression cassette (i.e. PTA-sgRNA) as well as between final EGI genotypes and wild-type (i.e., EGI testing). Test crosses were performed by crossing sexually-mature adult males to sexually-mature virgin females homozygous for their respective genotype at a ratio of 3:3 (PTA-sgRNA) or 2:3 (EGI testing). The adults were removed from the vials after five days and the offspring were counted alter fifteen days. Filled and empty pupal cases were counted towards the pupae total and adult males and females were counted towards the adult count. Independent mating compatibility tests were performed in duplicate (PTA-sgRNA) or triplicate (EGI tests).

Incompatibility crosses of Wg.Tub.Cross and Pyr.Wg.Inj

Additional incompatibility test crosses were performed for two EGI strains, Wg.Tub.Cross and Pyr.Wg.Inj. The Pyr.Wg.Inj strain used in the original manuscript was found to have balancer chromosomes and was thus not homozygous for the EGI components. Test crosses were performed as described immediately above, so these results are directly comparable to the all by all cross data performed in FIG. 5.

### Threshold Dependent Gene Drive Experiment

Populations were housed in 200 ml bottles. With the starting population size set to 100, males and females of EGI and wt (OregonR) strains were mixed at defined ratios representing the different thresholds. This starting population represents generation 1. For each generation adults were allowed to mate and lay eggs for five days, then collected and frozen for later analysis of % EGI in the population. On day 15, approximately 100-200 of the total progeny were randomly selected and placed in new bottles to seed the next generation. The remaining progeny were frozen for later analysis. The parents used to seed the bottle for each generation were analyzed by fluorescence microscopy to determine % EGI (RFP+) in the population. SSIMS male competition assay

Virgin wt females (3-6 day old) were mated with 3-4 day old wt or SSIMS males for 48 hours. After the 48-hour mating period, males were removed and females were transferred to hard-agar media for egg collection for 24 hours. Eggs laid were quantified the next day. Adults and pupae were quantified on day 12.

### Mating Competition Assay of Hh.Tub.Inj Vs Oregon R

A mating competitiveness assay was performed to determine the ability for males to compete and produce offspring when outnumbered 5-to-1. For the first bar (labeled EGI N19.1), one Hh.Tub.Inj male was added to a bottle with five Hh.Tub.Inj females and five Oregon R males. The adults were removed after five days and the number of adult offspring were counted on day 15 of the experiment. The bar depicts the average offspring from four replicates, with an error bar of one standard deviation. The second bar (labeled OREO) was the inverse cross—i.e., one OREO male was added to a bottle with five OREO females and five Hh.Tub.Inj males. Results are show in FIG. 22.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

Sequence Listing Free Text

SEQ ID NO: 1-pMM7-6-1
CCACNCACGTTTCGTAGTTGCTCTTTCGCTGTCTCCCACCCGCTNTCCGCAACACATTCACCTTTTGTTC
GACGACCNTNGGAGCGACTGTCGTTAGTTCCGCGCGATTCGGTTCGCTCAAATGGTTCCGAGTGGTTCAT
TTCGTCTCAATAGAAATTAGTAATAAATATTTGTATGTACAATTTATTTGCTCCAATATATTTGTATATA
TTTCCCTCACAGCTATATTTATTCTAATTTAATATTATGACTTTTTAAGGTAATTTTTTGTGACCTGTTC
GGAGTGATTAGCGTTACAATTTGAACTGAAAGTGACATCCAGTGTTTGTTCCTTGTGTAGATGCATCTCA
AAAAAATGGTGGGCATAATAGTGTTGTTTATATATATCAAAAATAACAACTATAATAATAAGAATACATT
TAATTTAGAAAATGCTTGGATTTCACTGGAACTAGGCTAGCATAACTTCGTATAATGTATGCTATACGAA
GTTATGCTAGCGGATCCGGGAATTGGGAATTCACGTAAGTACTGTCTGCAGCGTAAGCTTCGTACGTAGC
GGCCGCaatcttacaaaATGGACAAGAAGTACTCCATTGGGCTCGCTATCGGCACAAACAGCGTCGGCTG
GGCCGTCATTACGGACGAGTACAAGGTGCCGAGCAAAAAATTCAAAGTTCTGGGCAATACCGATCGCCAC
AGCATAAAGAAGAACCTCATTGGCGCCCTCCTGTTCGACTCCGGGGAGACGGCCGAAGCCACGCGGCTCA
AAAGAACAGCACGGCGCAGATATACCCGCAGAAAGAATCGGATCTGCTACCTGCAGGAGATCTTTAGTAA
TGAGATGGCTAAGGTGGATGACTCTTTCTTCCATAGGCTGGAGGAGTCCTTTTTGGTGGAGGAGGATAAA
AAGCACGAGCGCCACCCAATCTTTGGCAATATCGTGGACGAGGTGGCGTACCATGAAAAGTACCCAACCA
TATATCATCTGAGGAAGAAGCTTGTAGACAGTACTGATAAGGCTGACTTGCGGTTGATCTATCTCGCGCT
GGCGCATATGATCAAATTTCGGGGACACTTCCTCATCGAGGGGGACCTGAACCCAGACAACAGCGATGTC
GATAAACTCTTTATCCAACTGGTTCAGACTTACAATCAGCTTTTCGAAGAGAACCCGATCAACGCATCCG
GAGTTGACGCCAAAGCAATCCTGAGCGCTAGGCTGTCCAAATCCCGGCGGCTCGAAAACCTCATCGCACA
GCTCCCTGGGGAGAAGAAGAACGGCCTGTTTGGTAATCTTATCGCCCTGTCACTCGGGCTGACCCCCAAC
TTTAAATCTAACTTCGACCTGGCCGAAGATGCCAAGCTTCAACTGAGCAAAGACACCTACGATGATGATC
TCGACAATCTGCTGGCCCAGATCGGCGACCAGTACGCAGACCTTTTTTTGGCGGCAAAGAACCTGTCAGA
CGCCATTCTGCTGAGTGATATTCTGCGAGTGAACACGGAGATCACCAAAGCTCCGCTGAGCGCTAGTATG
ATCAAGCGCTATGATGAGCACCACCAAGACTTGACTTTGCTGAAGGCCCTTGTCAGACAGCAACTGCCTG
AGAAGTACAAGGAAATTTTCTTCGATCAGTCTAAAAATGGCTACGCCGGATACATTGACGGCGGAGCAAG
CCAGGAGGAATTTTACAAATTTATTAAGCCCATCTTGGAAAAAATGGACGGCACCGAGGAGCTGCTGGTA
AAGCTTAACAGAGAAGATCTGTTGCGCAAACAGCGCACTTTCGACAATGGAAGCATCCCCCACCAGATTC
ACCTGGGCGAACTGCACGCTATCCTCAGGCGGCAAGAGGATTTCTACCCCTTTTTGAAAGATAACAGGGA
AAAGATTGAGAAAATCCTCACATTTCGGATACCCTACTATGTAGGCCCCCTCGCCCGGGGAAATTCCAGA
TTCGCGTGGATGACTCGCAAATCAGAAGAGACCATCACTCCCTGGAACTTCGAGGAAGTCGTGGATAAGG
GGGCCTCTGCCCAGTCCTTCATCGAAAGGATGACTAACTTTGATAAAAATCTGCCTAACGAAAAGGTGCT
TCCTAAACACTCTCTGCTGTACGAGTACTTCACAGTTTATAACGAGCTCACCAAGGTCAAATACGTCACA
GAAGGGATGAGAAAGCCAGCATTCCTGTCTGGAGAGCAGAAGAAAGCTATCGTGGACCTCCTCTTCAAGA
CGAACCGGAAAGTTACCGTGAAACAGCTCAAAGAAGACTATTTCAAAAAGATTGAATGTTTCGACTCTGT
TGAAATCAGCGGAGTGGAGGATCGCTTCAACGCATCCCTGGGAACGTATCACGATCTCCTGAAAATCATT
AAAGACAAGGACTTCCTGGACAATGAGGAGAACGAGGACATTCTTGAGGACATTGTCCTCACCCTTACGT
TGTTTGAAGATAGGGAGATGATTGAAGAACGCTTGAAAACTTACGCTCATCTCTTCGACGACAAAGTCAT
GAAACAGCTCAAGAGGCGCCGATATACAGGATGGGGGCGGCTGTCAAGAAAACTGATCAATGGGATCCGA
GACAAGCAGAGTGGAAAGACAATCCTGGATTTTCTTAAGTCCGATGGATTTGCCAACCGGAACTTCATGC
AGTTGATCCATGATGACTCTCTCACCTTTAAGGAGGACATCCAGAAAGCACAAGTTTCTGGCCAGGGGGA
CAGTCTTCACGAGCACATCGCTAATCTTGCAGGTAGCCCAGCTATCAAAAAGGGAATACTGCAGACCGTT
AAGGTCGTGGATGAACTCGTCAAAGTAATGGGAAGGCATAAGCCCGAGAATATCGTTATCGAGATGGCCC
GAGAGAACCAAACTACCCAGAAGGGACAGAAGAACAGTAGGGAAAGGATGAAGAGGATTGAAGAGGGTAT
AAAAGAACTGGGGTCCCAAATCCTTAAGGAACACCCAGTTGAAAACACCCAGCTTCAGAATGAGAAGCTC
TACCTGTACTACCTGCAGAACGGCAGGGACATGTACGTGGATCAGGAACTGGACATCAATCGGCTCTCCG
ACTACGACGTGGCTGCTATCGTGCCCCAGTCTTTTCTCAAAGATGATTCTATTGATAATAAAGTGTTGAC
AAGATCCGATAAAGCTAGAGGGAAGAGTGATAACGTCCCCTCAGAAGAAGTTGTCAAGAAAATGAAAAAT
TATTGGCGGCAGCTGCTGAACGCCAAACTGATCACACAACGGAAGTTCGATAATCTGACTAAGGCTGAAC
GAGGTGGCCTGTCTGAGTTGGATAAAGCCGGCTTCATCAAAAGGCAGCTTGTTGAGACACGCCAGATCAC
CAAGCACGTGGCCCAAATTCTCGATTCACGCATGAACACCAAGTACGATGAAAATGACAAACTGATTCGA
GAGGTGAAAGTTATTACTCTGAAGTCTAAGCTGGTCTCAGATTTCAGAAAGGACTTTCAGTTTTATAAGG
TGAGAGAGATCAACAATTACCACCATGCGCATGATGCCTACCTGAATGCAGTGGTAGGCACTGCACTTAT
CAAAAAATATCCCAAGCTTGAATCTGAATTTGTTTACGGAGACTATAAAGTGTACGATGTTAGGAAAATG
ATCGCAAAGTCTGAGCAGGAAATAGGCAAGGCCACCGCTAAGTACTTCTTTTACAGCAATATTATGAATT
TTTTCAAGACCGAGATTACACTGGCCAATGGAGAGATTCGGAAGCGACCACTTATCGAAACAAACGGAGA
AACAGGAGAAATCGTGTGGGACAAGGGTAGGGATTTCGCGACAGTCCGGAAGGTCCTGTCCATGCCGCAG
GTGAACATCGTTAAAAAGACCGAAGTACAGACCGGAGGCTTCTCCAAGGAAAGTATCCTCCCGAAAAGGA
ACAGCGACAAGCTGATCGCACGCAAAAAAGATTGGGACCCCAAGAAATACGGCGGATTCGATTCTCCTAC
AGTCGCTTACAGTGTACTGGTTGTGGCCAAAGTGGAGAAAGGGAAGTCTAAAAAACTCAAAAGCGTCAAG
GAACTGCTGGGCATCACAATCATGGAGCGATCAAGCTTCGAAAAAAACCCCATCGACTTTCTCGAGGCGA
AAGGATATAAAGAGGTCAAAAAAGACCTCATCATTAAGCTTCCCAAGTACTCTCTCTTTGAGCTTGAAAA
CGGCCGGAAACGAATGCTCGCTAGTGCGGGCGAGCTGCAGAAAGGTAACGAGCTGGCACTGCCCTCTAAA
TACGTTAATTTCTTGTATCTGGCCAGCCACTATGAAAAGCTCAAAGGGTCTCCCGAAGATAATGAGCAGA
AGCAGCTGTTCGTGGAACAACACAAACACTACCTTGATGAGATCATCGAGCAAATAAGCGAATTCTCCAA
AAGAGTGATCCTCGCCGACGCTAACCTCGATAAGGTGCTTTCTGCTTACAATAAGCACAGGGATAAGCCC
ATCAGGGAGCAGGCAGAAAACATTATCCACTTGTTTACTCTGACCAACTTGGGCGCGCCTGCAGCCTTCA
AGTACTTCGACACCACCATAGACAGAAAGCGGTACACCTCTACAAAGGAGGTCCTGGACGCCACACTGAT
TCATCAGTCAATTACGGGGCTCTATGAAACAAGAATCGACCTCTCTCAGCTCGGTGGAGACAGCAGGGCT
GACCCCAAGAAGAAGAGGAAGGTGGAGGCCAGCGGTTCCGGACGGGCTGACGCATTGGACGATTTTGATC
TGGATATGCTGGGAAGTGACGCCCTCGATGATTTTGACCTTGACATGCTTGGTTCGGATGCCCTTGATGA
CTTTGACCTCGACATGCTCGGCAGTGACGCCCTTGATGATTTCGACCTGGACATGCTGATTAACTCTAGA
AGTTCCGGATCTCCGAAAAAGAAACGCAAAGTTGGTAGCCAGTACCTGCCCGACACCGACGACCGGCACC
GGATCGAGGAAAAGCGGAAGCGGACCTACGAGACATTCAAGAGCATCATGAAGAAGTCCCCCTTCAGCGG
CCCCACCGACCCTAGACCTCCACCTAGAAGAATCGCCGTGCCCAGCAGATCCAGCGCCAGCGTGCCAAAA
CCTGCCCCCCAGCCTTACCCCTTCACCAGCAGCCTGAGCACCATCAACTACGACGAGTTCCCTACCATGG
TGTTCCCCAGCGGCCAGATCTCTCAGGCCTCTGCTCTGGCTCCAGCCCCTCCTCAGGTGCTGCCTCAGGC
TCCTGCTCCTGCACCAGCTCCAGCCATGGTGTCTGCACTGGCTCAGGCACCAGCACCCGTGCCTGTGCTG
GCTCCTGGACCTCCACAGGCTGTGGCTCCACCAGCCCCTAAACCTACACAGGCCGGCGAGGGCACACTGT
CTGAAGCTCTGCTGCAGCTGCAGTTCGACGACGAGGATCTGGGAGCCCTGCTGGGAAACAGCACCGATCC
TGCCGTGTTCACCGACCTGGCCAGCGTGGACAACAGCGAGTTCCAGCAGCTGCTGAACCAGGGCATCCCT
GTGGCCCCTCACACCACCGAGCCCATGCTGATGGAATACCCCGAGGCCATCACCCGGCTCGTGACAGGCG
CTCAGAGGCCTCCTGATCCAGCTCCTGCCCCTCTGGGAGCACCAGGCCTGCCTAATGGACTGCTGTCTGG
CGACGAGGACTTCAGCTCTATCGCCGATATGGATTTCTCAGCCTTGCTGGGCTCTGGCAGCGGCAGCCGG
GATTCCAGGGAAGGGATGTTTTTGCCGAAGCCTGAGGCCGGCTCCGCTATTAGTGACGTGTTTGAGGGCC
GCGAGGTGTGCCAGCCAAAACGAATCCGGCCATTTCATCCTCCAGGAAGTCCATGGGCCAACCGCCCACT
CCCCGCCAGCCTCGCACCAACACCAACCGGTCCAGTACATGAGCCAGTCGGGTCACTGACCCCGGCACCA
GTCCCTCAGCCACTGGATCCAGCGCCCGCAGTGACTCCCGAGGCCAGTCACCTGTTGGAGGATCCCGATG
AAGAGACGAGCCAGGCTGTCAAAGCCCTTCGGGAGATGGCCGATACTGTGATTCCCCAGAAGGAAGAGGC
TGCAATCTGTGGCCAAATGGACCTTTCCCATCCGCCCCCAAGGGGCCATCTGGATGAGCTGACAACCACA
CTTGAGTCCATGACCGAGGATCTGAACCTGGACTCACCCCTGACCCCGGAATTGAACGAGATTCTGGATA
CCTTCCTGAACGACGAGTGCCTCTTGCATGCCATGCATATCAGCACAGGACTGTCCATCTTCGACACATC
TCTGTTTTGAccgactctagatcataatcagccataccacatttgtagaggttttacttgctttaaaaaa
cctcccacacctccccctgaacctgaaacataaaatgaatgcaattgttgttgttaacttgtttattgca
gcttataatggttacaaataaagcaatagcatcacaaatttcacaaataaagcatttttttcactgcatt
ctagttgtggtttgcccaaactcatcaatgtatcttaGCGGCTCGAGGGTACCTCTAGAGATCCACTAGT
GTCGACGATGTAGGTCACGGTCTCGAAGCCGCGGTGCGGGTGCCAGGGCGTGCCCTTGGGCTCCCCGGGC
GCGTACTCCACCTCACCCATCTGGTCCATCATGATGAACGGGTCGAGGTGGCGGTAGTTGATCCCGGCGA
ACGCGCGGCGCACCGGGAAGCCCTCGCCCTCGAAACCGCTGGGCGCGGTGGTCACGGTGAGCACGGGACG
TGCGACGGCGTCGGCGGGTGCGGATACGCGGGGCAGCGTCAGCGGGTTCTCGACGGTCACGGCGGGCATG
TCGACACTA
SEQ ID NO: 2-pMM7-6-2
CCACNCACGTTTCGTAGTTGCTCTTTCGCTGTCTCCCACCCGCTNTCCGCAACACATTCACCTTTTGTTC
GACGACCNTNGGAGCGACTGTCGTTAGTTCCGCGCGATTCGGTTCGCTCAAATGGTTCCGAGTGGTTCAT
TTCGTCTCAATAGAAATTAGTAATAAATATTTGTATGTACAATTTATTTGCTCCAATATATTTGTATATA
TTTCCCTCACAGCTATATTTATTCTAATTTAATATTATGACTTTTTAAGGTAATTTTTTGTGACCTGTTC
GGAGTGATTAGCGTTACAATTTGAACTGAAAGTGACATCCAGTGTTTGTTCCTTGTGTAGATGCATCTCA
AAAAAATGGTGGGCATAATAGTGTTGTTTATATATATCAAAAATAACAACTATAATAATAAGAATACATT
TAATTTAGAAAATGCTTGGATTTCACTGGAACTAGGCTAGCATAACTTCGTATAATGTATGCTATACGAA
GTTATGCTAGCGGATCCGGGAATTGGGAATTCACGTAAGTACTGTCTGCAGCGTAAGCTTCGTACGTAGC
gtcaaatttggttgtgattacgagacggagaccgagacggcgacgacagttagccattcgccacgcgcca
acgcaaatgaaacgctctatacatatttttgtatattttctgtttttttttgccgctgacaattatgatc
aagtattagctggcgatagctgaaacgtctgtgtaatttcaatggaatggaatgggaagtgggcggccta
ttgatacactgctcgagtgattttaacttttatctgatcattcaaacgcataaattagtcttgagaactt
caattcatttgatactccagttaacatgctatttacatgctcatttaaatggtagtagtgatttataagc
ccacttccagatggaacttacctataccaacgtgttacttatcgttcttaagccaacttaatagcattct
aaaatatatatgtatcttttggcggacttatcttcttgttgttctcgcattccaaaatctctatgtacat
gcaaacttttattgtcataactcgggactttgcagactttgaggcctatttaatagagctataatcttac
aacaaaaaaaaactaaaagagctttttaagcaataaaaatattctgaaaaattacaaattaacaaaaaat
tacccaatgaagcctgcaaatttgaaatctttaagatcctagatatgccaagatgcaccctaaagtcctt
aactcatctccttggctcgtttctaatcccccctctcgagggatcgagacgatcgcatcgggtcggtctt
taagtttggatgatccataaactgttggtttctccgtcctcagcgtctagacttcattagccgtgtaatg
ttgcggaatttatgtggcaggcacattaaaataacaccgatacacactctcatggacgcgaacgtgtgta
caagtatagagatatcgggcctaggcgaaaaatgaaattaaaaaaaaaaaaaaaactggcaccgggaggg
gcttatttttcggtggtcggggatgcgggggactttgaccataaaatacatgctcccaaaaagctcgcac
actgcaagagatgcggggcacttctgagtcccatattcatatgcacaaatgtgcattgctggcattatca
gtagaatgcaatttcgggaaattttccatcgcatcacgagacaatgaacgtaagagagaaatggagcctc
aaagagggagggagagagagagcttgagtgaacgagcgagcgacaatcgcgagataacggctgccttatc
agcaatgccgaccgccccatcaccccacccaaaacgcccaaccaccacccaccgccgccgttccctttcc
tccatcgtcgagaatttcgagttcagagcagcgcgaccgaaatgaaaagaaacaatttaaattccaaatg
tataaaataggtaaactatggcttttatttattaatattgacgggggcacaaaggcggtcacctcaatag
tgaataacatgttttttataatgaatacttttcaaattgttattaatatgcaatgacgtcttaaatgttt
cactgcagctgaactttattcttttcattaaaacagtcacccgttattaaaaataaatagattaagtttt
atattattaaatttgtaagtattgaaacaattccttttttattttatatgaattatcatttagttggggt
taatatcccttaaagagagaaatttgtatgctttaagatttaaaatatctattgcatttatagctatagc
tataacttctcttatttcacgcagaaaatactcaaataaaacatatcgatttggcataccccactaattt
tttggccccaagtgtgtgagagtgtgtgaggcgaagcgcgccacaaacataaaaaagcggtgaagtgagc
ggttgtggaacgtgagtggatgctaagagcaagctctcacatacgcggacataggtcgcacacacacacg
cacagaccgcctttttgcgccgccgaaacgaacacttttacgaaggcgacggcgaatcagtttcagttgt
cagttcgcatccaactagaaagcagttaacgagtagtctgtgttttttcgcttgcggttaaaagccacga
ggtcgttcatcgttcatcgttttccttttcaacttcaagcaaagcaaatataaaccaatgcaaaaaacgc
agtgatcttttgaggcccaaatcgtttggggccgaacaccgttgattctaaaacgcaaatgtagaaacaa
atcaagaaagtggaaaataaatatgtttcgctttcaaaacatgtgaatgtgccgaactcaaaactgaaac
gtagaaggaacgcgttcgttttttacatacgacaatcgtataaaataagagaaaagctccaaaacgtatt
aaatagcgatgcttggatgatcttcgtagcagtcacgttgtacatacaaatacatacatatgtacctact
atatggcacataaaatacgttacgcacactagtggcgaataaaaagcgaattggaGCaatcttacaaaAT
GGACAAGAAGTACTCCATTGGGCTCGCTATCGGCACAAACAGCGTCGGCTGGGCCGTCATTACGGACGAG
TACAAGGTGCCGAGCAAAAAATTCAAAGTTCTGGGCAATACCGATCGCCACAGCATAAAGAAGAACCTCA
TTGGCGCCCTCCTGTTCGACTCCGGGGAGACGGCCGAAGCCACGCGGCTCAAAAGAACAGCACGGCGCAG
ATATACCCGCAGAAAGAATCGGATCTGCTACCTGCAGGAGATCTTTAGTAATGAGATGGCTAAGGTGGAT
GACTCTTTCTTCCATAGGCTGGAGGAGTCCTTTTTGGTGGAGGAGGATAAAAAGCACGAGCGCCACCCAA
TCTTTGGCAATATCGTGGACGAGGTGGCGTACCATGAAAAGTACCCAACCATATATCATCTGAGGAAGAA
GCTTGTAGACAGTACTGATAAGGCTGACTTGCGGTTGATCTATCTCGCGCTGGCGCATATGATCAAATTT
CGGGGACACTTCCTCATCGAGGGGGACCTGAACCCAGACAACAGCGATGTCGATAAACTCTTTATCCAAC
TGGTTCAGACTTACAATCAGCTTTTCGAAGAGAACCCGATCAACGCATCCGGAGTTGACGCCAAAGCAAT
CCTGAGCGCTAGGCTGTCCAAATCCCGGCGGCTCGAAAACCTCATCGCACAGCTCCCTGGGGAGAAGAAG
AACGGCCTGTTTGGTAATCTTATCGCCCTGTCACTCGGGCTGACCCCCAACTTTAAATCTAACTTCGACC
TGGCCGAAGATGCCAAGCTTCAACTGAGCAAAGACACCTACGATGATGATCTCGACAATCTGCTGGCCCA
GATCGGCGACCAGTACGCAGACCTTTTTTTGGCGGCAAAGAACCTGTCAGACGCCATTCTGCTGAGTGAT
ATTCTGCGAGTGAACACGGAGATCACCAAAGCTCCGCTGAGCGCTAGTATGATCAAGCGCTATGATGAGC
ACCACCAAGACTTGACTTTGCTGAAGGCCCTTGTCAGACAGCAACTGCCTGAGAAGTACAAGGAAATTTT
CTTCGATCAGTCTAAAAATGGCTACGCCGGATACATTGACGGCGGAGCAAGCCAGGAGGAATTTTACAAA
TTTATTAAGCCCATCTTGGAAAAAATGGACGGCACCGAGGAGCTGCTGGTAAAGCTTAACAGAGAAGATC
TGTTGCGCAAACAGCGCACTTTCGACAATGGAAGCATCCCCCACCAGATTCACCTGGGCGAACTGCACGC
TATCCTCAGGCGGCAAGAGGATTTCTACCCCTTTTTGAAAGATAACAGGGAAAAGATTGAGAAAATCCTC
ACATTTCGGATACCCTACTATGTAGGCCCCCTCGCCCGGGGAAATTCCAGATTCGCGTGGATGACTCGCA
AATCAGAAGAGACCATCACTCCCTGGAACTTCGAGGAAGTCGTGGATAAGGGGGCCTCTGCCCAGTCCTT
CATCGAAAGGATGACTAACTTTGATAAAAATCTGCCTAACGAAAAGGTGCTTCCTAAACACTCTCTGCTG
TACGAGTACTTCACAGTTTATAACGAGCTCACCAAGGTCAAATACGTCACAGAAGGGATGAGAAAGCCAG
CATTCCTGTCTGGAGAGCAGAAGAAAGCTATCGTGGACCTCCTCTTCAAGACGAACCGGAAAGTTACCGT
GAAACAGCTCAAAGAAGACTATTTCAAAAAGATTGAATGTTTCGACTCTGTTGAAATCAGCGGAGTGGAG
GATCGCTTCAACGCATCCCTGGGAACGTATCACGATCTCCTGAAAATCATTAAAGACAAGGACTTCCTGG
ACAATGAGGAGAACGAGGACATTCTTGAGGACATTGTCCTCACCCTTACGTTGTTTGAAGATAGGGAGAT
GATTGAAGAACGCTTGAAAACTTACGCTCATCTCTTCGACGACAAAGTCATGAAACAGCTCAAGAGGCGC
CGATATACAGGATGGGGGCGGCTGTCAAGAAAACTGATCAATGGGATCCGAGACAAGCAGAGTGGAAAGA
CAATCCTGGATTTTCTTAAGTCCGATGGATTTGCCAACCGGAACTTCATGCAGTTGATCCATGATGACTC
TCTCACCTTTAAGGAGGACATCCAGAAAGCACAAGTTTCTGGCCAGGGGGACAGTCTTCACGAGCACATC
GCTAATCTTGCAGGTAGCCCAGCTATCAAAAAGGGAATACTGCAGACCGTTAAGGTCGTGGATGAACTCG
TCAAAGTAATGGGAAGGCATAAGCCCGAGAATATCGTTATCGAGATGGCCCGAGAGAACCAAACTACCCA
GAAGGGACAGAAGAACAGTAGGGAAAGGATGAAGAGGATTGAAGAGGGTATAAAAGAACTGGGGTCCCAA
ATCCTTAAGGAACACCCAGTTGAAAACACCCAGCTTCAGAATGAGAAGCTCTACCTGTACTACCTGCAGA
ACGGCAGGGACATGTACGTGGATCAGGAACTGGACATCAATCGGCTCTCCGACTACGACGTGGCTGCTAT
CGTGCCCCAGTCTTTTCTCAAAGATGATTCTATTGATAATAAAGTGTTGACAAGATCCGATAAAGCTAGA
GGGAAGAGTGATAACGTCCCCTCAGAAGAAGTTGTCAAGAAAATGAAAAATTATTGGCGGCAGCTGCTGA
ACGCCAAACTGATCACACAACGGAAGTTCGATAATCTGACTAAGGCTGAACGAGGTGGCCTGTCTGAGTT
GGATAAAGCCGGCTTCATCAAAAGGCAGCTTGTTGAGACACGCCAGATCACCAAGCACGTGGCCCAAATT
CTCGATTCACGCATGAACACCAAGTACGATGAAAATGACAAACTGATTCGAGAGGTGAAAGTTATTACTC
TGAAGTCTAAGCTGGTCTCAGATTTCAGAAAGGACTTTCAGTTTTATAAGGTGAGAGAGATCAACAATTA
CCACCATGCGCATGATGCCTACCTGAATGCAGTGGTAGGCACTGCACTTATCAAAAAATATCCCAAGCTT
GAATCTGAATTTGTTTACGGAGACTATAAAGTGTACGATGTTAGGAAAATGATCGCAAAGTCTGAGCAGG
AAATAGGCAAGGCCACCGCTAAGTACTTCTTTTACAGCAATATTATGAATTTTTTCAAGACCGAGATTAC
ACTGGCCAATGGAGAGATTCGGAAGCGACCACTTATCGAAACAAACGGAGAAACAGGAGAAATCGTGTGG
GACAAGGGTAGGGATTTCGCGACAGTCCGGAAGGTCCTGTCCATGCCGCAGGTGAACATCGTTAAAAAGA
CCGAAGTACAGACCGGAGGCTTCTCCAAGGAAAGTATCCTCCCGAAAAGGAACAGCGACAAGCTGATCGC
ACGCAAAAAAGATTGGGACCCCAAGAAATACGGCGGATTCGATTCTCCTACAGTCGCTTACAGTGTACTG
GTTGTGGCCAAAGTGGAGAAAGGGAAGTCTAAAAAACTCAAAAGCGTCAAGGAACTGCTGGGCATCACAA
TCATGGAGCGATCAAGCTTCGAAAAAAACCCCATCGACTTTCTCGAGGCGAAAGGATATAAAGAGGTCAA
AAAAGACCTCATCATTAAGCTTCCCAAGTACTCTCTCTTTGAGCTTGAAAACGGCCGGAAACGAATGCTC
GCTAGTGCGGGCGAGCTGCAGAAAGGTAACGAGCTGGCACTGCCCTCTAAATACGTTAATTTCTTGTATC
TGGCCAGCCACTATGAAAAGCTCAAAGGGTCTCCCGAAGATAATGAGCAGAAGCAGCTGTTCGTGGAACA
ACACAAACACTACCTTGATGAGATCATCGAGCAAATAAGCGAATTCTCCAAAAGAGTGATCCTCGCCGAC
GCTAACCTCGATAAGGTGCTTTCTGCTTACAATAAGCACAGGGATAAGCCCATCAGGGAGCAGGCAGAAA
ACATTATCCACTTGTTTACTCTGACCAACTTGGGCGCGCCTGCAGCCTTCAAGTACTTCGACACCACCAT
AGACAGAAAGCGGTACACCTCTACAAAGGAGGTCCTGGACGCCACACTGATTCATCAGTCAATTACGGGG
CTCTATGAAACAAGAATCGACCTCTCTCAGCTCGGTGGAGACAGCAGGGCTGACCCCAAGAAGAAGAGGA
AGGTGGAGGCCAGCGGTTCCGGACGGGCTGACGCATTGGACGATTTTGATCTGGATATGCTGGGAAGTGA
CGCCCTCGATGATTTTGACCTTGACATGCTTGGTTCGGATGCCCTTGATGACTTTGACCTCGACATGCTC
GGCAGTGACGCCCTTGATGATTTCGACCTGGACATGCTGATTAACTCTAGAAGTTCCGGATCTCCGAAAA
AGAAACGCAAAGTTGGTAGCCAGTACCTGCCCGACACCGACGACCGGCACCGGATCGAGGAAAAGCGGAA
GCGGACCTACGAGACATTCAAGAGCATCATGAAGAAGTCCCCCTTCAGCGGCCCCACCGACCCTAGACCT
CCACCTAGAAGAATCGCCGTGCCCAGCAGATCCAGCGCCAGCGTGCCAAAACCTGCCCCCCAGCCTTACC
CCTTCACCAGCAGCCTGAGCACCATCAACTACGACGAGTTCCCTACCATGGTGTTCCCCAGCGGCCAGAT
CTCTCAGGCCTCTGCTCTGGCTCCAGCCCCTCCTCAGGTGCTGCCTCAGGCTCCTGCTCCTGCACCAGCT
CCAGCCATGGTGTCTGCACTGGCTCAGGCACCAGCACCCGTGCCTGTGCTGGCTCCTGGACCTCCACAGG
CTGTGGCTCCACCAGCCCCTAAACCTACACAGGCCGGCGAGGGCACACTGTCTGAAGCTCTGCTGCAGCT
GCAGTTCGACGACGAGGATCTGGGAGCCCTGCTGGGAAACAGCACCGATCCTGCCGTGTTCACCGACCTG
GCCAGCGTGGACAACAGCGAGTTCCAGCAGCTGCTGAACCAGGGCATCCCTGTGGCCCCTCACACCACCG
AGCCCATGCTGATGGAATACCCCGAGGCCATCACCCGGCTCGTGACAGGCGCTCAGAGGCCTCCTGATCC
AGCTCCTGCCCCTCTGGGAGCACCAGGCCTGCCTAATGGACTGCTGTCTGGCGACGAGGACTTCAGCTCT
ATCGCCGATATGGATTTCTCAGCCTTGCTGGGCTCTGGCAGCGGCAGCCGGGATTCCAGGGAAGGGATGT
TTTTGCCGAAGCCTGAGGCCGGCTCCGCTATTAGTGACGTGTTTGAGGGCCGCGAGGTGTGCCAGCCAAA
ACGAATCCGGCCATTTCATCCTCCAGGAAGTCCATGGGCCAACCGCCCACTCCCCGCCAGCCTCGCACCA
ACACCAACCGGTCCAGTACATGAGCCAGTCGGGTCACTGACCCCGGCACCAGTCCCTCAGCCACTGGATC
CAGCGCCCGCAGTGACTCCCGAGGCCAGTCACCTGTTGGAGGATCCCGATGAAGAGACGAGCCAGGCTGT
CAAAGCCCTTCGGGAGATGGCCGATACTGTGATTCCCCAGAAGGAAGAGGCTGCAATCTGTGGCCAAATG
GACCTTTCCCATCCGCCCCCAAGGGGCCATCTGGATGAGCTGACAACCACACTTGAGTCCATGACCGAGG
ATCTGAACCTGGACTCACCCCTGACCCCGGAATTGAACGAGATTCTGGATACCTTCCTGAACGACGAGTG
CCTCTTGCATGCCATGCATATCAGCACAGGACTGTCCATCTTCGACACATCTCTGTTTTGAccgactcta
gatcataatcagccataccacatttgtagaggttttacttgctttaaaaaacctcccacacctccccctg
aacctgaaacataaaatgaatgcaattgttgttgttaacttgtttattgcagcttataatggttacaaat
aaagcaatagcatcacaaatttcacaaataaagcatttttttcactgcattctagttgtggtttgcccaa
actcatcaatgtatcttaGCGGCTCGAGGGTACCTCTAGAGATCCACTAGTGTCGACGATGTAGGTCACG
GTCTCGAAGCCGCGGTGCGGGTGCCAGGGCGTGCCCTTGGGCTCCCCGGGCGCGTACTCCACCTCACCCA
TCTGGTCCATCATGATGAACGGGTCGAGGTGGCGGTAGTTGATCCCGGCGAACGCGCGGCGCACCGGGAA
GCCCTCGCCCTCGAAACCGCTGGGCGCGGTGGTCACGGTGAGCACGGGACGTGCGACGGCGTCGGCGGGT
GCGGATACGCGGGGCAGCGTCAGCGGGTTCTCGACGGTCACGGCGGGCATGTCGACACTA
SEQ ID NO: 3-pMM7-6-3
CCACNCACGTTTCGTAGTTGCTCTTTCGCTGTCTCCCACCCGCTNTCCGCAACACATTCACCTTTTGTTC
GACGACCNTNGGAGCGACTGTCGTTAGTTCCGCGCGATTCGGTTCGCTCAAATGGTTCCGAGTGGTTCAT
TTCGTCTCAATAGAAATTAGTAATAAATATTTGTATGTACAATTTATTTGCTCCAATATATTTGTATATA
TTTCCCTCACAGCTATATTTATTCTAATTTAATATTATGACTTTTTAAGGTAATTTTTTGTGACCTGTTC
GGAGTGATTAGCGTTACAATTTGAACTGAAAGTGACATCCAGTGTTTGTTCCTTGTGTAGATGCATCTCA
AAAAAATGGTGGGCATAATAGTGTTGTTTATATATATCAAAAATAACAACTATAATAATAAGAATACATT
TAATTTAGAAAATGCTTGGATTTCACTGGAACTAGGCTAGCATAACTTCGTATAATGTATGCTATACGAA
GTTATGCTAGCGGATCCGGGAATTGGGAATTCACGTAAGTACTGTCTGCAGCGTAAGCTTCGTACGTAGC
gaccgtctcaaagtactgcctttctgcgttggaaaacatcgcctttttcgtccaaaaggagtccccaggt
tcgatccgcatggcgttgtgcgtgcgtgcctttcttttcaaatgattacggctattaacttgggggcgtt
aagttggaaacacgtaaattgcagactgcgattagagtgaccatgagtaggagttcaaaatctcctgaca
tcattttcttaaaacctgctttgttttttacatttctatttaatataactcctatttgaataaaaaaaca
aaacaagtttagatgttaagatattaactacatcctttgctccaaagggagaggggaagttatggagtta
attaatttgctgttggaaatcaatatggagtcagaaatataatgatttactaaaccttattgaatcggta
acgatgcgaatttatattaaaatagcttttatgaaacattcaacaaaaatattattaatgttggcccact
ttagcaaccggttaggtctaccggttgggcaagcaaagattcacgccctggttcgagtcccaactagtcc
tgcaaaataccgcagcaagttttagagagaccaagtgccattacctctcccacttcagttatcggttatg
cggcgtttaagtcgacagcttgccgtctctagctccggtgcctatataaagcagcccgctttccacattt
catattcgttttacgtttgtcaagcctcatagccggcagttcgaacgtatacgctctctgagtcagacct
cgaaatcgtagctctacacaattctgtgaattttccttgtcgcgtgtgaaacacttccaatGCaatctta
caaaATGGACAAGAAGTACTCCATTGGGCTCGCTATCGGCACAAACAGCGTCGGCTGGGCCGTCATTACG
GACGAGTACAAGGTGCCGAGCAAAAAATTCAAAGTTCTGGGCAATACCGATCGCCACAGCATAAAGAAGA
ACCTCATTGGCGCCCTCCTGTTCGACTCCGGGGAGACGGCCGAAGCCACGCGGCTCAAAAGAACAGCACG
GCGCAGATATACCCGCAGAAAGAATCGGATCTGCTACCTGCAGGAGATCTTTAGTAATGAGATGGCTAAG
GTGGATGACTCTTTCTTCCATAGGCTGGAGGAGTCCTTTTTGGTGGAGGAGGATAAAAAGCACGAGCGCC
ACCCAATCTTTGGCAATATCGTGGACGAGGTGGCGTACCATGAAAAGTACCCAACCATATATCATCTGAG
GAAGAAGCTTGTAGACAGTACTGATAAGGCTGACTTGCGGTTGATCTATCTCGCGCTGGCGCATATGATC
AAATTTCGGGGACACTTCCTCATCGAGGGGGACCTGAACCCAGACAACAGCGATGTCGATAAACTCTTTA
TCCAACTGGTTCAGACTTACAATCAGCTTTTCGAAGAGAACCCGATCAACGCATCCGGAGTTGACGCCAA
AGCAATCCTGAGCGCTAGGCTGTCCAAATCCCGGCGGCTCGAAAACCTCATCGCACAGCTCCCTGGGGAG
AAGAAGAACGGCCTGTTTGGTAATCTTATCGCCCTGTCACTCGGGCTGACCCCCAACTTTAAATCTAACT
TCGACCTGGCCGAAGATGCCAAGCTTCAACTGAGCAAAGACACCTACGATGATGATCTCGACAATCTGCT
GGCCCAGATCGGCGACCAGTACGCAGACCTTTTTTTGGCGGCAAAGAACCTGTCAGACGCCATTCTGCTG
AGTGATATTCTGCGAGTGAACACGGAGATCACCAAAGCTCCGCTGAGCGCTAGTATGATCAAGCGCTATG
ATGAGCACCACCAAGACTTGACTTTGCTGAAGGCCCTTGTCAGACAGCAACTGCCTGAGAAGTACAAGGA
AATTTTCTTCGATCAGTCTAAAAATGGCTACGCCGGATACATTGACGGCGGAGCAAGCCAGGAGGAATTT
TACAAATTTATTAAGCCCATCTTGGAAAAAATGGACGGCACCGAGGAGCTGCTGGTAAAGCTTAACAGAG
AAGATCTGTTGCGCAAACAGCGCACTTTCGACAATGGAAGCATCCCCCACCAGATTCACCTGGGCGAACT
GCACGCTATCCTCAGGCGGCAAGAGGATTTCTACCCCTTTTTGAAAGATAACAGGGAAAAGATTGAGAAA
ATCCTCACATTTCGGATACCCTACTATGTAGGCCCCCTCGCCCGGGGAAATTCCAGATTCGCGTGGATGA
CTCGCAAATCAGAAGAGACCATCACTCCCTGGAACTTCGAGGAAGTCGTGGATAAGGGGGCCTCTGCCCA
GTCCTTCATCGAAAGGATGACTAACTTTGATAAAAATCTGCCTAACGAAAAGGTGCTTCCTAAACACTCT
CTGCTGTACGAGTACTTCACAGTTTATAACGAGCTCACCAAGGTCAAATACGTCACAGAAGGGATGAGAA
AGCCAGCATTCCTGTCTGGAGAGCAGAAGAAAGCTATCGTGGACCTCCTCTTCAAGACGAACCGGAAAGT
TACCGTGAAACAGCTCAAAGAAGACTATTTCAAAAAGATTGAATGTTTCGACTCTGTTGAAATCAGCGGA
GTGGAGGATCGCTTCAACGCATCCCTGGGAACGTATCACGATCTCCTGAAAATCATTAAAGACAAGGACT
TCCTGGACAATGAGGAGAACGAGGACATTCTTGAGGACATTGTCCTCACCCTTACGTTGTTTGAAGATAG
GGAGATGATTGAAGAACGCTTGAAAACTTACGCTCATCTCTTCGACGACAAAGTCATGAAACAGCTCAAG
AGGCGCCGATATACAGGATGGGGGCGGCTGTCAAGAAAACTGATCAATGGGATCCGAGACAAGCAGAGTG
GAAAGACAATCCTGGATTTTCTTAAGTCCGATGGATTTGCCAACCGGAACTTCATGCAGTTGATCCATGA
TGACTCTCTCACCTTTAAGGAGGACATCCAGAAAGCACAAGTTTCTGGCCAGGGGGACAGTCTTCACGAG
CACATCGCTAATCTTGCAGGTAGCCCAGCTATCAAAAAGGGAATACTGCAGACCGTTAAGGTCGTGGATG
AACTCGTCAAAGTAATGGGAAGGCATAAGCCCGAGAATATCGTTATCGAGATGGCCCGAGAGAACCAAAC
TACCCAGAAGGGACAGAAGAACAGTAGGGAAAGGATGAAGAGGATTGAAGAGGGTATAAAAGAACTGGGG
TCCCAAATCCTTAAGGAACACCCAGTTGAAAACACCCAGCTTCAGAATGAGAAGCTCTACCTGTACTACC
TGCAGAACGGCAGGGACATGTACGTGGATCAGGAACTGGACATCAATCGGCTCTCCGACTACGACGTGGC
TGCTATCGTGCCCCAGTCTTTTCTCAAAGATGATTCTATTGATAATAAAGTGTTGACAAGATCCGATAAA
GCTAGAGGGAAGAGTGATAACGTCCCCTCAGAAGAAGTTGTCAAGAAAATGAAAAATTATTGGCGGCAGC
TGCTGAACGCCAAACTGATCACACAACGGAAGTTCGATAATCTGACTAAGGCTGAACGAGGTGGCCTGTC
TGAGTTGGATAAAGCCGGCTTCATCAAAAGGCAGCTTGTTGAGACACGCCAGATCACCAAGCACGTGGCC
CAAATTCTCGATTCACGCATGAACACCAAGTACGATGAAAATGACAAACTGATTCGAGAGGTGAAAGTTA
TTACTCTGAAGTCTAAGCTGGTCTCAGATTTCAGAAAGGACTTTCAGTTTTATAAGGTGAGAGAGATCAA
CAATTACCACCATGCGCATGATGCCTACCTGAATGCAGTGGTAGGCACTGCACTTATCAAAAAATATCCC
AAGCTTGAATCTGAATTTGTTTACGGAGACTATAAAGTGTACGATGTTAGGAAAATGATCGCAAAGTCTG
AGCAGGAAATAGGCAAGGCCACCGCTAAGTACTTCTTTTACAGCAATATTATGAATTTTTTCAAGACCGA
GATTACACTGGCCAATGGAGAGATTCGGAAGCGACCACTTATCGAAACAAACGGAGAAACAGGAGAAATC
GTGTGGGACAAGGGTAGGGATTTCGCGACAGTCCGGAAGGTCCTGTCCATGCCGCAGGTGAACATCGTTA
AAAAGACCGAAGTACAGACCGGAGGCTTCTCCAAGGAAAGTATCCTCCCGAAAAGGAACAGCGACAAGCT
GATCGCACGCAAAAAAGATTGGGACCCCAAGAAATACGGCGGATTCGATTCTCCTACAGTCGCTTACAGT
GTACTGGTTGTGGCCAAAGTGGAGAAAGGGAAGTCTAAAAAACTCAAAAGCGTCAAGGAACTGCTGGGCA
TCACAATCATGGAGCGATCAAGCTTCGAAAAAAACCCCATCGACTTTCTCGAGGCGAAAGGATATAAAGA
GGTCAAAAAAGACCTCATCATTAAGCTTCCCAAGTACTCTCTCTTTGAGCTTGAAAACGGCCGGAAACGA
ATGCTCGCTAGTGCGGGCGAGCTGCAGAAAGGTAACGAGCTGGCACTGCCCTCTAAATACGTTAATTTCT
TGTATCTGGCCAGCCACTATGAAAAGCTCAAAGGGTCTCCCGAAGATAATGAGCAGAAGCAGCTGTTCGT
GGAACAACACAAACACTACCTTGATGAGATCATCGAGCAAATAAGCGAATTCTCCAAAAGAGTGATCCTC
GCCGACGCTAACCTCGATAAGGTGCTTTCTGCTTACAATAAGCACAGGGATAAGCCCATCAGGGAGCAGG
CAGAAAACATTATCCACTTGTTTACTCTGACCAACTTGGGCGCGCCTGCAGCCTTCAAGTACTTCGACAC
CACCATAGACAGAAAGCGGTACACCTCTACAAAGGAGGTCCTGGACGCCACACTGATTCATCAGTCAATT
ACGGGGCTCTATGAAACAAGAATCGACCTCTCTCAGCTCGGTGGAGACAGCAGGGCTGACCCCAAGAAGA
AGAGGAAGGTGGAGGCCAGCGGTTCCGGACGGGCTGACGCATTGGACGATTTTGATCTGGATATGCTGGG
AAGTGACGCCCTCGATGATTTTGACCTTGACATGCTTGGTTCGGATGCCCTTGATGACTTTGACCTCGAC
ATGCTCGGCAGTGACGCCCTTGATGATTTCGACCTGGACATGCTGATTAACTCTAGAAGTTCCGGATCTC
CGAAAAAGAAACGCAAAGTTGGTAGCCAGTACCTGCCCGACACCGACGACCGGCACCGGATCGAGGAAAA
GCGGAAGCGGACCTACGAGACATTCAAGAGCATCATGAAGAAGTCCCCCTTCAGCGGCCCCACCGACCCT
AGACCTCCACCTAGAAGAATCGCCGTGCCCAGCAGATCCAGCGCCAGCGTGCCAAAACCTGCCCCCCAGC
CTTACCCCTTCACCAGCAGCCTGAGCACCATCAACTACGACGAGTTCCCTACCATGGTGTTCCCCAGCGG
CCAGATCTCTCAGGCCTCTGCTCTGGCTCCAGCCCCTCCTCAGGTGCTGCCTCAGGCTCCTGCTCCTGCA
CCAGCTCCAGCCATGGTGTCTGCACTGGCTCAGGCACCAGCACCCGTGCCTGTGCTGGCTCCTGGACCTC
CACAGGCTGTGGCTCCACCAGCCCCTAAACCTACACAGGCCGGCGAGGGCACACTGTCTGAAGCTCTGCT
GCAGCTGCAGTTCGACGACGAGGATCTGGGAGCCCTGCTGGGAAACAGCACCGATCCTGCCGTGTTCACC
GACCTGGCCAGCGTGGACAACAGCGAGTTCCAGCAGCTGCTGAACCAGGGCATCCCTGTGGCCCCTCACA
CCACCGAGCCCATGCTGATGGAATACCCCGAGGCCATCACCCGGCTCGTGACAGGCGCTCAGAGGCCTCC
TGATCCAGCTCCTGCCCCTCTGGGAGCACCAGGCCTGCCTAATGGACTGCTGTCTGGCGACGAGGACTTC
AGCTCTATCGCCGATATGGATTTCTCAGCCTTGCTGGGCTCTGGCAGCGGCAGCCGGGATTCCAGGGAAG
GGATGTTTTTGCCGAAGCCTGAGGCCGGCTCCGCTATTAGTGACGTGTTTGAGGGCCGCGAGGTGTGCCA
GCCAAAACGAATCCGGCCATTTCATCCTCCAGGAAGTCCATGGGCCAACCGCCCACTCCCCGCCAGCCTC
GCACCAACACCAACCGGTCCAGTACATGAGCCAGTCGGGTCACTGACCCCGGCACCAGTCCCTCAGCCAC
TGGATCCAGCGCCCGCAGTGACTCCCGAGGCCAGTCACCTGTTGGAGGATCCCGATGAAGAGACGAGCCA
GGCTGTCAAAGCCCTTCGGGAGATGGCCGATACTGTGATTCCCCAGAAGGAAGAGGCTGCAATCTGTGGC
CAAATGGACCTTTCCCATCCGCCCCCAAGGGGCCATCTGGATGAGCTGACAACCACACTTGAGTCCATGA
CCGAGGATCTGAACCTGGACTCACCCCTGACCCCGGAATTGAACGAGATTCTGGATACCTTCCTGAACGA
CGAGTGCCTCTTGCATGCCATGCATATCAGCACAGGACTGTCCATCTTCGACACATCTCTGTTTTGAccg
actctagatcataatcagccataccacatttgtagaggttttacttgctttaaaaaacctcccacacctc
cccctgaacctgaaacataaaatgaatgcaattgttgttgttaacttgtttattgcagcttataatggtt
acaaataaagcaatagcatcacaaatttcacaaataaagcatttttttcactgcattctagttgtggttt
gcccaaactcatcaatgtatcttaGCGGCTCGAGGGTACCTCTAGAGATCCACTAGTGTCGACGATGTAG
GTCACGGTCTCGAAGCCGCGGTGCGGGTGCCAGGGCGTGCCCTTGGGCTCCCCGGGCGCGTACTCCACCT
CACCCATCTGGTCCATCATGATGAACGGGTCGAGGTGGCGGTAGTTGATCCCGGCGAACGCGCGGCGCAC
CGGGAAGCCCTCGCCCTCGAAACCGCTGGGCGCGGTGGTCACGGTGAGCACGGGACGTGCGACGGCGTCG
GCGGGTGCGGATACGCGGGGCAGCGTCAGCGGGTTCTCGACGGTCACGGCGGGCATGTCGACACTA
SEQ ID NO: 4-pMM7-6-4
CCACNCACGTTTCGTAGTTGCTCTTTCGCTGTCTCCCACCCGCTNTCCGCAACACATTCACCTTTTGTTC
GACGACCNTNGGAGCGACTGTCGTTAGTTCCGCGCGATTCGGTTCGCTCAAATGGTTCCGAGTGGTTCAT
TTCGTCTCAATAGAAATTAGTAATAAATATTTGTATGTACAATTTATTTGCTCCAATATATTTGTATATA
TTTCCCTCACAGCTATATTTATTCTAATTTAATATTATGACTTTTTAAGGTAATTTTTTGTGACCTGTTC
GGAGTGATTAGCGTTACAATTTGAACTGAAAGTGACATCCAGTGTTTGTTCCTTGTGTAGATGCATCTCA
AAAAAATGGTGGGCATAATAGTGTTGTTTATATATATCAAAAATAACAACTATAATAATAAGAATACATT
TAATTTAGAAAATGCTTGGATTTCACTGGAACTAGGCTAGCATAACTTCGTATAATGTATGCTATACGAA
GTTATGCTAGCGGATCCGGGAATTGGGAATTCACGTAAGTACTGTCTGCAGCGTAAGCTTCGTACGTAGC
cagctccttgttggttgaccaaatcgtagaccttcaataaatttccaaggacacgcaccactcgtttgga
aaatatttggttctcttcaaggttgaaactctcgggggttttggacgtcgatcggttttggtttgtttag
aattgttgtggcgttgttttacgaatagatattactttgatggattagccatatcgcattgaaggtcgcc
tcttggttagcctcgaatttgttacgacctgttttgtgttggctaaccaaaatacaaactccgcacatac
agtgcgagcaacaattatgcagaaaaaacgcatgaatagcttatggaatttacactttgacggatgaaga
aatatacttcctttttccatattcgatattatcgtagagatagaataagatatattgttctaaattcctt
ttatgtcctactatttctttgatttgattaaaaatgtgtcttcccaagaagaacttaattgcctcagata
attgcttatcgtaaagaaattaccaccactctcgctatctgccattagttaatgaatagacccaccagac
tttagtagctctgccgatttgggttatttttacaacctcggtgggcgagcgggatggaagacgaggagag
gtgatcactacctacagcagcgagaggtttggattttatgatatatttacattccagccttctgttctta
ctcactcgccgtgaatgtctgggagtgcgtgtgtgtctcatcatttggattttccgcggcaataaaatta
ataagaacgcgctaatttttcaggccccggggcctaagcaaataaacatacactatttcctgcaactcct
ccacccttttcccctaactcttttccagcgcccagactgtgctaatatttgccaagggatattattgggc
ctaaaccgaaaacggaactctttccgttcgccattttgttggccagcaaagcgcttttcctgttgttgtt
taccgatgaattgaaaaataaatgaatatatttatttggaacatttatgttttgtcctacactataatta
atttaaaatcactatcagttctggcaggctctaaacagcgaattaatgtttaattcattgaaaatggctg
aaaaaaaagtgttctataggtggggaagatagcccctaaaggtggggtgggataccagctcttcttgggc
tgcacaaactgtccaattagtggaagcggccaagcaatggatgaggaaaaggtaagacataaactcggtt
cggaatgccaaagtgtgtggtaacaatcccctgagagtgagggagctggctgcatccaagtgcagtatat
aagactactccgaaatttactccgaaaagcagcagaaaacttgttctgacacggcaaatgtatggaaagg
tttaaggaaacaggcatattaaagaaacttcttgttaattgtttctaaatatttatatttatagagtagc
taaatttagttgctatcgatttaagaatactttcatagccaaaagctagaagttaaaagtagtaatacca
ctttttcacccataagctaaagataaaacccaaattcaacagtcgaaaataatagttcaaagcctttatt
agccgaacagtaagcgtaacaaaatcaccataaaaaaccaatcccataaatatcttacagaaataggcga
aaatattgcgacaaatatgtataattaaatgtagtcaaagctatgacgaaattcatgaggttgcgcaaat
aatcgggcaatacaatcgattacaccgaaaatgcaccgagtttttccatttccgccatttcttattgggc
catgctggctatataccgcacacacacacacgcacgcacacttcaaagcgcaacacacaagaaacgttta
cgaagagacagggagaacgaacgatcaccgcgccatatagcggtgctcttctggcgcacgcagctgcaat
gcaggagtcagggtatagctccaccccactcgcacacacacaccatcgggcggtcgtgtatgcgatccga
agacgaagaccgacgatgcgatcggatcggggatctcgggtcgctgctgacaaacgcagagtcggacgaa
agaacgcaccgtgtgtttcagttaagcgttggcactgaaccgggcaacaatcttcactcctccgctcgaa
acgccgcgatcgaaccgatctataactagccatctataactagagcgagccgagtgtattctatcgaaac
agccaaatttacgatacaatatatatttgtatatgcgtggaaaacttacaagttcttgttgtgtcccatg
attgccgtgtgatccagcggaattaatcgcacaaatatgagcagcaatatcggcatacgcatgctaatga
tgattatgcctcatttatagtgcgctaattgaacgcgaaattgctcgatacattcaatataaccaaacca
ttcgcaaacaaacaacaactcgaagggaagtatctatcataccccgtgtgtcagtgtgagagtgtgtgtg
ccgtcgaacagataaacccgatcagcGCaatcttacaaaATGGACAAGAAGTACTCCATTGGGCTCGCTA
TCGGCACAAACAGCGTCGGCTGGGCCGTCATTACGGACGAGTACAAGGTGCCGAGCAAAAAATTCAAAGT
TCTGGGCAATACCGATCGCCACAGCATAAAGAAGAACCTCATTGGCGCCCTCCTGTTCGACTCCGGGGAG
ACGGCCGAAGCCACGCGGCTCAAAAGAACAGCACGGCGCAGATATACCCGCAGAAAGAATCGGATCTGCT
ACCTGCAGGAGATCTTTAGTAATGAGATGGCTAAGGTGGATGACTCTTTCTTCCATAGGCTGGAGGAGTC
CTTTTTGGTGGAGGAGGATAAAAAGCACGAGCGCCACCCAATCTTTGGCAATATCGTGGACGAGGTGGCG
TACCATGAAAAGTACCCAACCATATATCATCTGAGGAAGAAGCTTGTAGACAGTACTGATAAGGCTGACT
TGCGGTTGATCTATCTCGCGCTGGCGCATATGATCAAATTTCGGGGACACTTCCTCATCGAGGGGGACCT
GAACCCAGACAACAGCGATGTCGATAAACTCTTTATCCAACTGGTTCAGACTTACAATCAGCTTTTCGAA
GAGAACCCGATCAACGCATCCGGAGTTGACGCCAAAGCAATCCTGAGCGCTAGGCTGTCCAAATCCCGGC
GGCTCGAAAACCTCATCGCACAGCTCCCTGGGGAGAAGAAGAACGGCCTGTTTGGTAATCTTATCGCCCT
GTCACTCGGGCTGACCCCCAACTTTAAATCTAACTTCGACCTGGCCGAAGATGCCAAGCTTCAACTGAGC
AAAGACACCTACGATGATGATCTCGACAATCTGCTGGCCCAGATCGGCGACCAGTACGCAGACCTTTTTT
TGGCGGCAAAGAACCTGTCAGACGCCATTCTGCTGAGTGATATTCTGCGAGTGAACACGGAGATCACCAA
AGCTCCGCTGAGCGCTAGTATGATCAAGCGCTATGATGAGCACCACCAAGACTTGACTTTGCTGAAGGCC
CTTGTCAGACAGCAACTGCCTGAGAAGTACAAGGAAATTTTCTTCGATCAGTCTAAAAATGGCTACGCCG
GATACATTGACGGCGGAGCAAGCCAGGAGGAATTTTACAAATTTATTAAGCCCATCTTGGAAAAAATGGA
CGGCACCGAGGAGCTGCTGGTAAAGCTTAACAGAGAAGATCTGTTGCGCAAACAGCGCACTTTCGACAAT
GGAAGCATCCCCCACCAGATTCACCTGGGCGAACTGCACGCTATCCTCAGGCGGCAAGAGGATTTCTACC
CCTTTTTGAAAGATAACAGGGAAAAGATTGAGAAAATCCTCACATTTCGGATACCCTACTATGTAGGCCC
CCTCGCCCGGGGAAATTCCAGATTCGCGTGGATGACTCGCAAATCAGAAGAGACCATCACTCCCTGGAAC
TTCGAGGAAGTCGTGGATAAGGGGGCCTCTGCCCAGTCCTTCATCGAAAGGATGACTAACTTTGATAAAA
ATCTGCCTAACGAAAAGGTGCTTCCTAAACACTCTCTGCTGTACGAGTACTTCACAGTTTATAACGAGCT
CACCAAGGTCAAATACGTCACAGAAGGGATGAGAAAGCCAGCATTCCTGTCTGGAGAGCAGAAGAAAGCT
ATCGTGGACCTCCTCTTCAAGACGAACCGGAAAGTTACCGTGAAACAGCTCAAAGAAGACTATTTCAAAA
AGATTGAATGTTTCGACTCTGTTGAAATCAGCGGAGTGGAGGATCGCTTCAACGCATCCCTGGGAACGTA
TCACGATCTCCTGAAAATCATTAAAGACAAGGACTTCCTGGACAATGAGGAGAACGAGGACATTCTTGAG
GACATTGTCCTCACCCTTACGTTGTTTGAAGATAGGGAGATGATTGAAGAACGCTTGAAAACTTACGCTC
ATCTCTTCGACGACAAAGTCATGAAACAGCTCAAGAGGCGCCGATATACAGGATGGGGGCGGCTGTCAAG
AAAACTGATCAATGGGATCCGAGACAAGCAGAGTGGAAAGACAATCCTGGATTTTCTTAAGTCCGATGGA
TTTGCCAACCGGAACTTCATGCAGTTGATCCATGATGACTCTCTCACCTTTAAGGAGGACATCCAGAAAG
CACAAGTTTCTGGCCAGGGGGACAGTCTTCACGAGCACATCGCTAATCTTGCAGGTAGCCCAGCTATCAA
AAAGGGAATACTGCAGACCGTTAAGGTCGTGGATGAACTCGTCAAAGTAATGGGAAGGCATAAGCCCGAG
AATATCGTTATCGAGATGGCCCGAGAGAACCAAACTACCCAGAAGGGACAGAAGAACAGTAGGGAAAGGA
TGAAGAGGATTGAAGAGGGTATAAAAGAACTGGGGTCCCAAATCCTTAAGGAACACCCAGTTGAAAACAC
CCAGCTTCAGAATGAGAAGCTCTACCTGTACTACCTGCAGAACGGCAGGGACATGTACGTGGATCAGGAA
CTGGACATCAATCGGCTCTCCGACTACGACGTGGCTGCTATCGTGCCCCAGTCTTTTCTCAAAGATGATT
CTATTGATAATAAAGTGTTGACAAGATCCGATAAAGCTAGAGGGAAGAGTGATAACGTCCCCTCAGAAGA
AGTTGTCAAGAAAATGAAAAATTATTGGCGGCAGCTGCTGAACGCCAAACTGATCACACAACGGAAGTTC
GATAATCTGACTAAGGCTGAACGAGGTGGCCTGTCTGAGTTGGATAAAGCCGGCTTCATCAAAAGGCAGC
TTGTTGAGACACGCCAGATCACCAAGCACGTGGCCCAAATTCTCGATTCACGCATGAACACCAAGTACGA
TGAAAATGACAAACTGATTCGAGAGGTGAAAGTTATTACTCTGAAGTCTAAGCTGGTCTCAGATTTCAGA
AAGGACTTTCAGTTTTATAAGGTGAGAGAGATCAACAATTACCACCATGCGCATGATGCCTACCTGAATG
CAGTGGTAGGCACTGCACTTATCAAAAAATATCCCAAGCTTGAATCTGAATTTGTTTACGGAGACTATAA
AGTGTACGATGTTAGGAAAATGATCGCAAAGTCTGAGCAGGAAATAGGCAAGGCCACCGCTAAGTACTTC
TTTTACAGCAATATTATGAATTTTTTCAAGACCGAGATTACACTGGCCAATGGAGAGATTCGGAAGCGAC
CACTTATCGAAACAAACGGAGAAACAGGAGAAATCGTGTGGGACAAGGGTAGGGATTTCGCGACAGTCCG
GAAGGTCCTGTCCATGCCGCAGGTGAACATCGTTAAAAAGACCGAAGTACAGACCGGAGGCTTCTCCAAG
GAAAGTATCCTCCCGAAAAGGAACAGCGACAAGCTGATCGCACGCAAAAAAGATTGGGACCCCAAGAAAT
ACGGCGGATTCGATTCTCCTACAGTCGCTTACAGTGTACTGGTTGTGGCCAAAGTGGAGAAAGGGAAGTC
TAAAAAACTCAAAAGCGTCAAGGAACTGCTGGGCATCACAATCATGGAGCGATCAAGCTTCGAAAAAAAC
CCCATCGACTTTCTCGAGGCGAAAGGATATAAAGAGGTCAAAAAAGACCTCATCATTAAGCTTCCCAAGT
ACTCTCTCTTTGAGCTTGAAAACGGCCGGAAACGAATGCTCGCTAGTGCGGGCGAGCTGCAGAAAGGTAA
CGAGCTGGCACTGCCCTCTAAATACGTTAATTTCTTGTATCTGGCCAGCCACTATGAAAAGCTCAAAGGG
TCTCCCGAAGATAATGAGCAGAAGCAGCTGTTCGTGGAACAACACAAACACTACCTTGATGAGATCATCG
AGCAAATAAGCGAATTCTCCAAAAGAGTGATCCTCGCCGACGCTAACCTCGATAAGGTGCTTTCTGCTTA
CAATAAGCACAGGGATAAGCCCATCAGGGAGCAGGCAGAAAACATTATCCACTTGTTTACTCTGACCAAC
TTGGGCGCGCCTGCAGCCTTCAAGTACTTCGACACCACCATAGACAGAAAGCGGTACACCTCTACAAAGG
AGGTCCTGGACGCCACACTGATTCATCAGTCAATTACGGGGCTCTATGAAACAAGAATCGACCTCTCTCA
GCTCGGTGGAGACAGCAGGGCTGACCCCAAGAAGAAGAGGAAGGTGGAGGCCAGCGGTTCCGGACGGGCT
GACGCATTGGACGATTTTGATCTGGATATGCTGGGAAGTGACGCCCTCGATGATTTTGACCTTGACATGC
TTGGTTCGGATGCCCTTGATGACTTTGACCTCGACATGCTCGGCAGTGACGCCCTTGATGATTTCGACCT
GGACATGCTGATTAACTCTAGAAGTTCCGGATCTCCGAAAAAGAAACGCAAAGTTGGTAGCCAGTACCTG
CCCGACACCGACGACCGGCACCGGATCGAGGAAAAGCGGAAGCGGACCTACGAGACATTCAAGAGCATCA
TGAAGAAGTCCCCCTTCAGCGGCCCCACCGACCCTAGACCTCCACCTAGAAGAATCGCCGTGCCCAGCAG
ATCCAGCGCCAGCGTGCCAAAACCTGCCCCCCAGCCTTACCCCTTCACCAGCAGCCTGAGCACCATCAAC
TACGACGAGTTCCCTACCATGGTGTTCCCCAGCGGCCAGATCTCTCAGGCCTCTGCTCTGGCTCCAGCCC
CTCCTCAGGTGCTGCCTCAGGCTCCTGCTCCTGCACCAGCTCCAGCCATGGTGTCTGCACTGGCTCAGGC
ACCAGCACCCGTGCCTGTGCTGGCTCCTGGACCTCCACAGGCTGTGGCTCCACCAGCCCCTAAACCTACA
CAGGCCGGCGAGGGCACACTGTCTGAAGCTCTGCTGCAGCTGCAGTTCGACGACGAGGATCTGGGAGCCC
TGCTGGGAAACAGCACCGATCCTGCCGTGTTCACCGACCTGGCCAGCGTGGACAACAGCGAGTTCCAGCA
GCTGCTGAACCAGGGCATCCCTGTGGCCCCTCACACCACCGAGCCCATGCTGATGGAATACCCCGAGGCC
ATCACCCGGCTCGTGACAGGCGCTCAGAGGCCTCCTGATCCAGCTCCTGCCCCTCTGGGAGCACCAGGCC
TGCCTAATGGACTGCTGTCTGGCGACGAGGACTTCAGCTCTATCGCCGATATGGATTTCTCAGCCTTGCT
GGGCTCTGGCAGCGGCAGCCGGGATTCCAGGGAAGGGATGTTTTTGCCGAAGCCTGAGGCCGGCTCCGCT
ATTAGTGACGTGTTTGAGGGCCGCGAGGTGTGCCAGCCAAAACGAATCCGGCCATTTCATCCTCCAGGAA
GTCCATGGGCCAACCGCCCACTCCCCGCCAGCCTCGCACCAACACCAACCGGTCCAGTACATGAGCCAGT
CGGGTCACTGACCCCGGCACCAGTCCCTCAGCCACTGGATCCAGCGCCCGCAGTGACTCCCGAGGCCAGT
CACCTGTTGGAGGATCCCGATGAAGAGACGAGCCAGGCTGTCAAAGCCCTTCGGGAGATGGCCGATACTG
TGATTCCCCAGAAGGAAGAGGCTGCAATCTGTGGCCAAATGGACCTTTCCCATCCGCCCCCAAGGGGCCA
TCTGGATGAGCTGACAACCACACTTGAGTCCATGACCGAGGATCTGAACCTGGACTCACCCCTGACCCCG
GAATTGAACGAGATTCTGGATACCTTCCTGAACGACGAGTGCCTCTTGCATGCCATGCATATCAGCACAG
GACTGTCCATCTTCGACACATCTCTGTTTTGAccgactctagatcataatcagccataccacatttgtag
aggttttacttgctttaaaaaacctcccacacctccccctgaacctgaaacataaaatgaatgcaattgt
tgttgttaacttgtttattgcagcttataatggttacaaataaagcaatagcatcacaaatttcacaaat
aaagcatttttttcactgcattctagttgtggtttgcccaaactcatcaatgtatcttaGCGGCTCGAGG
GTACCTCTAGAGATCCACTAGTGTCGACGATGTAGGTCACGGTCTCGAAGCCGCGGTGCGGGTGCCAGGG
CGTGCCCTTGGGCTCCCCGGGCGCGTACTCCACCTCACCCATCTGGTCCATCATGATGAACGGGTCGAGG
TGGCGGTAGTTGATCCCGGCGAACGCGCGGCGCACCGGGAAGCCCTCGCCCTCGAAACCGCTGGGCGCGG
TGGTCACGGTGAGCACGGGACGTGCGACGGCGTCGGCGGGTGCGGATACGCGGGGCAGCGTCAGCGGGTT
CTCGACGGTCACGGCGGGCATGTCGACACTA
SEQ ID NO: 5-pMM7-6-5
CCACNCACGTTTCGTAGTTGCTCTTTCGCTGTCTCCCACCCGCTNTCCGCAACACATTCACCTTTTGTTC
GACGACCNTNGGAGCGACTGTCGTTAGTTCCGCGCGATTCGGTTCGCTCAAATGGTTCCGAGTGGTTCAT
TTCGTCTCAATAGAAATTAGTAATAAATATTTGTATGTACAATTTATTTGCTCCAATATATTTGTATATA
TTTCCCTCACAGCTATATTTATTCTAATTTAATATTATGACTTTTTAAGGTAATTTTTTGTGACCTGTTC
GGAGTGATTAGCGTTACAATTTGAACTGAAAGTGACATCCAGTGTTTGTTCCTTGTGTAGATGCATCTCA
AAAAAATGGTGGGCATAATAGTGTTGTTTATATATATCAAAAATAACAACTATAATAATAAGAATACATT
TAATTTAGAAAATGCTTGGATTTCACTGGAACTAGGCTAGCATAACTTCGTATAATGTATGCTATACGAA
GTTATGCTAGCGGATCCGGGAATTGGGAATTCACGTAAGTACTGTCTGCAGCGTAAGCTTCGTACGTAGC
actctaaaacgtaaagaaaccacagaacccatacgagagaaagcttgtaattcaattgctgcggtccttt
ggttcattgtgctttgtgaattaaagaattaacgatgttgtggtcggctaagtgaaaaaaaaaacagttc
ttgtcgtatttgtttatagaaagtggataattgccaacaggatagatagtggagctcaatcgctggggtt
ccccgataagaaaccgcccataatggaagctcttgtgtgtgcaaatacccttgtgcggcaaaacttcagg
aatttttcactagttatgcttagatctaaccattgattaacttcacaacaataaagaatgtttcataggc
tctaaatcgagattttgtgaggcttctaatgattgggcattcagcattttttcaagaattttgtaaccga
ctcaaaaaatctttagaatggttggttattcggatcgcatatacttagcttgtttgtcttatttttattt
ggatgagcgccaaaattttgctgcgtcagtctggaaaaaattgaatcaaatgtgtatagttttatagaag
ttgggaagcggaatttatttatttatttaaatatttataattaaaaaaatgaaaatagtcacgttgttta
actagtcagtattcgaaccaacaaatgtaaaatgtatactggtttgtgtctaagctaagcttgtcatatt
aacggagctgccagatgttaggaagtggggatgccatacattattctaaatttgcgcgcaattttagaag
cttatcgtcgtcagaattacaaaaacaaattgaatatgaaaatgggttattgctacttcattattattgt
cacgatatatgataatttatacaaaatgtgataaatcccaaattgttaaataatgctttggcttgcttta
tacaaaaccactagataattaaaatataggtggcctaaattgttgcatgttgttttataattaatcagca
atttgatttggttgtgatcgaccaaatcagtgtgtataattgtagttaaaatgtaaagttcgtaatggat
tattgaatcgcatttcaaatttctttaaatgcgcccgggtcaatgaccttttgaggtgaccataaattga
aacttatttgtgcgacggcaaccctgttctgggactcgacatgatatcgatacgttaacaacaaagagtc
tggacgccatcattcttcctctttctcctgaattcgcagacagcgtggcgtcaggcatttcaaacggcaa
aaagaacctggcgataaggaaagatttaaaaggcaaaaatcgagtgatttgtgtgatttaacttaagGCa
atcttacaaaATGGACAAGAAGTACTCCATTGGGCTCGCTATCGGCACAAACAGCGTCGGCTGGGCCGTC
ATTACGGACGAGTACAAGGTGCCGAGCAAAAAATTCAAAGTTCTGGGCAATACCGATCGCCACAGCATAA
AGAAGAACCTCATTGGCGCCCTCCTGTTCGACTCCGGGGAGACGGCCGAAGCCACGCGGCTCAAAAGAAC
AGCACGGCGCAGATATACCCGCAGAAAGAATCGGATCTGCTACCTGCAGGAGATCTTTAGTAATGAGATG
GCTAAGGTGGATGACTCTTTCTTCCATAGGCTGGAGGAGTCCTTTTTGGTGGAGGAGGATAAAAAGCACG
AGCGCCACCCAATCTTTGGCAATATCGTGGACGAGGTGGCGTACCATGAAAAGTACCCAACCATATATCA
TCTGAGGAAGAAGCTTGTAGACAGTACTGATAAGGCTGACTTGCGGTTGATCTATCTCGCGCTGGCGCAT
ATGATCAAATTTCGGGGACACTTCCTCATCGAGGGGGACCTGAACCCAGACAACAGCGATGTCGATAAAC
TCTTTATCCAACTGGTTCAGACTTACAATCAGCTTTTCGAAGAGAACCCGATCAACGCATCCGGAGTTGA
CGCCAAAGCAATCCTGAGCGCTAGGCTGTCCAAATCCCGGCGGCTCGAAAACCTCATCGCACAGCTCCCT
GGGGAGAAGAAGAACGGCCTGTTTGGTAATCTTATCGCCCTGTCACTCGGGCTGACCCCCAACTTTAAAT
CTAACTTCGACCTGGCCGAAGATGCCAAGCTTCAACTGAGCAAAGACACCTACGATGATGATCTCGACAA
TCTGCTGGCCCAGATCGGCGACCAGTACGCAGACCTTTTTTTGGCGGCAAAGAACCTGTCAGACGCCATT
CTGCTGAGTGATATTCTGCGAGTGAACACGGAGATCACCAAAGCTCCGCTGAGCGCTAGTATGATCAAGC
GCTATGATGAGCACCACCAAGACTTGACTTTGCTGAAGGCCCTTGTCAGACAGCAACTGCCTGAGAAGTA
CAAGGAAATTTTCTTCGATCAGTCTAAAAATGGCTACGCCGGATACATTGACGGCGGAGCAAGCCAGGAG
GAATTTTACAAATTTATTAAGCCCATCTTGGAAAAAATGGACGGCACCGAGGAGCTGCTGGTAAAGCTTA
ACAGAGAAGATCTGTTGCGCAAACAGCGCACTTTCGACAATGGAAGCATCCCCCACCAGATTCACCTGGG
CGAACTGCACGCTATCCTCAGGCGGCAAGAGGATTTCTACCCCTTTTTGAAAGATAACAGGGAAAAGATT
GAGAAAATCCTCACATTTCGGATACCCTACTATGTAGGCCCCCTCGCCCGGGGAAATTCCAGATTCGCGT
GGATGACTCGCAAATCAGAAGAGACCATCACTCCCTGGAACTTCGAGGAAGTCGTGGATAAGGGGGCCTC
TGCCCAGTCCTTCATCGAAAGGATGACTAACTTTGATAAAAATCTGCCTAACGAAAAGGTGCTTCCTAAA
CACTCTCTGCTGTACGAGTACTTCACAGTTTATAACGAGCTCACCAAGGTCAAATACGTCACAGAAGGGA
TGAGAAAGCCAGCATTCCTGTCTGGAGAGCAGAAGAAAGCTATCGTGGACCTCCTCTTCAAGACGAACCG
GAAAGTTACCGTGAAACAGCTCAAAGAAGACTATTTCAAAAAGATTGAATGTTTCGACTCTGTTGAAATC
AGCGGAGTGGAGGATCGCTTCAACGCATCCCTGGGAACGTATCACGATCTCCTGAAAATCATTAAAGACA
AGGACTTCCTGGACAATGAGGAGAACGAGGACATTCTTGAGGACATTGTCCTCACCCTTACGTTGTTTGA
AGATAGGGAGATGATTGAAGAACGCTTGAAAACTTACGCTCATCTCTTCGACGACAAAGTCATGAAACAG
CTCAAGAGGCGCCGATATACAGGATGGGGGCGGCTGTCAAGAAAACTGATCAATGGGATCCGAGACAAGC
AGAGTGGAAAGACAATCCTGGATTTTCTTAAGTCCGATGGATTTGCCAACCGGAACTTCATGCAGTTGAT
CCATGATGACTCTCTCACCTTTAAGGAGGACATCCAGAAAGCACAAGTTTCTGGCCAGGGGGACAGTCTT
CACGAGCACATCGCTAATCTTGCAGGTAGCCCAGCTATCAAAAAGGGAATACTGCAGACCGTTAAGGTCG
TGGATGAACTCGTCAAAGTAATGGGAAGGCATAAGCCCGAGAATATCGTTATCGAGATGGCCCGAGAGAA
CCAAACTACCCAGAAGGGACAGAAGAACAGTAGGGAAAGGATGAAGAGGATTGAAGAGGGTATAAAAGAA
CTGGGGTCCCAAATCCTTAAGGAACACCCAGTTGAAAACACCCAGCTTCAGAATGAGAAGCTCTACCTGT
ACTACCTGCAGAACGGCAGGGACATGTACGTGGATCAGGAACTGGACATCAATCGGCTCTCCGACTACGA
CGTGGCTGCTATCGTGCCCCAGTCTTTTCTCAAAGATGATTCTATTGATAATAAAGTGTTGACAAGATCC
GATAAAGCTAGAGGGAAGAGTGATAACGTCCCCTCAGAAGAAGTTGTCAAGAAAATGAAAAATTATTGGC
GGCAGCTGCTGAACGCCAAACTGATCACACAACGGAAGTTCGATAATCTGACTAAGGCTGAACGAGGTGG
CCTGTCTGAGTTGGATAAAGCCGGCTTCATCAAAAGGCAGCTTGTTGAGACACGCCAGATCACCAAGCAC
GTGGCCCAAATTCTCGATTCACGCATGAACACCAAGTACGATGAAAATGACAAACTGATTCGAGAGGTGA
AAGTTATTACTCTGAAGTCTAAGCTGGTCTCAGATTTCAGAAAGGACTTTCAGTTTTATAAGGTGAGAGA
GATCAACAATTACCACCATGCGCATGATGCCTACCTGAATGCAGTGGTAGGCACTGCACTTATCAAAAAA
TATCCCAAGCTTGAATCTGAATTTGTTTACGGAGACTATAAAGTGTACGATGTTAGGAAAATGATCGCAA
AGTCTGAGCAGGAAATAGGCAAGGCCACCGCTAAGTACTTCTTTTACAGCAATATTATGAATTTTTTCAA
GACCGAGATTACACTGGCCAATGGAGAGATTCGGAAGCGACCACTTATCGAAACAAACGGAGAAACAGGA
GAAATCGTGTGGGACAAGGGTAGGGATTTCGCGACAGTCCGGAAGGTCCTGTCCATGCCGCAGGTGAACA
TCGTTAAAAAGACCGAAGTACAGACCGGAGGCTTCTCCAAGGAAAGTATCCTCCCGAAAAGGAACAGCGA
CAAGCTGATCGCACGCAAAAAAGATTGGGACCCCAAGAAATACGGCGGATTCGATTCTCCTACAGTCGCT
TACAGTGTACTGGTTGTGGCCAAAGTGGAGAAAGGGAAGTCTAAAAAACTCAAAAGCGTCAAGGAACTGC
TGGGCATCACAATCATGGAGCGATCAAGCTTCGAAAAAAACCCCATCGACTTTCTCGAGGCGAAAGGATA
TAAAGAGGTCAAAAAAGACCTCATCATTAAGCTTCCCAAGTACTCTCTCTTTGAGCTTGAAAACGGCCGG
AAACGAATGCTCGCTAGTGCGGGCGAGCTGCAGAAAGGTAACGAGCTGGCACTGCCCTCTAAATACGTTA
ATTTCTTGTATCTGGCCAGCCACTATGAAAAGCTCAAAGGGTCTCCCGAAGATAATGAGCAGAAGCAGCT
GTTCGTGGAACAACACAAACACTACCTTGATGAGATCATCGAGCAAATAAGCGAATTCTCCAAAAGAGTG
ATCCTCGCCGACGCTAACCTCGATAAGGTGCTTTCTGCTTACAATAAGCACAGGGATAAGCCCATCAGGG
AGCAGGCAGAAAACATTATCCACTTGTTTACTCTGACCAACTTGGGCGCGCCTGCAGCCTTCAAGTACTT
CGACACCACCATAGACAGAAAGCGGTACACCTCTACAAAGGAGGTCCTGGACGCCACACTGATTCATCAG
TCAATTACGGGGCTCTATGAAACAAGAATCGACCTCTCTCAGCTCGGTGGAGACAGCAGGGCTGACCCCA
AGAAGAAGAGGAAGGTGGAGGCCAGCGGTTCCGGACGGGCTGACGCATTGGACGATTTTGATCTGGATAT
GCTGGGAAGTGACGCCCTCGATGATTTTGACCTTGACATGCTTGGTTCGGATGCCCTTGATGACTTTGAC
CTCGACATGCTCGGCAGTGACGCCCTTGATGATTTCGACCTGGACATGCTGATTAACTCTAGAAGTTCCG
GATCTCCGAAAAAGAAACGCAAAGTTGGTAGCCAGTACCTGCCCGACACCGACGACCGGCACCGGATCGA
GGAAAAGCGGAAGCGGACCTACGAGACATTCAAGAGCATCATGAAGAAGTCCCCCTTCAGCGGCCCCACC
GACCCTAGACCTCCACCTAGAAGAATCGCCGTGCCCAGCAGATCCAGCGCCAGCGTGCCAAAACCTGCCC
CCCAGCCTTACCCCTTCACCAGCAGCCTGAGCACCATCAACTACGACGAGTTCCCTACCATGGTGTTCCC
CAGCGGCCAGATCTCTCAGGCCTCTGCTCTGGCTCCAGCCCCTCCTCAGGTGCTGCCTCAGGCTCCTGCT
CCTGCACCAGCTCCAGCCATGGTGTCTGCACTGGCTCAGGCACCAGCACCCGTGCCTGTGCTGGCTCCTG
GACCTCCACAGGCTGTGGCTCCACCAGCCCCTAAACCTACACAGGCCGGCGAGGGCACACTGTCTGAAGC
TCTGCTGCAGCTGCAGTTCGACGACGAGGATCTGGGAGCCCTGCTGGGAAACAGCACCGATCCTGCCGTG
TTCACCGACCTGGCCAGCGTGGACAACAGCGAGTTCCAGCAGCTGCTGAACCAGGGCATCCCTGTGGCCC
CTCACACCACCGAGCCCATGCTGATGGAATACCCCGAGGCCATCACCCGGCTCGTGACAGGCGCTCAGAG
GCCTCCTGATCCAGCTCCTGCCCCTCTGGGAGCACCAGGCCTGCCTAATGGACTGCTGTCTGGCGACGAG
GACTTCAGCTCTATCGCCGATATGGATTTCTCAGCCTTGCTGGGCTCTGGCAGCGGCAGCCGGGATTCCA
GGGAAGGGATGTTTTTGCCGAAGCCTGAGGCCGGCTCCGCTATTAGTGACGTGTTTGAGGGCCGCGAGGT
GTGCCAGCCAAAACGAATCCGGCCATTTCATCCTCCAGGAAGTCCATGGGCCAACCGCCCACTCCCCGCC
AGCCTCGCACCAACACCAACCGGTCCAGTACATGAGCCAGTCGGGTCACTGACCCCGGCACCAGTCCCTC
AGCCACTGGATCCAGCGCCCGCAGTGACTCCCGAGGCCAGTCACCTGTTGGAGGATCCCGATGAAGAGAC
GAGCCAGGCTGTCAAAGCCCTTCGGGAGATGGCCGATACTGTGATTCCCCAGAAGGAAGAGGCTGCAATC
TGTGGCCAAATGGACCTTTCCCATCCGCCCCCAAGGGGCCATCTGGATGAGCTGACAACCACACTTGAGT
CCATGACCGAGGATCTGAACCTGGACTCACCCCTGACCCCGGAATTGAACGAGATTCTGGATACCTTCCT
GAACGACGAGTGCCTCTTGCATGCCATGCATATCAGCACAGGACTGTCCATCTTCGACACATCTCTGTTT
TGAccgactctagatcataatcagccataccacatttgtagaggttttacttgctttaaaaaacctccca
cacctccccctgaacctgaaacataaaatgaatgcaattgttgttgttaacttgtttattgcagcttata
atggttacaaataaagcaatagcatcacaaatttcacaaataaagcatttttttcactgcattctagttg
tggtttgcccaaactcatcaatgtatcttaGCGGCTCGAGGGTACCTCTAGAGATCCACTAGTGTCGACG
ATGTAGGTCACGGTCTCGAAGCCGCGGTGCGGGTGCCAGGGCGTGCCCTTGGGCTCCCCGGGCGCGTACT
CCACCTCACCCATCTGGTCCATCATGATGAACGGGTCGAGGTGGCGGTAGTTGATCCCGGCGAACGCGCG
GCGCACCGGGAAGCCCTCGCCCTCGAAACCGCTGGGCGCGGTGGTCACGGTGAGCACGGGACGTGCGACG
GCGTCGGCGGGTGCGGATACGCGGGGCAGCGTCAGCGGGTTCTCGACGGTCACGGCGGGCATGTCGACAC
TA
SEQ ID NO: 6-pMM7-9-3
CCACNCACGTTTCGTAGTTGCTCTTTCGCTGTCTCCCACCCGCTNTCCGCAACACATTCACCTTTTGTTC
GACGACCNTNGGAGCGACTGTCGTTAGTTCCGCGCGATTCGGTTCGCTCAAATGGTTCCGAGTGGTTCAT
TTCGTCTCAATAGAAATTAGTAATAAATATTTGTATGTACAATTTATTTGCTCCAATATATTTGTATATA
TTTCCCTCACAGCTATATTTATTCTAATTTAATATTATGACTTTTTAAGGTAATTTTTTGTGACCTGTTC
GGAGTGATTAGCGTTACAATTTGAACTGAAAGTGACATCCAGTGTTTGTTCCTTGTGTAGATGCATCTCA
AAAAAATGGTGGGCATAATAGTGTTGTTTATATATATCAAAAATAACAACTATAATAATAAGAATACATT
TAATTTAGAAAATGCTTGGATTTCACTGGAACTAGGCTAGCATAACTTCGTATAATGTATGCTATACGAA
GTTATGCTAGCGGATCCGGGAATTGGGAATTCACGTAAGTACTGTCTGCAGCGTAAGCTTCGTACGTAGC
actctaaaacgtaaagaaaccacagaacccatacgagagaaagcttgtaattcaattgctgcggtccttt
ggttcattgtgctttgtgaattaaagaattaacgatgttgtggtcggctaagtgaaaaaaaaaacagttc
ttgtcgtatttgtttatagaaagtggataattgccaacaggatagatagtggagctcaatcgctggggtt
ccccgataagaaaccgcccataatggaagctcttgtgtgtgcaaatacccttgtgcggcaaaacttcagg
aatttttcactagttatgcttagatctaaccattgattaacttcacaacaataaagaatgtttcataggc
tctaaatcgagattttgtgaggcttctaatgattgggcattcagcattttttcaagaattttgtaaccga
ctcaaaaaatctttagaatggttggttattcggatcgcatatacttagcttgtttgtcttatttttattt
ggatgagcgccaaaattttgctgcgtcagtctggaaaaaattgaatcaaatgtgtatagttttatagaag
ttgggaagcggaatttatttatttatttaaatatttataattaaaaaaatgaaaatagtcacgttgttta
actagtcagtattcgaaccaacaaatgtaaaatgtatactggtttgtgtctaagctaagcttgtcatatt
aacggagctgccagatgttaggaagtggggatgccatacattattctaaatttgcgcgcaattttagaag
cttatcgtcgtcagaattacaaaaacaaattgaatatgaaaatgggttattgctacttcattattattgt
cacgatatatgataatttatacaaaatgtgataaatcccaaattgttaaataatgctttggcttgcttta
tacaaaaccactagataattaaaatataggtggcctaaattgttgcatgttgttttataattaatcagca
atttgatttggttgtgatcgaccaaatcagtgtgtataattgtagttaaaatgtaaagttcgtaatggat
tattgaatcgcatttcaaatttctttaaatgcgcccgggtcaatgaccttttgaggtgaccataaattga
aacttatttgtgcgacggcaaccctgttctgggactcgacatgatatcgatacgttaacaacaaagagtc
tggacgccatcattcttcctctttctcctgaattcgcagacagcgtggcgtcaggcatttcaaacggcaa
aaagaacctggcgataaggaaagatttaaaaggcaaaaatcgagtgatttgtgtgatttaacttaagGCa
atcttacaaaATGGACAAGAAGTACTCCATTGGGCTCGCTATCGGCACAAACAGCGTCGGCTGGGCCGTC
ATTACGGACGAGTACAAGGTGCCGAGCAAAAAATTCAAAGTTCTGGGCAATACCGATCGCCACAGCATAA
AGAAGAACCTCATTGGCGCCCTCCTGTTCGACTCCGGGGAGACGGCCGAAGCCACGCGGCTCAAAAGAAC
AGCACGGCGCAGATATACCCGCAGAAAGAATCGGATCTGCTACCTGCAGGAGATCTTTAGTAATGAGATG
GCTAAGGTGGATGACTCTTTCTTCCATAGGCTGGAGGAGTCCTTTTTGGTGGAGGAGGATAAAAAGCACG
AGCGCCACCCAATCTTTGGCAATATCGTGGACGAGGTGGCGTACCATGAAAAGTACCCAACCATATATCA
TCTGAGGAAGAAGCTTGTAGACAGTACTGATAAGGCTGACTTGCGGTTGATCTATCTCGCGCTGGCGCAT
ATGATCAAATTTCGGGGACACTTCCTCATCGAGGGGGACCTGAACCCAGACAACAGCGATGTCGACAAAC
TCTTTATCCAACTGGTTCAGACTTACAATCAGCTTTTCGAAGAGAACCCGATCAACGCATCCGGAGTTGA
CGCCAAAGCAATCCTGAGCGCTAGGCTGTCCAAATCCCGGCGGCTCGAAAACCTCATCGCACAGCTCCCT
GGGGAGAAGAAGAACGGCCTGTTTGGTAATCTTATCGCCCTGTCACTCGGGCTGACCCCCAACTTTAAAT
CTAACTTCGACCTGGCCGAAGATACCAAGCTTCAACTGAGCAAAGACACCTACGATGATGATCTCGACAA
TCTGCTGGCCCAGATCGGCGACCAGTACGCAGACCTTTTTTTGGCGGCAAAGAACCTGTCAGACGCCATT
CTGCTGAGTGATATTCTGCGAGTGAACACGGAGATCACCAAAGCTCCGCTGAGCGCTAGTATGATCAAGC
TCTATGATGAGCACCACCAAGACTTGACTTTGCTGAAGGCCCTTGTCAGACAGCAACTGCCTGAGAAGTA
CAAGGAAATTTTCTTCGATCAGTCTAAAAATGGCTACGCCGGATACATTGACGGCGGAGCAAGCCAGGAG
GAATTTTACAAATTTATTAAGCCCATCTTGGAAAAAATGGACGGCACCGAGGAGCTGCTGGTAAAGCTTA
ACAGAGAAGATCTGTTGCGCAAACAGCGCACTTTCGACAATGGAATCATCCCCCACCAGATTCACCTGGG
CGAACTGCACGCTATCCTCAGGCGGCAAGAGGATTTCTACCCCTTTTTGAAAGATAACAGGGAAAAGATT
GAGAAAATCCTCACATTTCGGATACCCTACTATGTAGGCCCCCTCGCCCGGGGAAATTCCAGATTCGCGT
GGATGACTCGCAAATCAGAAGAAACCATCACTCCCTGGAACTTCGAGAAAGTCGTGGATAAGGGGGCCTC
TGCCCAGTCCTTCATCGAAAGGATGACTAACTTTGATAAAAATCTGCCTAACGAAAAGGTGCTTCCTAAA
CACTCTCTGCTGTACGAGTACTTCACAGTTTATAACGAGCTCACCAAGGTCAAATACGTCACAGAAGGGA
TGAGAAAGCCAGCATTCCTGTCTGGAGATCAGAAGAAAGCTATCGTGGACCTCCTCTTCAAGACGAACCG
GAAAGTTACCGTGAAACAGCTCAAAGAAGACTATTTCAAAAAGATTGAATGTTTCGACTCTGTTGAAATC
AGCGGAGTGGAGGATCGCTTCAACGCATCCCTGGGAACGTATCACGATCTCCTGAAAATCATTAAAGACA
AGGACTTCCTGGACAATGAGGAGAACGAGGACATTCTTGAGGACATTGTCCTCACCCTTACGTTGTTTGA
AGATAGGGAGATGATTGAAGAACGCTTGAAAACTTACGCTCATCTCTTCGACGACAAAGTCATGAAACAG
CTCAAGAGGCGCCGATATACAGGATGGGGGCGGCTGTCAAGAAAACTGATCAATGGGATCCGAGACAAGC
AGAGTGGAAAGACAATCCTGGATTTTCTTAAGTCCGATGGATTTGCCAACCGGAACTTCATTCAGTTGAT
CCATGATGACTCTCTCACCTTTAAGGAGGACATCCAGAAAGCACAAGTTTCTGGCCAGGGGGACAGTCTT
CACGAGCACATCGCTAATCTTGCAGGTAGCCCAGCTATCAAAAAGGGAATACTGCAGACCGTTAAGGTCG
TGGATGAACTCGTCAAAGTAATGGGAAGGCATAAGCCCGAGAATATCGTTATCGAGATGGCCCGAGAGAA
CCAAACTACCCAGAAGGGACAGAAGAACAGTAGGGAAAGGATGAAGAGGATTGAAGAGGGTATAAAAGAA
CTGGGGTCCCAAATCCTTAAGGAACACCCAGTTGAAAACACCCAGCTTCAGAATGAGAAGCTCTACCTGT
ACTACCTGCAGAACGGCAGGGACATGTACGTGGATCAGGAACTGGACATCAATCGGCTCTCCGACTACGA
CGTGGCTGCTATCGTGCCCCAGTCTTTTCTCAAAGATGATTCTATTGATAATAAAGTGTTGACAAGATCC
GATAAAgcTAGAGGGAAGAGTGATAACGTCCCCTCAGAAGAAGTTGTCAAGAAAATGAAAAATTATTGGC
GGCAGCTGCTGAACGCCAAACTGATCACACAACGGAAGTTCGATAATCTGACTAAGGCTGAACGAGGTGG
CCTGTCTGAGTTGGATAAAGCCGGCTTCATCAAAAGGCAGCTTGTTGAGACACGCCAGATCACCAAGCAC
GTGGCCCAAATTCTCGATTCACGCATGAACACCAAGTACGATGAAAATGACAAACTGATTCGAGAGGTGA
AAGTTATTACTCTGAAGTCTAAGCTGGTTTCAGATTTCAGAAAGGACTTTCAGTTTTATAAGGTGAGAGA
GATCAACAATTACCACCATGCGCATGATGCCTACCTGAATGCAGTGGTAGGCACTGCACTTATCAAAAAA
TATCCCAAGCTTGAATCTGAATTTGTTTACGGAGACTATAAAGTGTACGATGTTAGGAAAATGATCGCAA
AGTCTGAGCAGGAAATAGGCAAGGCCACCGCTAAGTACTTCTTTTACAGCAATATTATGAATTTTTTCAA
GACCGAGATTACACTGGCCAATGGAGAGATTCGGAAGCGACCACTTATCGAAACAAACGGAGAAACAGGA
GAAATCGTGTGGGACAAGGGTAGGGATTTCGCGACAGTCCGGAAGGTCCTGTCCATGCCGCAGGTGAACA
TCGTTAAAAAGACCGAAGTACAGACCGGAGGCTTCTCCAAGGAAAGTATCCTCCCGAAAAGGAACAGCGA
CAAGCTGATCGCACGCAAAAAAGATTGGGACCCCAAGAAATACGGCGGATTCGATTCTCCTACAGTCGCT
TACAGTGTACTGGTTGTGGCCAAAGTGGAGAAAGGGAAGTCTAAAAAACTCAAAAGCGTCAAGGAACTGC
TGGGCATCACAATCATGGAGCGATCAAGCTTCGAAAAAAACCCCATCGACTTTCTCGAGGCGAAAGGATA
TAAAGAGGTCAAAAAAGACCTCATCATTAAGCTTCCCAAGTACTCTCTCTTTGAGCTTGAAAACGGCCGG
AAACGAATGCTCGCTAGTGCGGGCGTGCTGCAGAAAGGTAACGAGCTGGCACTGCCCTCTAAATACGTTA
ATTTCTTGTATCTGGCCAGCCACTATGAAAAGCTCAAAGGTTCTCCCGAAGATAATGAGCAGAAGCAGCT
GTTCGTGGAACAACACAAACACTACCTTGATGAGATCATCGAGCAAATAAGCGAATTCTCCAAAAGAGTG
ATCCTCGCCGACGCTAACCTCGATAAGGTGCTTTCTGCTTACAATAAGCACAGGGATAAGCCCATCAGGG
AGCAGGCAGAAAACATTATCCACTTGTTTACTCTGACCAACTTGGGCGCGCCTGCAGCCTTCAAGTACTT
CGACACCACCATAGACAGAAAGCGGTACACCTCTACAAAGGAGGTCCTGGACGCCACACTGATTCATCAG
TCAATTACGGGGCTCTATGAAACAAGAATCGACCTCTCTCAGCTCGGTGGAGACAGCAGGGCTGACCCCA
AGAAGAAGAGGAAGGTGGAGGCCAGCGGTTCCGGACGGGCTGACGCATTGGACGATTTTGATCTGGATAT
GCTGGGAAGTGACGCCCTCGATGATTTTGACCTTGACATGCTTGGTTCGGATGCCCTTGATGACTTTGAC
CTCGACATGCTCGGCAGTGACGCCCTTGATGATTTCGACCTGGACATGCTGATTAACTCTAGAAGTTCCG
GATCTCCGAAAAAGAAACGCAAAGTTGGTAGCCAGTACCTGCCCGACACCGACGACCGGCACCGGATCGA
GGAAAAGCGGAAGCGGACCTACGAGACATTCAAGAGCATCATGAAGAAGTCCCCCTTCAGCGGCCCCACC
GACCCTAGACCTCCACCTAGAAGAATCGCCGTGCCCAGCAGATCCAGCGCCAGCGTGCCAAAACCTGCCC
CCCAGCCTTACCCCTTCACCAGCAGCCTGAGCACCATCAACTACGACGAGTTCCCTACCATGGTGTTCCC
CAGCGGCCAGATCTCTCAGGCCTCTGCTCTGGCTCCAGCCCCTCCTCAGGTGCTGCCTCAGGCTCCTGCT
CCTGCACCAGCTCCAGCCATGGTGTCTGCACTGGCTCAGGCACCAGCACCCGTGCCTGTGCTGGCTCCTG
GACCTCCACAGGCTGTGGCTCCACCAGCCCCTAAACCTACACAGGCCGGCGAGGGCACACTGTCTGAAGC
TCTGCTGCAGCTGCAGTTCGACGACGAGGATCTGGGAGCCCTGCTGGGAAACAGCACCGATCCTGCCGTG
TTCACCGACCTGGCCAGCGTGGACAACAGCGAGTTCCAGCAGCTGCTGAACCAGGGCATCCCTGTGGCCC
CTCACACCACCGAGCCCATGCTGATGGAATACCCCGAGGCCATCACCCGGCTCGTGACAGGCGCTCAGAG
GCCTCCTGATCCAGCTCCTGCCCCTCTGGGAGCACCAGGCCTGCCTAATGGACTGCTGTCTGGCGACGAG
GACTTCAGCTCTATCGCCGACATGGACTTCTCCGCACTGCTGGGTAGCGGATCGGGATCTCGGGATTCCA
GGGAAGGGATGTTTTTGCCGAAGCCTGAGGCCGGCTCCGCTATTAGTGACGTGTTTGAGGGCCGCGAGGT
GTGCCAGCCAAAACGAATCCGGCCATTTCATCCTCCAGGAAGTCCATGGGCCAACCGCCCACTCCCCGCC
AGCCTCGCACCAACACCAACCGGTCCAGTACATGAGCCAGTCGGGTCACTGACCCCGGCACCAGTCCCTC
AGCCACTGGATCCAGCGCCCGCAGTGACTCCCGAGGCCAGTCACCTGTTGGAGGATCCCGATGAAGAGAC
GAGCCAGGCTGTCAAAGCCCTTCGGGAGATGGCCGATACTGTGATTCCCCAGAAGGAAGAGGCTGCAATC
TGTGGCCAAATGGACCTTTCCCATCCGCCCCCAAGGGGCCATCTGGATGAGCTGACAACCACACTTGAGT
CCATGACCGAGGATCTGAACCTGGACTCACCCCTGACCCCGGAATTGAACGAGATTCTGGATACCTTCCT
GAACGACGAGTGCCTCTTGCATGCCATGCATATCAGCACAGGACTGTCCATCTTCGACACATCTCTGTTT
TGAccgactctagatcataatcagccataccacatttgtagaggttttacttgctttaaaaaacctccca
cacctccccctgaacctgaaacataaaatgaatgcaattgttgttgttaacttgtttattgcagcttata
atggttacaaataaagcaatagcatcacaaatttcacaaataaagcatttttttcactgcattctagttg
tggtttgcccaaactcatcaatgtatcttaGCGGCTCGAGGGTACCTCTAGAGATCCACTAGTGTCGACG
ATGTAGGTCACGGTCTCGAAGCCGCGGTGCGGGTGCCAGGGCGTGCCCTTGGGCTCCCCGGGCGCGTACT
CCACCTCACCCATCTGGTCCATCATGATGAACGGGTCGAGGTGGCGGTAGTTGATCCCGGCGAACGCGCG
GCGCACCGGGAAGCCCTCGCCCTCGAAACCGCTGGGCGCGGTGGTCACGGTGAGCACGGGACGTGCGACG
GCGTCGGCGGGTGCGGATACGCGGGGCAGCGTCAGCGGGTTCTCGACGGTCACGGCGGGCATGTCGACAC
TA

1. A biocontainment system comprising:
a polynucleotide encoding a coding region whose expression causes infertility or death;
a transcription regulatory region operably linked upstream of the coding region, and comprising a silent mutation; and
a polynucleotide that encodes a programmable transcription activator engineered to bind to the transcription regulatory region in the absence of the silent mutation, thereby expressing the coding region in the absence of the silent mutation, but does not initiate expression of the coding region when the transcription regulatory region comprises the silent mutation.

2. The biocontainment system of claim 1, wherein the programmable transcription activator comprises dCas9 fused to an activation domain.

3. The biocontainment system of claim 1, wherein the coding region encodes a cytoskeletal polypeptide, an ER-Golgi vesicle polypeptide, an mRNA processing polypeptide, an electron transport polypeptide, a nuclear trafficking polypeptide, a chromosome segregation polypeptide, a spindle pole duplication polypeptide, an oxidative stress polypeptide, or a polypeptide controlling development.

4. A multicellular organism comprising germ cells homozygous for the biocontainment system of claim 1.

5. A method of limiting hybridization of a genetically-modified organism with a genetically dissimilar variant, the method comprising:
providing an organism genetically modified to include the biocontainment system of claim 1, wherein a cross between the genetically-modified organism and the genetically dissimilar variant organism results in progeny that exhibit a phenotype that is distinct from the genetically-modified organism.

6. The method of claim 5, wherein the genetically dissimilar variant comprises a wild-type organism.

7. The method of claim 5, wherein the genetically dissimilar variant comprises a different genetic modification compared to the genetically-modified organism having the biocontainment system.

8. The method of claim 5, wherein the phenotype exhibited by the progeny comprises lethality.

9. An engineered genetic incompatibility (EGI) strain of a multicellular organism, the EGI strain comprising:
a haplosufficient lethal allele; and
a haploinsufficient resistance allele.

10. A method of suppressing a population of a wild-type organisms, the method comprising:
providing an engineered genetic incompatibility (EGI) strain of the wild-type organism, the EGI strain comprising:
a haplosufficient lethal allele; and
a haploinsufficient resistance allele;
so that wild-type×EGI crosses produce at least 50% lethality; and
mating members of the EGI strain of one sex with fertile adults of the opposite sex in the population of wild-type organisms.

11. The method of claim 10, further comprising:
mating members of the EGI strain of the one sex with fertile adults of the opposite sex in the wild-type population.

12. A method of replacing a population of wild-type organisms, the method comprising:
providing an engineered genetic incompatibility (EGI) strain of the wild-type organism, the EGI strain comprising:
a haplosufficient lethal allele; and
a haploinsufficient resistance allele;
so that wild-type×EGI crosses produce at least 50% lethality and EGI×EGI crosses produce at least 75% viability; and
mating the EGI strain with fertile adults in the population of wild-type organisms.

13. The multicellular organism of claim 4, wherein the programmable transcription activator comprises dCas9 fused to an activation domain.

14. The multicellular organism of claim 4, wherein the coding region encodes a cytoskeletal polypeptide, an ER-Golgi vesicle polypeptide, an mRNA processing polypeptide, an electron transport polypeptide, a nuclear trafficking polypeptide, a chromosome segregation polypeptide, a spindle pole duplication polypeptide, an oxidative stress polypeptide, or a polypeptide controlling development.

15. The method of claim 5, wherein the programmable transcription activator comprises dCas9 fused to an activation domain.

16. The method of claim 5, wherein the coding region encodes a cytoskeletal polypeptide, an ER-Golgi vesicle polypeptide, an mRNA processing polypeptide, an electron transport polypeptide, a nuclear trafficking polypeptide, a chromosome segregation polypeptide, a spindle pole duplication polypeptide, an oxidative stress polypeptide, or a polypeptide controlling development.


SEQUENCE LISTING (6 sequences)

SEQ ID NO: 1 - DNA, 6869 bp, Artificial
  Plasmid
  misc_feature (5)..(5): n is a, c, g, or t
  misc_feature (45)..(45): n is a, c, g, or t
  misc_feature (78)..(78): n is a, c, g, or t
  misc_feature (80)..(80): n is a, c, g, or t
ccacncacgtttcgtagttgctctttcgctgtctcccacccgctntccgcaacacattcaccttttgttcgacgaccntnggagcgactgtcgttagttccgcgcgattcggttcgctcaaatggttccgagtggttcatttcgtctcaatagaaattagtaataaatatttgtatgtacaatttatttgctccaatatatttgtatatatttccctcacagctatatttattctaatttaatattatgactttttaaggtaattttttgtgacctgttcggagtgattagcgttacaatttgaactgaaagtgacatccagtgtttgttccttgtgtagatgcatctcaaaaaaatggtgggcataatagtgttgtttatatatatcaaaaataacaactataataataagaatacatttaatttagaaaatgcttggatttcactggaactaggctagcataacttcgtataatgtatgctatacgaagttatgctagcggatccgggaattgggaattcacgtaagtactgtctgcagcgtaagcttcgtacgtagcggccgcaatcttacaaaatggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgataaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggctgctatcgtgccccagtcttttctcaaagatgattctattgataataaagtgttgacaagatccgataaagctagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttgaccgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgcccaaactcatcaatgtatcttagcggctcgagggtacctctagagatccactagtgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacacta

SEQ ID NO: 2 - DNA, 9370 bp, Artificial
  Plasmid
  misc_feature (5)..(5): n is a, c, g, or t
  misc_feature (45)..(45): n is a, c, g, or t
  misc_feature (78)..(78): n is a, c, g, or t
  misc_feature (80)..(80): n is a, c, g, or t
ccacncacgtttcgtagttgctctttcgctgtctcccacccgctntccgcaacacattcaccttttgttcgacgaccntnggagcgactgtcgttagttccgcgcgattcggttcgctcaaatggttccgagtggttcatttcgtctcaatagaaattagtaataaatatttgtatgtacaatttatttgctccaatatatttgtatatatttccctcacagctatatttattctaatttaatattatgactttttaaggtaattttttgtgacctgttcggagtgattagcgttacaatttgaactgaaagtgacatccagtgtttgttccttgtgtagatgcatctcaaaaaaatggtgggcataatagtgttgtttatatatatcaaaaataacaactataataataagaatacatttaatttagaaaatgcttggatttcactggaactaggctagcataacttcgtataatgtatgctatacgaagttatgctagcggatccgggaattgggaattcacgtaagtactgtctgcagcgtaagcttcgtacgtagcgtcaaatttggttgtgattacgagacggagaccgagacggcgacgacagttagccattcgccacgcgccaacgcaaatgaaacgctctatacatatttttgtatattttctgtttttttttgccgctgacaattatgatcaagtattagctggcgatagctgaaacgtctgtgtaatttcaatggaatggaatgggaagtgggcggcctattgatacactgctcgagtgattttaacttttatctgatcattcaaacgcataaattagtcttgagaacttcaattcatttgatactccagttaacatgctatttacatgctcatttaaatggtagtagtgatttataagcccacttccagatggaacttacctataccaacgtgttacttatcgttcttaagccaacttaatagcattctaaaatatatatgtatcttttggcggacttatcttcttgttgttctcgcattccaaaatctctatgtacatgcaaacttttattgtcataactcgggactttgcagactttgaggcctatttaatagagctataatcttacaacaaaaaaaaactaaaagagctttttaagcaataaaaatattctgaaaaattacaaattaacaaaaaattacccaatgaagcctgcaaatttgaaatctttaagatcctagatatgccaagatgcaccctaaagtccttaactcatctccttggctcgtttctaatcccccctctcgagggatcgagacgatcgcatcgggtcggtctttaagtttggatgatccataaactgttggtttctccgtcctcagcgtctagacttcattagccgtgtaatgttgcggaatttatgtggcaggcacattaaaataacaccgatacacactctcatggacgcgaacgtgtgtacaagtatagagatatcgggcctaggcgaaaaatgaaattaaaaaaaaaaaaaaaactggcaccgggaggggcttatttttcggtggtcggggatgcgggggactttgaccataaaatacatgctcccaaaaagctcgcacactgcaagagatgcggggcacttctgagtcccatattcatatgcacaaatgtgcattgctggcattatcagtagaatgcaatttcgggaaattttccatcgcatcacgagacaatgaacgtaagagagaaatggagcctcaaagagggagggagagagagagcttgagtgaacgagcgagcgacaatcgcgagataacggctgccttatcagcaatgccgaccgccccatcaccccacccaaaacgcccaaccaccacccaccgccgccgttccctttcctccatcgtcgagaatttcgagttcagagcagcgcgaccgaaatgaaaagaaacaatttaaattccaaatgtataaaataggtaaactatggcttttatttattaatattgacgggggcacaaaggcggtcacctcaatagtgaataacatgttttttataatgaatacttttcaaattgttattaatatgcaatgacgtcttaaatgtttcactgcagctgaactttattcttttcattaaaacagtcacccgttattaaaaataaatagattaagttttatattattaaatttgtaagtattgaaacaattccttttttattttatatgaattatcatttagttggggttaatatcccttaaagagagaaatttgtatgctttaagatttaaaatatctattgcatttatagctatagctataacttctcttatttcacgcagaaaatactcaaataaaacatatcgatttggcataccccactaattttttggccccaagtgtgtgagagtgtgtgaggcgaagcgcgccacaaacataaaaaagcggtgaagtgagcggttgtggaacgtgagtggatgctaagagcaagctctcacatacgcggacataggtcgcacacacacacgcacagaccgcctttttgcgccgccgaaacgaacacttttacgaaggcgacggcgaatcagtttcagttgtcagttcgcatccaactagaaagcagttaacgagtagtctgtgttttttcgcttgcggttaaaagccacgaggtcgttcatcgttcatcgttttccttttcaacttcaagcaaagcaaatataaaccaatgcaaaaaacgcagtgatcttttgaggcccaaatcgtttggggccgaacaccgttgattctaaaacgcaaatgtagaaacaaatcaagaaagtggaaaataaatatgtttcgctttcaaaacatgtgaatgtgccgaactcaaaactgaaacgtagaaggaacgcgttcgttttttacatacgacaatcgtataaaataagagaaaagctccaaaacgtattaaatagcgatgcttggatgatcttcgtagcagtcacgttgtacatacaaatacatacatatgtacctactatatggcacataaaatacgttacgcacactagtggcgaataaaaagcgaattggagcaatcttacaaaatggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgataaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggctgctatcgtgccccagtcttttctcaaagatgattctattgataataaagtgttgacaagatccgataaagctagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttgaccgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgcccaaactcatcaatgtatcttagcggctcgagggtacctctagagatccactagtgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacacta

SEQ ID NO: 3 - DNA, 7696 bp, Artificial
  Plasmid
  misc_feature (5)..(5): n is a, c, g, or t
  misc_feature (45)..(45): n is a, c, g, or t
  misc_feature (78)..(78): n is a, c, g, or t
  misc_feature (80)..(80): n is a, c, g, or t
ccacncacgtttcgtagttgctctttcgctgtctcccacccgctntccgcaacacattcaccttttgttcgacgaccntnggagcgactgtcgttagttccgcgcgattcggttcgctcaaatggttccgagtggttcatttcgtctcaatagaaattagtaataaatatttgtatgtacaatttatttgctccaatatatttgtatatatttccctcacagctatatttattctaatttaatattatgactttttaaggtaattttttgtgacctgttcggagtgattagcgttacaatttgaactgaaagtgacatccagtgtttgttccttgtgtagatgcatctcaaaaaaatggtgggcataatagtgttgtttatatatatcaaaaataacaactataataataagaatacatttaatttagaaaatgcttggatttcactggaactaggctagcataacttcgtataatgtatgctatacgaagttatgctagcggatccgggaattgggaattcacgtaagtactgtctgcagcgtaagcttcgtacgtagcgaccgtctcaaagtactgcctttctgcgttggaaaacatcgcctttttcgtccaaaaggagtccccaggttcgatccgcatggcgttgtgcgtgcgtgcctttcttttcaaatgattacggctattaacttgggggcgttaagttggaaacacgtaaattgcagactgcgattagagtgaccatgagtaggagttcaaaatctcctgacatcattttcttaaaacctgctttgttttttacatttctatttaatataactcctatttgaataaaaaaacaaaacaagtttagatgttaagatattaactacatcctttgctccaaagggagaggggaagttatggagttaattaatttgctgttggaaatcaatatggagtcagaaatataatgatttactaaaccttattgaatcggtaacgatgcgaatttatattaaaatagcttttatgaaacattcaacaaaaatattattaatgttggcccactttagcaaccggttaggtctaccggttgggcaagcaaagattcacgccctggttcgagtcccaactagtcctgcaaaataccgcagcaagttttagagagaccaagtgccattacctctcccacttcagttatcggttatgcggcgtttaagtcgacagcttgccgtctctagctccggtgcctatataaagcagcccgctttccacatttcatattcgttttacgtttgtcaagcctcatagccggcagttcgaacgtatacgctctctgagtcagacctcgaaatcgtagctctacacaattctgtgaattttccttgtcgcgtgtgaaacacttccaatgcaatcttacaaaatggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgataaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggctgctatcgtgccccagtcttttctcaaagatgattctattgataataaagtgttgacaagatccgataaagctagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttgaccgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgcccaaactcatcaatgtatcttagcggctcgagggtacctctagagatccactagtgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacacta

SEQ ID NO: 4 - DNA, 9411 bp, Artificial
  Plasmid
  misc_feature (5)..(5): n is a, c, g, or t
  misc_feature (45)..(45): n is a, c, g, or t
  misc_feature (78)..(78): n is a, c, g, or t
  misc_feature (80)..(80): n is a, c, g, or t
ccacncacgtttcgtagttgctctttcgctgtctcccacccgctntccgcaacacattcaccttttgttcgacgaccntnggagcgactgtcgttagttccgcgcgattcggttcgctcaaatggttccgagtggttcatttcgtctcaatagaaattagtaataaatatttgtatgtacaatttatttgctccaatatatttgtatatatttccctcacagctatatttattctaatttaatattatgactttttaaggtaattttttgtgacctgttcggagtgattagcgttacaatttgaactgaaagtgacatccagtgtttgttccttgtgtagatgcatctcaaaaaaatggtgggcataatagtgttgtttatatatatcaaaaataacaactataataataagaatacatttaatttagaaaatgcttggatttcactggaactaggctagcataacttcgtataatgtatgctatacgaagttatgctagcggatccgggaattgggaattcacgtaagtactgtctgcagcgtaagcttcgtacgtagccagctccttgttggttgaccaaatcgtagaccttcaataaatttccaaggacacgcaccactcgtttggaaaatatttggttctcttcaaggttgaaactctcgggggttttggacgtcgatcggttttggtttgtttagaattgttgtggcgttgttttacgaatagatattactttgatggattagccatatcgcattgaaggtcgcctcttggttagcctcgaatttgttacgacctgttttgtgttggctaaccaaaatacaaactccgcacatacagtgcgagcaacaattatgcagaaaaaacgcatgaatagcttatggaatttacactttgacggatgaagaaatatacttcctttttccatattcgatattatcgtagagatagaataagatatattgttctaaattccttttatgtcctactatttctttgatttgattaaaaatgtgtcttcccaagaagaacttaattgcctcagataattgcttatcgtaaagaaattaccaccactctcgctatctgccattagttaatgaatagacccaccagactttagtagctctgccgatttgggttatttttacaacctcggtgggcgagcgggatggaagacgaggagaggtgatcactacctacagcagcgagaggtttggattttatgatatatttacattccagccttctgttcttactcactcgccgtgaatgtctgggagtgcgtgtgtgtctcatcatttggattttccgcggcaataaaattaataagaacgcgctaatttttcaggccccggggcctaagcaaataaacatacactatttcctgcaactcctccacccttttcccctaactcttttccagcgcccagactgtgctaatatttgccaagggatattattgggcctaaaccgaaaacggaactctttccgttcgccattttgttggccagcaaagcgcttttcctgttgttgtttaccgatgaattgaaaaataaatgaatatatttatttggaacatttatgttttgtcctacactataattaatttaaaatcactatcagttctggcaggctctaaacagcgaattaatgtttaattcattgaaaatggctgaaaaaaaagtgttctataggtggggaagatagcccctaaaggtggggtgggataccagctcttcttgggctgcacaaactgtccaattagtggaagcggccaagcaatggatgaggaaaaggtaagacataaactcggttcggaatgccaaagtgtgtggtaacaatcccctgagagtgagggagctggctgcatccaagtgcagtatataagactactccgaaatttactccgaaaagcagcagaaaacttgttctgacacggcaaatgtatggaaaggtttaaggaaacaggcatattaaagaaacttcttgttaattgtttctaaatatttatatttatagagtagctaaatttagttgctatcgatttaagaatactttcatagccaaaagctagaagttaaaagtagtaataccactttttcacccataagctaaagataaaacccaaattcaacagtcgaaaataatagttcaaagcctttattagccgaacagtaagcgtaacaaaatcaccataaaaaaccaatcccataaatatcttacagaaataggcgaaaatattgcgacaaatatgtataattaaatgtagtcaaagctatgacgaaattcatgaggttgcgcaaataatcgggcaatacaatcgattacaccgaaaatgcaccgagtttttccatttccgccatttcttattgggccatgctggctatataccgcacacacacacacgcacgcacacttcaaagcgcaacacacaagaaacgtttacgaagagacagggagaacgaacgatcaccgcgccatatagcggtgctcttctggcgcacgcagctgcaatgcaggagtcagggtatagctccaccccactcgcacacacacaccatcgggcggtcgtgtatgcgatccgaagacgaagaccgacgatgcgatcggatcggggatctcgggtcgctgctgacaaacgcagagtcggacgaaagaacgcaccgtgtgtttcagttaagcgttggcactgaaccgggcaacaatcttcactcctccgctcgaaacgccgcgatcgaaccgatctataactagccatctataactagagcgagccgagtgtattctatcgaaacagccaaatttacgatacaatatatatttgtatatgcgtggaaaacttacaagttcttgttgtgtcccatgattgccgtgtgatccagcggaattaatcgcacaaatatgagcagcaatatcggcatacgcatgctaatgatgattatgcctcatttatagtgcgctaattgaacgcgaaattgctcgatacattcaatataaccaaaccattcgcaaacaaacaacaactcgaagggaagtatctatcataccccgtgtgtcagtgtgagagtgtgtgtgccgtcgaacagataaacccgatcagcgcaatcttacaaaatggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgataaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggctgctatcgtgccccagtcttttctcaaagatgattctattgataataaagtgttgacaagatccgataaagctagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttgaccgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgcccaaactcatcaatgtatcttagcggctcgagggtacctctagagatccactagtgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacacta

SEQ ID NO: 5 - DNA, 8192 bp, Artificial
  Plasmid
  misc_feature (5)..(5): n is a, c, g, or t
  misc_feature (45)..(45): n is a, c, g, or t
  misc_feature (78)..(78): n is a, c, g, or t
  misc_feature (80)..(80): n is a, c, g, or t
ccacncacgtttcgtagttgctctttcgctgtctcccacccgctntccgcaacacattcaccttttgttcgacgaccntnggagcgactgtcgttagttccgcgcgattcggttcgctcaaatggttccgagtggttcatttcgtctcaatagaaattagtaataaatatttgtatgtacaatttatttgctccaatatatttgtatatatttccctcacagctatatttattctaatttaatattatgactttttaaggtaattttttgtgacctgttcggagtgattagcgttacaatttgaactgaaagtgacatccagtgtttgttccttgtgtagatgcatctcaaaaaaatggtgggcataatagtgttgtttatatatatcaaaaataacaactataataataagaatacatttaatttagaaaatgcttggatttcactggaactaggctagcataacttcgtataatgtatgctatacgaagttatgctagcggatccgggaattgggaattcacgtaagtactgtctgcagcgtaagcttcgtacgtagcactctaaaacgtaaagaaaccacagaacccatacgagagaaagcttgtaattcaattgctgcggtcctttggttcattgtgctttgtgaattaaagaattaacgatgttgtggtcggctaagtgaaaaaaaaaacagttcttgtcgtatttgtttatagaaagtggataattgccaacaggatagatagtggagctcaatcgctggggttccccgataagaaaccgcccataatggaagctcttgtgtgtgcaaatacccttgtgcggcaaaacttcaggaatttttcactagttatgcttagatctaaccattgattaacttcacaacaataaagaatgtttcataggctctaaatcgagattttgtgaggcttctaatgattgggcattcagcattttttcaagaattttgtaaccgactcaaaaaatctttagaatggttggttattcggatcgcatatacttagcttgtttgtcttatttttatttggatgagcgccaaaattttgctgcgtcagtctggaaaaaattgaatcaaatgtgtatagttttatagaagttgggaagcggaatttatttatttatttaaatatttataattaaaaaaatgaaaatagtcacgttgtttaactagtcagtattcgaaccaacaaatgtaaaatgtatactggtttgtgtctaagctaagcttgtcatattaacggagctgccagatgttaggaagtggggatgccatacattattctaaatttgcgcgcaattttagaagcttatcgtcgtcagaattacaaaaacaaattgaatatgaaaatgggttattgctacttcattattattgtcacgatatatgataatttatacaaaatgtgataaatcccaaattgttaaataatgctttggcttgctttatacaaaaccactagataattaaaatataggtggcctaaattgttgcatgttgttttataattaatcagcaatttgatttggttgtgatcgaccaaatcagtgtgtataattgtagttaaaatgtaaagttcgtaatggattattgaatcgcatttcaaatttctttaaatgcgcccgggtcaatgaccttttgaggtgaccataaattgaaacttatttgtgcgacggcaaccctgttctgggactcgacatgatatcgatacgttaacaacaaagagtctggacgccatcattcttcctctttctcctgaattcgcagacagcgtggcgtcaggcatttcaaacggcaaaaagaacctggcgataaggaaagatttaaaaggcaaaaatcgagtgatttgtgtgatttaacttaaggcaatcttacaaaatggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgataaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggctgctatcgtgccccagtcttttctcaaagatgattctattgataataaagtgttgacaagatccgataaagctagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttgaccgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgcccaaactcatcaatgtatcttagcggctcgagggtacctctagagatccactagtgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacacta

SEQ ID NO: 6 - DNA, 8192 bp, Artificial
  Plasmid
  misc_feature (5)..(5): n is a, c, g, or t
  misc_feature (45)..(45): n is a, c, g, or t
  misc_feature (78)..(78): n is a, c, g, or t
  misc_feature (80)..(80): n is a, c, g, or t
ccacncacgtttcgtagttgctctttcgctgtctcccacccgctntccgcaacacattcaccttttgttcgacgaccntnggagcgactgtcgttagttccgcgcgattcggttcgctcaaatggttccgagtggttcatttcgtctcaatagaaattagtaataaatatttgtatgtacaatttatttgctccaatatatttgtatatatttccctcacagctatatttattctaatttaatattatgactttttaaggtaattttttgtgacctgttcggagtgattagcgttacaatttgaactgaaagtgacatccagtgtttgttccttgtgtagatgcatctcaaaaaaatggtgggcataatagtgttgtttatatatatcaaaaataacaactataataataagaatacatttaatttagaaaatgcttggatttcactggaactaggctagcataacttcgtataatgtatgctatacgaagttatgctagcggatccgggaattgggaattcacgtaagtactgtctgcagcgtaagcttcgtacgtagcactctaaaacgtaaagaaaccacagaacccatacgagagaaagcttgtaattcaattgctgcggtcctttggttcattgtgctttgtgaattaaagaattaacgatgttgtggtcggctaagtgaaaaaaaaaacagttcttgtcgtatttgtttatagaaagtggataattgccaacaggatagatagtggagctcaatcgctggggttccccgataagaaaccgcccataatggaagctcttgtgtgtgcaaatacccttgtgcggcaaaacttcaggaatttttcactagttatgcttagatctaaccattgattaacttcacaacaataaagaatgtttcataggctctaaatcgagattttgtgaggcttctaatgattgggcattcagcattttttcaagaattttgtaaccgactcaaaaaatctttagaatggttggttattcggatcgcatatacttagcttgtttgtcttatttttatttggatgagcgccaaaattttgctgcgtcagtctggaaaaaattgaatcaaatgtgtatagttttatagaagttgggaagcggaatttatttatttatttaaatatttataattaaaaaaatgaaaatagtcacgttgtttaactagtcagtattcgaaccaacaaatgtaaaatgtatactggtttgtgtctaagctaagcttgtcatattaacggagctgccagatgttaggaagtggggatgccatacattattctaaatttgcgcgcaattttagaagcttatcgtcgtcagaattacaaaaacaaattgaatatgaaaatgggttattgctacttcattattattgtcacgatatatgataatttatacaaaatgtgataaatcccaaattgttaaataatgctttggcttgctttatacaaaaccactagataattaaaatataggtggcctaaattgttgcatgttgttttataattaatcagcaatttgatttggttgtgatcgaccaaatcagtgtgtataattgtagttaaaatgtaaagttcgtaatggattattgaatcgcatttcaaatttctttaaatgcgcccgggtcaatgaccttttgaggtgaccataaattgaaacttatttgtgcgacggcaaccctgttctgggactcgacatgatatcgatacgttaacaacaaagagtctggacgccatcattcttcctctttctcctgaattcgcagacagcgtggcgtcaggcatttcaaacggcaaaaagaacctggcgataaggaaagatttaaaaggcaaaaatcgagtgatttgtgtgatttaacttaaggcaatcttacaaaatggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagataccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagctctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaatcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagaaaccatcactccctggaacttcgagaaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagatcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcattcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggctgctatcgtgccccagtcttttctcaaagatgattctattgataataaagtgttgacaagatccgataaagctagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtttcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgtgctgcagaaaggtaacgagctggcactgccctctaaatacgttaatttcttgtatctggccagccactatgaaaagctcaaaggttctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgacatggacttctccgcactgctgggtagcggatcgggatctcgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttgaccgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgcccaaactcatcaatgtatcttagcggctcgagggtacctctagagatccactagtgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacacta